(19) United States
(12) Patent Application Publication
(10) Pub. No.: US 2021/0179734 A1
(43) Pub. Date: Jun. 17, 2021

(54) TRIVALENT TRISPECIFIC ANTIBODY CONSTRUCTS

(71) Applicant: INVENRA INC., Madison, WI (US)

(72) Inventors: Lucas BAILEY, Suzhou (CN); Bryan GLASER, Fitchburg, WI (US); Qufei LI, Suzhou (CN); Roland GREEN, Madison, WI (US); Dileep Kumar PULUKKUNAT, Middleton, WI (US); Malgorzata Agnieszka NOCULA-LUGOWSKA, Brighton, MA (US); Daniel Justin GERHARDT, Madison, WI (US)

(21) Appl. No.: 17/048,482

(22) PCT Filed: Apr. 17, 2019
(86) PCT No.: PCT/US2019/027816

§ 371 Date: Oct. 16, 2020

Related U.S. Application Data
(60) Provisional application No. 62/659,047, filed on Apr. 17, 2018.

(51) Int. Cl. C07K 16/46

(52) U.S. Cl. CPC C07K 16/468; C07K 2317/31; C07K 2317/526; C07K 2317/35; C07K 2317/524; C07K 2317/64; C07K 2317/71; C07K 2317/522

(57) ABSTRACT

Trispecific trivalent antibody constructs, pharmaceutical compositions comprising the constructs, and methods of use thereof are presented.

### 1. CROSS-REFERENCE TO RELATED APPLICATIONS

This application claims the benefit of and priority to U.S. Provisional Application No. 62/659,047, filed Apr. 17, 2018. The content of the above referenced application is incorporated by reference in its entirety.

### 2. SEQUENCE LISTING

The instant application contains a Sequence Listing which has been submitted via EFS-Web and is hereby incorporated herein by reference in its entirety. Said ASCII copy, created on Month XX, 2019, is named XXXXXUS_sequencelisting.txt, and is X,XXX,XXX bytes in size.

### 3. BACKGROUND

Antibodies are an invaluable tool in the medical field. In particular, the importance of monoclonal antibodies, including their roles in scientific research and medical diagnostics, have been widely recognized for several decades. However, the full potential of antibodies, especially their successful use as therapeutic agents, has only more recently been demonstrated, as demonstrated by the successful therapies adalimumab (Humira), rituximab (Rituxan), infliximab (Remicade), bevacizumab (Avastin), trastuzumab (Herceptin), pembrolizumab (Keytruda), and ipilimumab (Yervoy). Following these clinical successes, interest in antibody therapies will likely only continue to increase. Therefore, a need for efficient generation and manufacturing of antibodies exists in the field, both in the research drug development and downstream clinical settings.

An area of active research in the antibody therapeutic field is the design and use of multispecific antibodies, i.e. a single antibody engineered to recognize multiple targets. These antibodies offer the promise of greater therapeutic control. For example, a need exists to improve target specificity in order to reduce the off-target effects associated with many antibody therapies, particularly in the case of antibody based immunotherapies. In addition, multispecific antibodies offer new therapeutic strategies, such as synergistic targeting of multiple cell receptors, especially in an immunotherapy context. One such immunotherapy is the use of bispecific antibodies to recruit T cells to target and kill specific tumor cell populations through bispecific engagement of a T cell marker and a tumor cell marker. For example, the targeting of B cell lymphoma using CD3×CD19 bispecific antibodies is described in U.S. Pub. No. 2006/0193852.

Despite the promise of multispecific antibodies, their production and use has been plagued by numerous constraints that have limited their practical implementation. In general, all multispecific antibody platforms must solve the problem of ensuring high fidelity pairing between cognate heavy and light chain pairs. However, a multitude of issues exist across the various platforms. For example, antibody chain engineering can result in poor stability of assembled antibodies, poor expression and folding of the antibody chains, and/or generation of immunogenic peptides. Other approaches suffer from impractical manufacturing processes, such as complicated in vitro assembly reactions or purification methods. In addition, several platforms suffer from the inability to easily and efficiently plug in different antibody binding domains. These various problems associated with multispecific antibody manufacturing limit the applicability of many platforms, especially their use in high-throughput screens necessary for many therapeutic drug pipelines, such as in screening for improved antigen binding specificity or affinity.

There is, therefore, a need for an antibody platform capable of high-level expression and efficient purification. In particular, there is a need for a multispecific antibody platform that improves the manufacturing capabilities of multispecific antibodies with direct applicability in both research and therapeutic settings. There is also a need for improved multispecific antibodies that specifically bind to distinct cell populations, including tumor cell populations, with improvements including increased affinity or avidity, reduced off-target binding, and/or reduced unintended immune activation.

### 4. SUMMARY

Disclosed herein is a trivalent trispecific binding molecule comprising: a first, a second, a third, a fourth, and a fifth polypeptide chain, wherein: (a) the first polypeptide chain comprises a domain A, a domain B, a domain D, a domain E, a domain N and a domain O, wherein the domains are arranged, from N-terminus to C-terminus, in an N-O-A-B-D-E orientation, and domain A has a variable region domain amino acid sequence, domain B has a constant region domain amino acid sequence, domain D has a CH2 amino acid sequence, domain E has a constant region domain amino acid sequence, domain N has a variable region domain amino acid sequence, and domain O has a constant region domain amino acid sequence; (b) the second polypeptide chain comprises a domain F and a domain G, wherein the domains are arranged, from N-terminus to C-terminus, in a F-G orientation, and wherein domain F has a variable region domain amino acid sequence and domain G has a constant region domain amino acid sequence amino acid sequence; (c) the third polypeptide chain comprises a domain H, a domain I, a domain J, and a domain K, wherein the domains are arranged, from N-terminus to C-terminus, in a H-I-J-K orientation, and wherein domain H has a variable region domain amino acid sequence, domain I has a constant region domain amino acid sequence, domain J has a CH2 amino acid sequence, and K has a constant region domain amino acid sequence; (d) the fourth polypeptide chain comprises a domain L and a domain M, wherein the domains are arranged, from N-terminus to C-terminus, in a L-M orientation, and wherein domain L has a variable region domain amino acid sequence and domain M has a constant region domain amino acid sequence; (e) the fifth polypeptide chain comprises a domain P and a domain Q, wherein the domains are arranged, from N-terminus to C-terminus, in a P-Q orientation, and wherein domain P has a variable region domain amino acid sequence and domain Q has a constant region domain amino acid sequence, (f) the first and the second polypeptides are associated through an interaction between the A and the F domains and an interaction between the B and the G domains; (g) the third and the fourth polypeptides are associated through an interaction between the H and the L domains and an interaction between the I and the M domains; (h) the first and the fifth polypeptides are associated through an interaction between the N and the P domains and an interaction between the O and the Q domains to form the binding molecule; (i) the first and the third polypeptides are associated through an interaction between the D and the J domains and an interaction between the E and the K domains to form the binding molecule; (j) the amino acid sequences of domain N, domain A, and domain H are different, (k) the second and the fifth polypeptide chains are identical and the fourth polypeptide chain is different, or the fourth and the fifth polypeptide chains are identical and the second polypeptide chain is different, and (l) the interaction between the A domain and the F domain form a first antigen binding site specific for a first antigen, the interaction between the H domain and the L domain form a second antigen binding site specific for a second antigen, and the interaction between the N domain and the P domain form a third antigen binding site specific for a third antigen.

In certain aspects, the second and the fifth polypeptide chains are identical and the fourth polypeptide chain is different from the second and the fifth polypeptide chains, the amino acid sequences of domain O and domain B are identical, and the amino acid sequences of domain I is different from domains O and B.

In certain aspects, the fourth and the fifth polypeptide chains are identical and the second polypeptide chain is different from the second and the fifth polypeptide chains, the amino acid sequences of domain O and domain I are identical, and the amino acid sequences of domain B is different from domains O and I.

Also disclosed herein is a trivalent trispecific binding molecule comprising: a first, a second, a third, a fourth, and a sixth polypeptide chain, wherein: (a) the first polypeptide chain comprises a domain A, a domain B, a domain D, and a domain E, wherein the domains are arranged, from N-terminus to C-terminus, in an A-B-D-E orientation, and domain A has a variable region domain amino acid sequence, domain B has a constant region domain amino acid sequence, domain D has a CH2 amino acid sequence, and domain E has a constant region domain amino acid sequence; (b) the second polypeptide chain comprises a domain F and a domain G, wherein the domains are arranged, from N-terminus to C-terminus, in a F-G orientation, and wherein domain F has a variable region domain amino acid sequence and domain G has a constant region domain amino acid sequence amino acid sequence; (c) the third polypeptide chain comprises a domain H, a domain I, a domain J, a domain K, a domain R, and a domain S wherein the domains are arranged, from N-terminus to C-terminus, in a R-S-H-I-J-K orientation, and wherein domain H has a variable region domain amino acid sequence, domain I has a constant region domain amino acid sequence, domain J has a CH2 amino acid sequence, domain K has a constant region domain amino acid sequence, domain R has a variable region domain amino acid sequence, and domain S has a constant region domain amino acid sequence; (d) the fourth polypeptide chain comprises a domain L and a domain M, wherein the domains are arranged, from N-terminus to C-terminus, in a L-M orientation, and wherein domain L has a variable region domain amino acid sequence and domain M has a constant region domain amino acid sequence; (e) the sixth polypeptide chain comprises a domain T and a domain U, wherein the domains are arranged, from N-terminus to C-terminus, in a T-U orientation, and wherein domain T has a variable region domain amino acid sequence and domain U has a constant region domain amino acid sequence, (f) the first and the second polypeptides are associated through an interaction between the A and the F domains and an interaction between the B and the G domains; (g) the third and the fourth polypeptides are associated through an interaction between the H and the L domains and an interaction between the I and the M domains; (h) the first and the sixth polypeptides are associated through an interaction between the R and the T domains and an interaction between the S and the U domains to form the binding molecule; (i) the first and the third polypeptides are associated through an interaction between the D and the J domains and an interaction between the E and the K domains to form the binding molecule; (j) the amino acid sequences of domain R, domain A, and domain H are different, (k) the second and the sixth polypeptide chains are identical and the fourth polypeptide chain is different, or the fourth and the sixth polypeptide chains are identical and the second polypeptide chain is different, (1) the interaction between the A domain and the F domain form a first antigen binding site specific for a first antigen, the interaction between the H domain and the L domain form a second antigen binding site specific for a second antigen, and the interaction between the R domain R and the T domain form a third antigen binding site specific for a third antigen.

In certain aspects, the fourth and the sixth polypeptide chains are identical and the fourth polypeptide chain is different from the second and the sixth polypeptide chains, the amino acid sequences of domain S and domain I are identical, and the amino acid sequences of domain B is different from domains S and I.

In certain aspects, the second and the sixth polypeptide chains are identical and the fourth polypeptide chain is different from the second and the sixth polypeptide chains, the amino acid sequences of domain S and domain B are identical, and the amino acid sequences of domain I is different from domains S and B.

Also disclosed herein is a purified binding molecule, the purified binding molecule comprising any of the binding molecules described herein. In certain aspects, the binding molecule is purified by a purification method comprising a CH1 affinity purification step. In certain aspects, the purification method is a single-step purification method.

Also disclosed herein is a pharmaceutical composition comprising any of the binding molecules described herein and a pharmaceutically acceptable diluent.

Also disclosed herein is a method for treating a subject with cancer, the method comprising administering a therapeutically effective amount of any of the pharmaceutical composition described herein.

### 5. BRIEF DESCRIPTION OF THE DRAWINGS

FIG. 1 shows an alignment of the CH3-CH3 IgG1 dimer pair with CH1-C_{L}. The quaternary structures align with an RMSD of ˜1.6 Å².

FIG. 2 presents schematic architectures, with respective naming conventions, for various binding molecules (also called antibody constructs) described herein.

FIG. 3 presents a higher resolution schematic of polypeptide chains and their domains, with respective naming conventions, for the bivalent 1×1 antibody constructs described herein.

FIG. 4 shows the architecture of an exemplary bivalent, monospecific, construct.

FIG. 5 shows data from a biolayer interferometry (BLI) experiment, described in Example 1, in which a bivalent monospecific binding molecule having the architecture illustrated in FIG. 4 [polypeptide 1: VL-CH3(Knob)-CH2-CH3/polypeptide 2: VH-CH3(Hole)] was assayed. The antigen binding site was specific for TNFα. The BLI response from binding molecule immobilization and TNFα binding to the immobilized construct demonstrates robust, specific, bivalent binding to the antigen. The data are consistent with a molecule having a high percentage of intended pairing of polypeptide 1 and polypeptide 2.

FIG. 6 illustrates features of an exemplary bivalent 1×1 bispecific binding molecule, “BC1”.

FIG. 7A shows size exclusion chromatography (SEC) analysis of “BC1”, demonstrating that a single-step CH1 affinity purification step (CaptureSelect™ CH1 affinity resin) yields a single, monodisperse peak via gel filtration in which >98% is unaggregated bivalent protein. FIG. 7B shows comparative literature data of SEC analysis of a CrossMab bivalent antibody construct [data from Schaefer et al. (Proc Natl Acad Sci USA. 2011 Jul. 5; 108(27):11187-92)].

FIG. 8A is a cation exchange chromatography elution profile of “BC1” following one-step purification using the CaptureSelect™ CH1 affinity resin, showing a single tight peak. FIG. 8B is a cation exchange chromatography elution profile of “BC1” following purification using standard Protein A purification.

FIG. 9 shows nonreducing SDS-PAGE gels of “BC1” at various stages of purification.

FIGS. 10A and 10B compare SDS-PAGE gels of “BC1” after single-step CH1-affinity purification under both non-reducing and reducing conditions (FIG. 10A) with SDS-PAGE gels of a CrossMab bispecific antibody under non-reducing and reducing conditions as published in the referenced literature (FIG. 10B).

FIGS. 11A and 11B show mass spec analysis of “BC1”, demonstrating two distinct heavy chains (FIG. 11A) and two distinct light chains (FIG. 11B) under reducing conditions.

FIG. 12 presents a mass spectrometry analysis of purified “BC1” under non-reducing conditions, confirming the absence of incomplete pairing after purification.

FIG. 13 presents accelerated stability testing data demonstrating stability of “BC1” over 8 weeks at 40° C., compared to two IgG control antibodies.

FIG. 14 illustrates features of an exemplary bivalent 1×1 bispecific binding molecule, “BC6”, further described in Example 3.

FIG. 15A presents size exclusion chromatography (SEC) analysis of “BC6” following one-step purification using the CaptureSelect™ CH1 affinity resin, demonstrating that the single step CH1 affinity purification yields a single monodisperse peak and the absence of non-covalent aggregates. FIG. 15B shows a SDS-PAGE gel of “BC6” under non-reducing conditions.

FIG. 16 illustrates features of an exemplary bivalent bispecific binding molecule, “BC28”, further described in Example 4.

FIG. 17 shows SDS-PAGE analysis under non-reducing conditions following single-step CH1 affinity purification of “BC28”, “BC29”, “BC30”, “BC31”, and “BC32”.

FIG. 18 shows SEC analysis of “BC28” and “BC30”, each following one-step purification using the CaptureSelect™ CH1 affinity resin.

FIG. 19 illustrates features of an exemplary bivalent bispecific binding molecule, “BC44”, further described in Example 5.

FIGS. 20A and 20B show size exclusion chromatography data of two bivalent binding molecules, “BC15” and “BC16”, respectively, under accelerated stability testing conditions. “BC15” and “BC16” have different variable region-CH3 junctions.

FIG. 21 presents a schematic of polypeptide chains and their domains, with respective naming conventions, for the trivalent 2×1 antibody constructs described herein.

FIG. 22 illustrates features of an exemplary trivalent 2×1 bispecific binding molecule, “BC1-2×1”, further described in Example 7.

FIG. 23 shows non-reducing SDS-PAGE of “BC1” and “BC1-2×1” protein expressed using the ThermoFisher Expi293 transient transfection system, at various stages of purification.

FIG. 24 compares the avidity of the bivalent 1×1 construct “BC1” to the avidity of the trivalent 2×1 construct “BC1-2×1” using an Octet (Pall ForteBio) biolayer interferometry analysis.

FIG. 25 illustrates salient features of a trivalent 2×1 construct, “TB111.”

FIG. 26 presents a schematic of polypeptide chains and their domains, with respective naming conventions, for the trivalent 1×2 antibody constructs described herein.

FIG. 27 illustrates features of an exemplary trivalent 1×2 construct “CTLA4-4×Nivo×CTLA4-4”, further described in Example 10.

FIG. 28 is a SDS-PAGE gel in which the lanes showing the trivalent 1×2 construct “CTLA4-4×Nivo×CTLA4-4” construct under non-reducing (“−DTT”) and reducing (“+DTT”) conditions have been boxed.

FIG. 29 shows a comparison of antigen binding between two antibodies: bivalent 1×1 construct “CTLA4-4×OX40-8” and the trivalent 1×2 construct “CTLA4-4×Nivo×CTLA4-4.” “CTLA4-4×OX40-8” binds to CTLA4 monovalently, while “CTLA4-4×Nivo×CTLA4-4” binds to CTLA4 bivalently.

FIG. 30 illustrates features of an exemplary trivalent 1×2 trispecific construct, “BC28-1×1×1a”, further described in Example 11.

FIG. 31 shows size exclusion chromatography of “BC28-1×1×1a” following transient expression and single step CH1 affinity resin purification, demonstrating a single well-defined peak.

FIG. 32 shows SDS-PAGE results with bivalent and trivalent constructs, each after transient expression and one-step purification using the CaptureSelect™ CH1 affinity resin, under non-reducing and reducing conditions, as further described in Example 12.

FIGS. 33A-33C show Octet binding analyses to 3 antigens: PD1, Antigen “A”, and CTLA4. As further described in Example 13, FIG. 33A shows binding of “BC1” to PD1 and Antigen “A”; FIG. 33B shows binding of a bivalent bispecific construct “CTLA4-4×OX40-8” to CTLA4, Antigen “A”, and PD1; FIG. 33C shows binding of trivalent trispecific “BC28-1×1×1a” to PD1, Antigen “A”, and CTLA4.

FIG. 34 presents a schematic of polypeptide chains and their domains, with respective naming conventions, for certain tetravalent 2×2 constructs described herein.

FIG. 35 illustrates certain salient features of the exemplary tetravalent 2×2 construct, “BC22-2×2” further described in Example 14.

FIG. 36 is a non-reducing SDS-PAGE gel comparing the 2×2 tetravalent “BC22-2×2” construct to a 1×2 trivalent construct “BC12-1×2” and a 2×1 trivalent construct “BC21-2×1” at different stages of purification.

FIG. 37 provides architecture for an exemplary tetravalent 2×2 construct.

FIG. 38 presents a schematic of polypeptide chains and their domains, with respective naming conventions, for certain tetravalent constructs described herein.

FIG. 39 provides exemplary architecture of a bispecific tetravalent construct.

FIG. 40 provides exemplary architecture for a trispecific tetravalent construct utilizing a common light chain strategy.

FIG. 41 shows bispecific antigen engagement by the tetravalent construct schematized in FIG. 39, demonstrating that this construct was capable of simultaneous engagement. The biolayer interferometry (BLI) response from B-Body immobilization and TNFα binding to the immobilized construct are consistent with a molecule with a high percentage of intended chain pairing.

FIG. 42 provides flow cytometry analysis of B-Body binding to cell-surface antigen. Cross-hatched signal indicates cells without antigen; dotted signal indicates transiently transfected cells with surface antigen.

FIG. 43 provides exemplary architecture of a trivalent construct.

FIG. 44 provides exemplary architecture of a trivalent construct.

FIG. 45 shows SDS-PAGE results with bivalent and trivalent constructs, each after transient expression and one-step purification using the CaptureSelect™ CH1 affinity resin, under non-reducing and reducing conditions, as further described in Example 17.

FIG. 46 shows differences in the thermal transitions for “BC24jv”, “BC26jv”, and “BC28jv” measured to assess pairing stability of junctional variants.

FIG. 47 demonstrates Octet (Pall ForteBio) biolayer interferometry analysis of a two-fold serial dilution (200-12.5 nM) used to determine binding affinity to CD3 for a non-mutagenized SP34-89 monovalent B-Body.

FIG. 48 shows SDS-PAGE analysis of bispecific antibodies comprising standard knob-hole orthogonal mutations introduced into CH3 domains found in their native positions within the Fc portion of the bispecific antibody that have been purified using a single-step CH1 affinity purification step (CaptureSelect™ CH1 affinity resin).

FIG. 49 shows Octet (Pall ForteBio) biolayer interferometry analysis demonstrating FcγRIa binding to trastuzumab (FIG. 49A “WT IgG1”), but not sFc10 (FIG. 49B).

FIG. 50 shows killing by trastuzumab (Herceptin, “WT-IgG1”) but not by the Fc variants tested in an ADCC assay.

FIG. 51 shows C1q binding by trastuzumab (Herceptin, “WT-IgG1”) but not by the Fc variants tested in a C1q ELISA.

FIG. 52 presents a schematic of polypeptide chains and their domains, with respective naming conventions, for a series of trivalent trispecific 2×1 antibody constructs described herein.

FIG. 53 presents a schematic of polypeptide chains and their domains, with respective naming conventions, for a series of trivalent trispecific 2×1 antibody constructs described herein.

FIG. 54 presents a schematic of polypeptide chains and their domains, with respective naming conventions, for a series of trivalent trispecific 1×2 antibody constructs described herein.

FIG. 55 presents a schematic of polypeptide chains and their domains, with respective naming conventions, for a series of trivalent trispecific 1×2 antibody constructs described herein.

FIG. 56 shows the Octet binding analysis for the VL domains paired with the OX40-13 VH domain, with non-cognate VL domains 1-12 and 21-24 shown in FIG. 56A, non-cognate VL domains 25-40 shown in FIG. 56B, and non-cognate VL domains 14-20 and cognate VL domain VL13 shown in FIG. 56C.

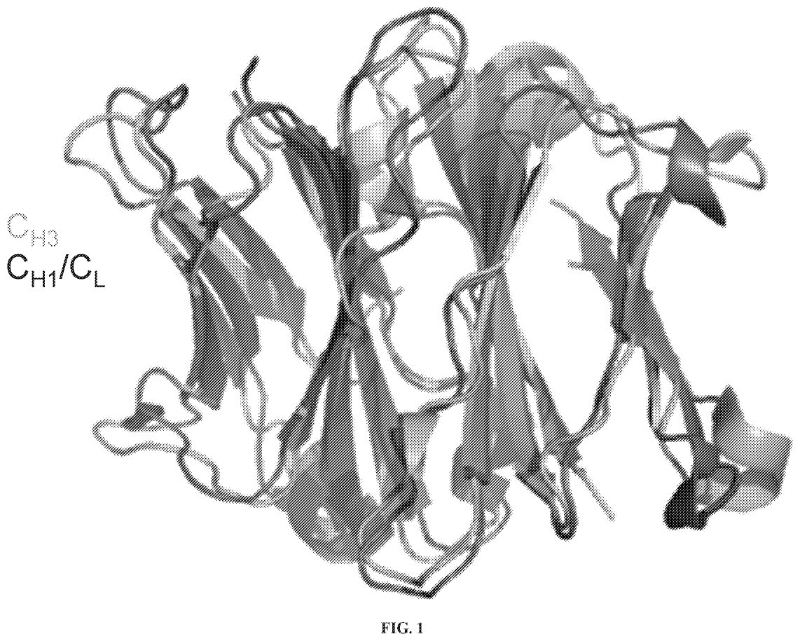

The figures depict various embodiments of the present invention for purposes of illustration only. One skilled in the art will readily recognize from the following discussion that alternative embodiments of the structures and methods illustrated herein may be employed without departing from the principles of the invention described herein.

### 6. DETAILED DESCRIPTION

### 6.1. Definitions

Unless defined otherwise, all technical and scientific terms used herein have the meaning commonly understood by a person skilled in the art to which this invention belongs. As used herein, the following terms have the meanings ascribed to them below.

By “antigen binding site” is meant a region of a trivalent trispecific binding molecule that specifically recognizes or binds to a given antigen or epitope.

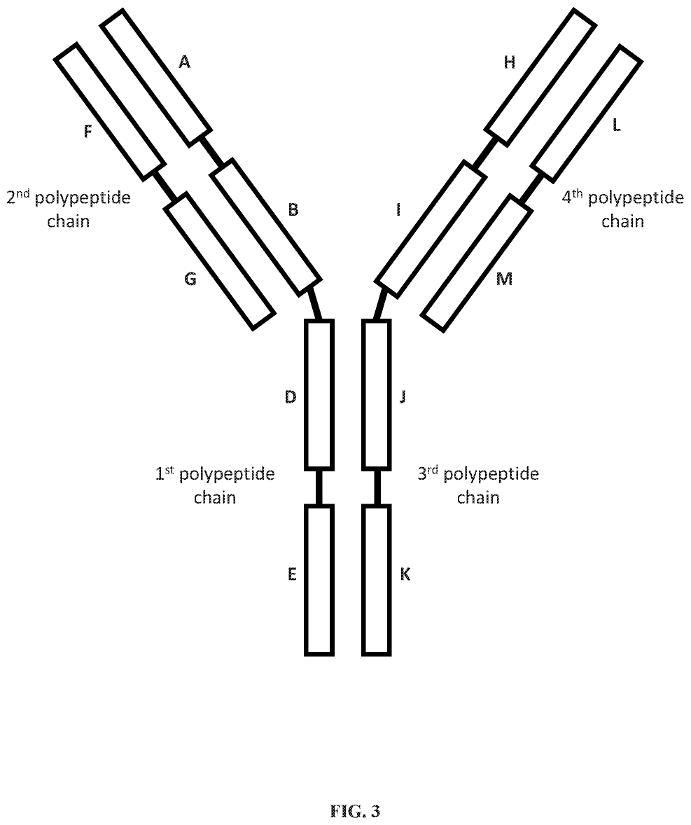

“B-Body,” as used herein and with reference to FIG. 3, refers to binding molecules comprising the features of a first and a second polypeptide chain, wherein: (a) the first polypeptide chain comprises a domain A, a domain B, a domain D, and a domain E, wherein the domains are arranged, from N-terminus to C-terminus, in a A-B-D-E orientation, and wherein domain A has a VL amino acid sequence, domain B has a CH3 amino acid sequence, domain D has a CH2 amino acid sequence, and domain E has a constant region domain amino acid sequence; (b) the second polypeptide chain comprises a domain F and a domain G, wherein the domains are arranged, from N-terminus to C-terminus, in a F-G orientation, and wherein domain F has a VH amino acid sequence and domain G has a CH3 amino acid sequence; and (c) the first and the second polypeptides are associated through an interaction between the A and the F domains and an interaction between the B and the G domains to form the binding molecule. B-bodies are described in more detail in International Patent Application No. PCT/US2017/057268, herein incorporated by reference in its entirety.

As used herein, the terms “treat” or “treatment” refer to both therapeutic treatment and prophylactic or preventative measures, wherein the object is to prevent or slow down (lessen) an undesired physiological change or disorder, such as the progression of multiple sclerosis, arthritis, or cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sports, or pet animals such as dogs, cats, guinea pigs, rabbits, rats, mice, horses, cattle, cows, and so on.

The term “sufficient amount” means an amount sufficient to produce a desired effect, e.g., an amount sufficient to modulate protein aggregation in a cell.

The term “therapeutically effective amount” is an amount that is effective to ameliorate a symptom of a disease. A therapeutically effective amount can be a “prophylactically effective amount” as prophylaxis can be considered therapy.

### 6.2. Other Interpretational Conventions

Unless otherwise specified, all references to sequences herein are to amino acid sequences.

Unless otherwise specified, antibody constant region residue numbering is according to the Eu index as described at
www.imgt.org/IMGTScientificChart/Numbering/Hu_IGHGnber.html#refs (accessed Aug. 22, 2017) and in Edelman et al., Proc. Natl. Acad. USA, 63:78-85 (1969), which are hereby incorporated by reference in their entireties, and identifies the residue according to its location in an endogenous constant region sequence regardless of the residue's physical location within a chain of the trivalent trispecific binding molecules described herein. By “endogenous sequence” or “native sequence” is meant any sequence, including both nucleic acid and amino acid sequences, which originates from an organism, tissue, or cell and has not been artificially modified or mutated.

Polypeptide chain numbers (e.g., a “first” polypeptide chains, a “second” polypeptide chain. etc. or polypeptide “chain 1,” “chain 2,” etc.) are used herein as a unique identifier for specific polypeptide chains that form a binding molecule and is not intended to connote order or quantity of the different polypeptide chains within the binding molecule.

In this disclosure, “comprises,” “comprising,” “containing,” “having,” “includes,” “including,” and linguistic variants thereof have the meaning ascribed to them in U.S. Patent law, permitting the presence of additional components beyond those explicitly recited.

Ranges provided herein are understood to be shorthand for all of the values within the range, inclusive of the recited endpoints. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

Unless specifically stated or apparent from context, as used herein the term “or” is understood to be inclusive. Unless specifically stated or apparent from context, as used herein, the terms “a”, “an”, and “the” are understood to be singular or plural.

Unless specifically stated or otherwise apparent from context, as used herein the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

### 6.3. Trivalent Trispecific Binding Molecules

In a first aspect, trivalent trispecific binding molecules are provided. The trivalent trispecific binding molecules have three antigen binding sites in which the ABSs collectively have three recognition specificities and are therefore termed “trivalent trispecific.”

### 6.3.1. Trivalent Trispecific 2×1 Antibody Architectures

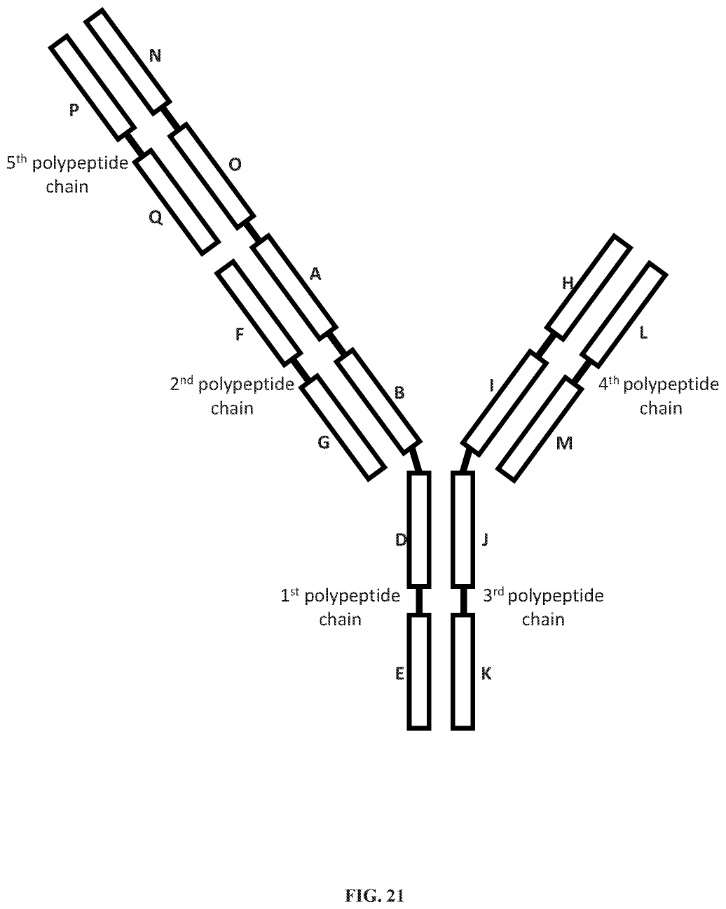

With reference to FIG. 21, in various trivalent embodiments the trivalent trispecific binding molecules comprises a first, a second, a third, a fourth, and a fifth polypeptide chain, wherein:

(a) the first polypeptide chain comprises a domain A, a domain B, a domain D, a domain E, a domain N and a domain O, wherein the domains are arranged, from N-terminus to C-terminus, in an N-O-A-B-D-E orientation, and domain A has a variable region domain amino acid sequence, domain B has a constant region domain amino acid sequence, domain D has a CH2 amino acid sequence, domain E has a constant region domain amino acid sequence, domain N has a variable region domain amino acid sequence, and domain O has a constant region domain amino acid sequence;

(b) the second polypeptide chain comprises a domain F and a domain G, wherein the domains are arranged, from N-terminus to C-terminus, in a F-G orientation, and wherein domain F has a variable region domain amino acid sequence and domain G has a constant region domain amino acid sequence amino acid sequence;

(c) the third polypeptide chain comprises a domain H, a domain I, a domain J, and a domain K, wherein the domains are arranged, from N-terminus to C-terminus, in a H-I-J-K orientation, and wherein domain H has a variable region domain amino acid sequence, domain I has a constant region domain amino acid sequence, domain J has a CH2 amino acid sequence, and K has a constant region domain amino acid sequence;

(d) the fourth polypeptide chain comprises a domain L and a domain M, wherein the domains are arranged, from N-terminus to C-terminus, in a L-M orientation, and wherein domain L has a variable region domain amino acid sequence and domain M has a constant region domain amino acid sequence;

(e) the fifth polypeptide chain comprises a domain P and a domain Q, wherein the domains are arranged, from N-terminus to C-terminus, in a P-Q orientation, and wherein domain P has a variable region domain amino acid sequence and domain Q has a constant region domain amino acid sequence,

(f) the first and the second polypeptides are associated through an interaction between the A and the F domains and an interaction between the B and the G domains;

(g) the third and the fourth polypeptides are associated through an interaction between the H and the L domains and an interaction between the I and the M domains;

(h) the first and the fifth polypeptides are associated through an interaction between the N and the P domains and an interaction between the O and the Q domains to form the binding molecule;

(i) the first and the third polypeptides are associated through an interaction between the D and the J domains and an interaction between the E and the K domains to form the binding molecule;

(j) the amino acid sequences of domain N, domain A, and domain H are different,

(k) the second and the fifth polypeptide chains are identical and the fourth polypeptide chain is different, or the fourth and the fifth polypeptide chains are identical and the second polypeptide chain is different; and

(l) the interaction between the A domain and the F domain form a first antigen binding site specific for a first antigen, the interaction between the H domain and the L domain form a second antigen binding site specific for a second antigen, and the interaction between the N domain and the P domain form a third antigen binding site specific for a third antigen.

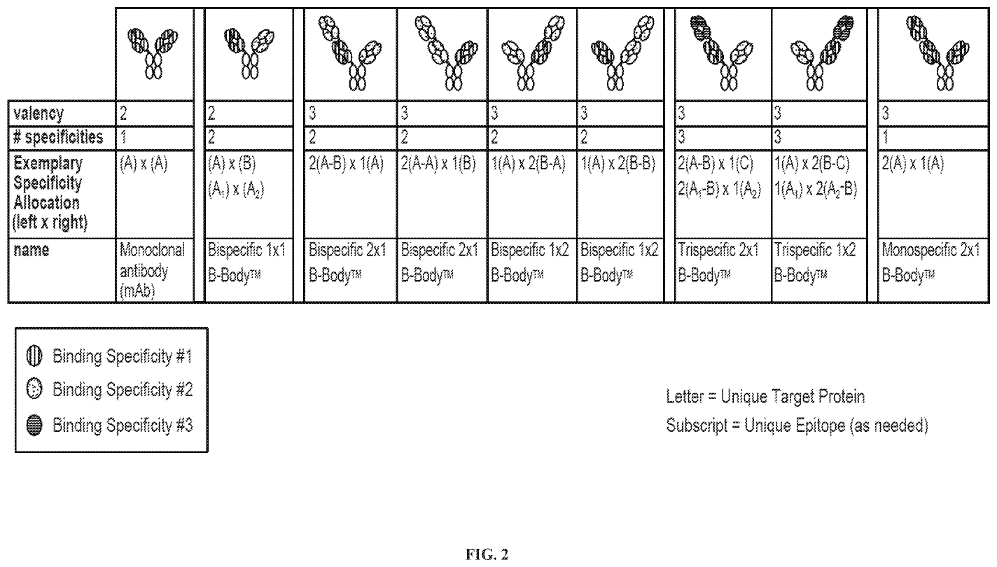

As schematized in FIG. 2, these trivalent embodiments are termed “2×1” trivalent constructs.

With reference to FIG. 52, in certain embodiments, the second and the fifth polypeptide chains are identical and the fourth polypeptide chain is different from the second and the fifth polypeptide chains, the amino acid sequences of domain O and domain B are identical, and the amino acid sequences of domain I is different from domains O and B.

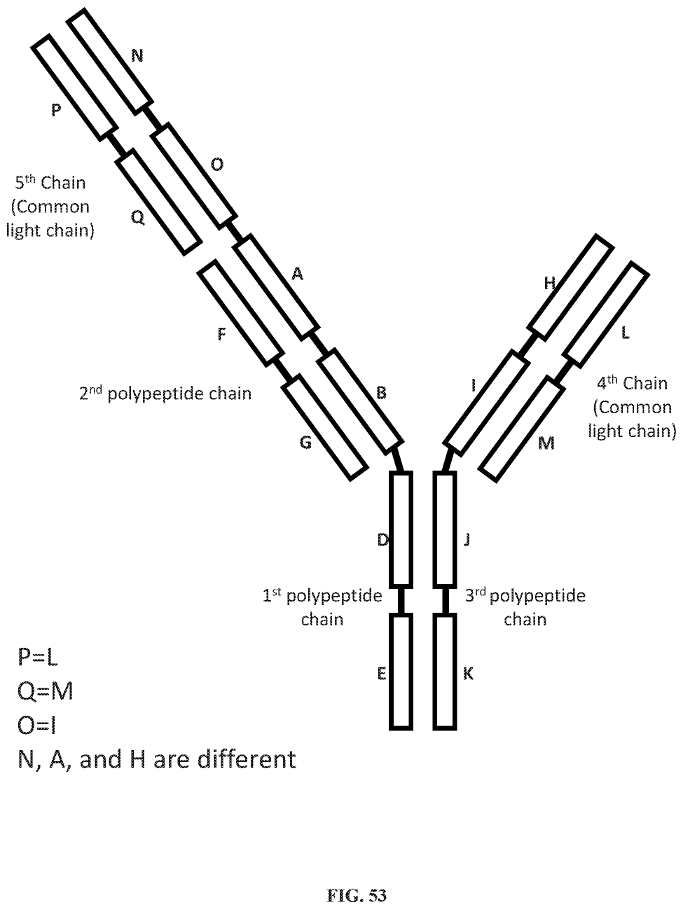

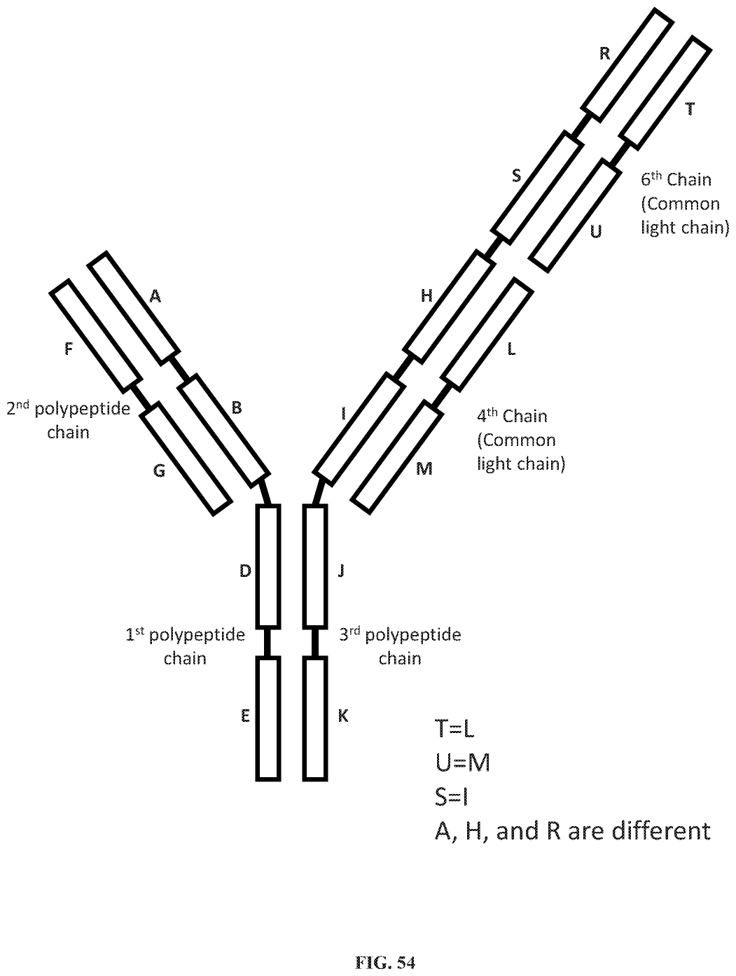

With reference to FIG. 53, in certain embodiments, the fourth and the fifth polypeptide chains are identical and the second polypeptide chain is different from the second and the fifth polypeptide chains, the amino acid sequences of domain O and domain I are identical, and the amino acid sequences of domain B is different from domains O and I.

In a variety of embodiments, the domain O is connected to domain A through a peptide linker. In a variety of embodiments, the domain S is connected to domain H through a peptide linker. In a preferred embodiment, the peptide linker connecting either domain O to domain A or connecting domain S to domain H is a 6 amino acid GSGSGS peptide sequence, as described in more detail in Section 6.3.20.6.

### 6.3.2. Trivalent Trispecific 1×2 Antibody Architectures

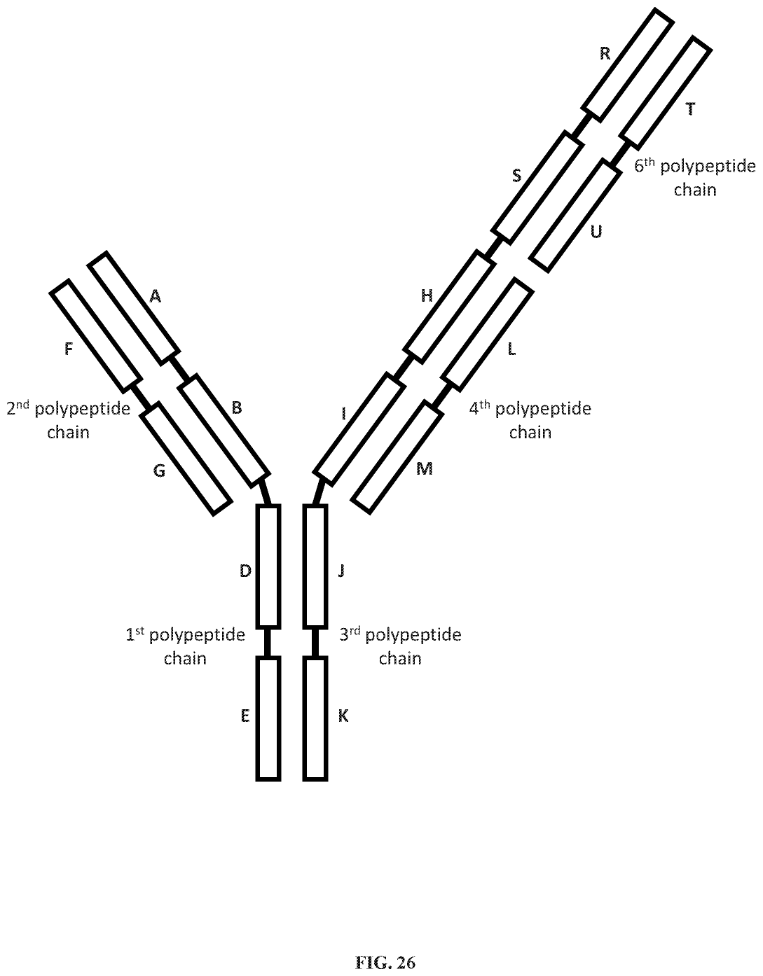

With reference to FIG. 26, in various trivalent embodiments the trivalent trispecific binding molecules comprises a first, a second, a third, a fourth, and a sixth polypeptide chain, wherein:

(a) the first polypeptide chain comprises a domain A, a domain B, a domain D, and a domain E, wherein the domains are arranged, from N-terminus to C-terminus, in an A-B-D-E orientation, and domain A has a variable region domain amino acid sequence, domain B has a constant region domain amino acid sequence, domain D has a CH2 amino acid sequence, and domain E has a constant region domain amino acid sequence;

(b) the second polypeptide chain comprises a domain F and a domain G, wherein the domains are arranged, from N-terminus to C-terminus, in a F-G orientation, and wherein domain F has a variable region domain amino acid sequence and domain G has a constant region domain amino acid sequence amino acid sequence;

(c) the third polypeptide chain comprises a domain H, a domain I, a domain J, a domain K, a domain R, and a domain S wherein the domains are arranged, from N-terminus to C-terminus, in a R-S-H-I-J-K orientation, and wherein domain H has a variable region domain amino acid sequence, domain I has a constant region domain amino acid sequence, domain J has a CH2 amino acid sequence, domain K has a constant region domain amino acid sequence, domain R has a variable region domain amino acid sequence, and domain S has a constant region domain amino acid sequence;

(d) the fourth polypeptide chain comprises a domain L and a domain M, wherein the domains are arranged, from N-terminus to C-terminus, in a L-M orientation, and wherein domain L has a variable region domain amino acid sequence and domain M has a constant region domain amino acid sequence;

(e) the sixth polypeptide chain comprises a domain T and a domain U, wherein the domains are arranged, from N-terminus to C-terminus, in a T-U orientation, and wherein domain T has a variable region domain amino acid sequence and domain U has a constant region domain amino acid sequence,

(f) the first and the second polypeptides are associated through an interaction between the A and the F domains and an interaction between the B and the G domains;

(g) the third and the fourth polypeptides are associated through an interaction between the H and the L domains and an interaction between the I and the M domains;

(h) the first and the sixth polypeptides are associated through an interaction between the R and the T domains and an interaction between the S and the U domains to form the binding molecule;

(i) the first and the third polypeptides are associated through an interaction between the D and the J domains and an interaction between the E and the K domains to form the binding molecule;

(j) the amino acid sequences of domain R, domain A, and domain H are different,

(k) the second and the sixth polypeptide chains are identical and the fourth polypeptide chain is different, or the fourth and the sixth polypeptide chains are identical and the second polypeptide chain is different, and

(l) the interaction between the A domain and the F domain form a first antigen binding site specific for a first antigen, the interaction between the H domain and the L domain form a second antigen binding site specific for a second antigen, and the interaction between the R domain and the T domain form a third antigen binding site specific for a third antigen.

As schematized in FIG. 2, these trivalent embodiments are termed “1×2” trivalent constructs.

With reference to FIG. 54, in certain embodiments, the fourth and the sixth polypeptide chains are identical and the fourth polypeptide chain is different from the second and the sixth polypeptide chains, the amino acid sequences of domain S and domain I are identical, and the amino acid sequences of domain B is different from domains S and I.

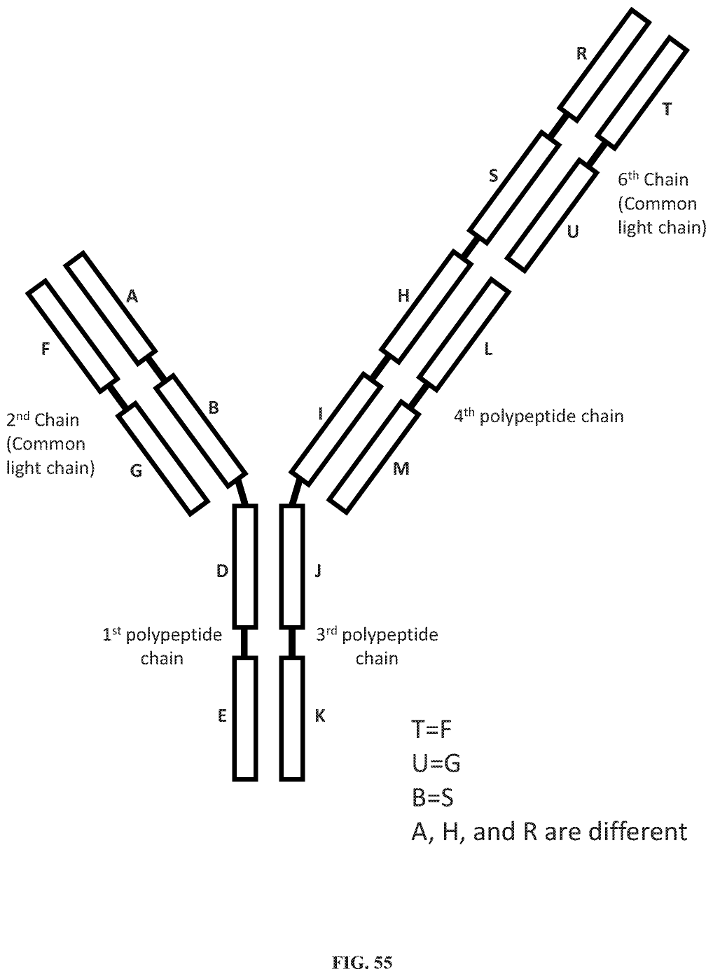

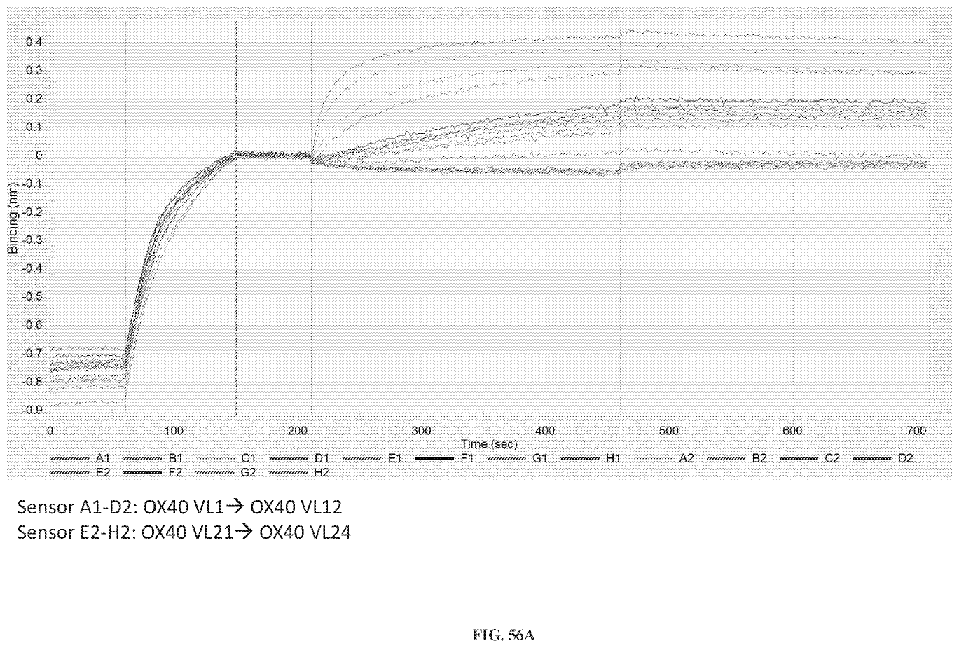

With reference to FIG. 55, in certain embodiments, the second and the sixth polypeptide chains are identical and the fourth polypeptide chain is different from the second and the sixth polypeptide chains, the amino acid sequences of domain S and domain B are identical, and the amino acid sequences of domain I is different from domains S and B.

In a variety of embodiments, the domain O is connected to domain A through a peptide linker. In a variety of embodiments, the domain S is connected to domain H through a peptide linker. In a preferred embodiment, the peptide linker connecting either domain O to domain A or connecting domain S to domain H is a 6 amino acid GSGSGS peptide sequence, as described in more detail in Section 6.3.20.6.

### 6.3.3. Domain A (Variable Region)

In the trivalent trispecific binding molecules, domain A has a variable region domain amino acid sequence. Variable region domain amino acid sequences, as described herein, are variable region domain amino acid sequences of an antibody including VL and VH antibody domain sequences. VL and VH sequences are described in greater detail below in Sections 6.3.3.1 and 6.3.3.4, respectively. In a preferred embodiment, domain A has a VL antibody domain sequence and domain F has a VH antibody domain sequence.

### 6.3.3.1.VL Regions

The VL amino acid sequences useful in the trivalent trispecific binding molecules described herein are antibody light chain variable domain sequences. In a typical arrangement in both natural antibodies and the antibody constructs described herein, a specific VL amino acid sequence associates with a specific VH amino acid sequence to form an antigen-binding site. In various embodiments, the VL amino acid sequences are mammalian sequences, including human sequences, synthesized sequences, or combinations of human, non-human mammalian, mammalian, and/or synthesized sequences, as described in further detail below in Sections 6.3.3.2 and 6.3.3.3.

In various embodiments, VL amino acid sequences are mutated sequences of naturally occurring sequences. In certain embodiments, the VL amino acid sequences are lambda (λ) light chain variable domain sequences. In certain embodiments, the VL amino acid sequences are kappa (κ) light chain variable domain sequences. In a preferred embodiment, the VL amino acid sequences are kappa (κ) light chain variable domain sequences.

In the trivalent trispecific binding molecules described herein, the C-terminus of domain A is connected to the N-terminus of domain B. In certain embodiments, domain A has a VL amino acid sequence that is mutated at its C-terminus at the junction between domain A and domain B, as described in greater detail below in Section 6.3.20.1 and in Example 6.

### 6.3.3.2. Complementarity Determining Regions

The VL amino acid sequences comprise highly variable sequences termed “complementarity determining regions” (CDRs), typically three CDRs (CDR1, CD2, and CDR3). In a variety of embodiments, the CDRs are mammalian sequences, including, but not limited to, mouse, rat, hamster, rabbit, camel, donkey, goat, and human sequences. In a preferred embodiment, the CDRs are human sequences. In various embodiments, the CDRs are naturally occurring sequences. In various embodiments, the CDRs are naturally occurring sequences that have been mutated to alter the binding affinity of the antigen-binding site for a particular antigen or epitope. In certain embodiments, the naturally occurring CDRs have been mutated in an in vivo host through affinity maturation and somatic hypermutation. In certain embodiments, the CDRs have been mutated in vitro through methods including, but not limited to, PCR-mutagenesis and chemical mutagenesis. In various embodiments, the CDRs are synthesized sequences including, but not limited to, CDRs obtained from random sequence CDR libraries and rationally designed CDR libraries.

### 6.3.3.3. Framework Regions and CDR Grafting

The VL amino acid sequences comprise “framework region” (FR) sequences. FRs are generally conserved sequence regions that act as a scaffold for interspersed CDRs (see Section 6.3.3.2.), typically in a FR1-CDR1-FR2-CDR2-FR3-CDR3-FR4 arrangement (from N-terminus to C-terminus). In a variety of embodiments, the FRs are mammalian sequences, including, but not limited to mouse, rat, hamster, rabbit, camel, donkey, goat, and human sequences. In a preferred embodiment, the FRs are human sequences. In various embodiments, the FRs are naturally occurring sequences. In various embodiments, the FRs are synthesized sequences including, but not limited, rationally designed sequences.

In a variety of embodiments, the FRs and the CDRs are both from the same naturally occurring variable domain sequence. In a variety of embodiments, the FRs and the CDRs are from different variable domain sequences, wherein the CDRs are grafted onto the FR scaffold with the CDRs providing specificity for a particular antigen. In certain embodiments, the grafted CDRs are all derived from the same naturally occurring variable domain sequence. In certain embodiments, the grafted CDRs are derived from different variable domain sequences. In certain embodiments, the grafted CDRs are synthesized sequences including, but not limited to, CDRs obtained from random sequence CDR libraries and rationally designed CDR libraries. In certain embodiments, the grafted CDRs and the FRs are from the same species. In certain embodiments, the grafted CDRs and the FRs are from different species. In a preferred grafted CDR embodiment, an antibody is “humanized”, wherein the grafted CDRs are non-human mammalian sequences including, but not limited to, mouse, rat, hamster, rabbit, camel, donkey, and goat sequences, and the FRs are human sequences. Humanized antibodies are discussed in more detail in U.S. Pat. No. 6,407,213, the entirety of which is hereby incorporated by reference for all it teaches. In various embodiments, portions or specific sequences of FRs from one species are used to replace portions or specific sequences of another species' FRs.

### 6.3.3.4.VH Regions

The VH amino acid sequences in the trivalent trispecific binding molecules described herein are antibody heavy chain variable domain sequences. In a typical antibody arrangement in both nature and in the trivalent trispecific binding molecules described herein, a specific VH amino acid sequence associates with a specific VL amino acid sequence to form an antigen-binding site. In various embodiments, VH amino acid sequences are mammalian sequences, including human sequences, synthesized sequences, or combinations of non-human mammalian, mammalian, and/or synthesized sequences, as described in further detail above in Sections 6.3.3.2 and 6.3.3.3. In various embodiments, VH amino acid sequences are mutated sequences of naturally occurring sequences.

### 6.3.4. Domain B (Constant Region)

In the trivalent trispecific binding molecules, Domain B has a constant region domain sequence. Constant region domain amino acid sequences, as described herein, are sequences of a constant region domain of an antibody.

In a variety of embodiments, the constant region sequences are mammalian sequences, including, but not limited to, mouse, rat, hamster, rabbit, camel, donkey, goat, and human sequences. In a preferred embodiment, the constant region sequences are human sequences. In certain embodiments, the constant region sequences are from an antibody light chain. In particular embodiments, the constant region sequences are from a lambda or kappa light chain. In certain embodiments, the constant region sequences are from an antibody heavy chain. In particular embodiments, the constant region sequences are an antibody heavy chain sequence that is an IgA1, IgA2, IgD, IgE, IgG1, IgG2, IgG3, IgG4, or IgM isotype. In a specific embodiment, the constant region sequences are from an IgG isotype. In a preferred embodiment, the constant region sequences are from an IgG1 isotype. In preferred specific embodiments, the constant region sequence is a CH3 sequence. CH3 sequences are described in greater detail below in Section 6.3.4.1. In other preferred embodiments, the constant region sequence is an orthologous CH2 sequence. Orthologous CH2 sequences are described in greater detail below in Section 6.3.4.2.

In particular embodiments, the constant region sequence has been mutated to include one or more orthogonal mutations. In a preferred embodiment, domain B has a constant region sequence that is a CH3 sequence comprising knob-hole (synonymously, “knob-in-hole,” “KIH”) orthogonal mutations, as described in greater detail below in Section 6.3.16.2, and either a S354C or a Y349C mutation that forms an engineered disulfide bridge with a CH3 domain containing an orthogonal mutation, as described in in greater detail below in Section 6.3.16.1. In some preferred embodiments, the knob-hole orthogonal mutation is a T366W mutation.

### 6.3.4.1.CH3 Regions

CH3 amino acid sequences, as described herein, are sequences of the C-terminal domain of an antibody heavy chain.

In a variety of embodiments, the CH3 sequences are mammalian sequences, including, but not limited to, mouse, rat, hamster, rabbit, camel, donkey, goat, and human sequences. In a preferred embodiment, the CH3 sequences are human sequences. In certain embodiments, the CH3 sequences are from an IgA1, IgA2, IgD, IgE, IgM, IgG1, IgG2, IgG3, IgG4 isotype or CH4 sequences from an IgE or IgM isotype. In a specific embodiment, the CH3 sequences are from an IgG isotype. In a preferred embodiment, the CH3 sequences are from an IgG1 isotype. In some embodiments, the CH3 sequence is from an IgA isotype.

In certain embodiments, the CH3 sequences are endogenous sequences. In particular embodiments, the CH3 sequence is UniProt accession number P01857 amino acids 224-330. In various embodiments, a CH3 sequence is a segment of an endogenous CH3 sequence. In particular embodiments, a CH3 sequence has an endogenous CH3 sequence that lacks the N-terminal amino acids G224 and Q225. In particular embodiments, a CH3 sequence has an endogenous CH3 sequence that lacks the C-terminal amino acids P328, G329, and K330. In particular embodiments, a CH3 sequence has an endogenous CH3 sequence that lacks both the N-terminal amino acids G224 and Q225 and the C-terminal amino acids P328, G329, and K330. In preferred embodiments, a trivalent trispecific binding molecule has multiple domains that have CH3 sequences, wherein a CH3 sequence can refer to both a full endogenous CH3 sequence as well as a CH3 sequence that lacks N-terminal amino acids, C-terminal amino acids, or both.

In certain embodiments, the CH3 sequences are endogenous sequences that have one or more mutations. In particular embodiments, the mutations are one or more orthogonal mutations that are introduced into an endogenous CH3 sequence to guide specific pairing of specific CH3 sequences, as described in more detail in Sections 6.3.16.1-6.3.16.4.

In certain embodiments, the CH3 sequences are engineered to reduce immunogenicity of the antibody by replacing specific amino acids of one allotype with those of another allotype and referred to herein as isoallotype mutations, as described in more detail in Stickler et al. (Genes Immun. 2011 April; 12(3): 213-221), which is herein incorporated by reference for all that it teaches. In particular embodiments, specific amino acids of the G1m1 allotype are replaced. In a preferred embodiment, isoallotype mutations D356E and L358M are made in the CH3 sequence.

In a preferred embodiment, domain B has a human IgG1 CH3 amino acid sequence with the following mutational changes: P343V; Y349C; and a tripeptide insertion, 445P, 446G, 447K. In other preferred embodiments, domain B has a human IgG1 CH3 sequence with the following mutational changes: T366K; and a tripeptide insertion, 445K, 446S, 447C. In still other preferred embodiments, domain B has a human IgG1 CH3 sequence with the following mutational changes: Y349C and a tripeptide insertion, 445P, 446G, 447K.

In certain embodiments, domain B has a human IgG1 CH3 sequence with a 447C mutation incorporated into an otherwise endogenous CH3 sequence.

In the trivalent trispecific binding molecules described herein, the N-terminus of domain B is connected to the C-terminus of domain A. In certain embodiments, domain B has a CH3 amino acid sequence that is mutated at its N-terminus at the junction between domain A and domain B, as described in greater detail below in Section 6.3.20.1 and Example 6.

In the trivalent trispecific binding molecules, the C-terminus of domain B is connected to the N-terminus of domain D. In certain embodiments, domain B has a CH3 amino acid sequence that is extended at the C-terminus at the junction between domain B and domain D, as described in greater detail below in Section 6.3.20.3.

In some embodiments, domain B comprises a human IgA CH3 sequence. IgA CH3 isotype substitution is described in greater detail in Section 6.3.16.4. An exemplary human IgA CH3 amino acid sequence is:

(SEQ ID NO: 84)
TFRPEVHLLPPPSEELALNELVTLTCLARGFSPKDVLVRWLQGSQELPRE
KYLTWASRQEPSQGTTTFAVTSILRVAAEDWKKGDTFSCMVGHEALPLAF
TQKTIDRL

### 6.3.4.2. Orthologous CH2 Regions

CH2 amino acid sequences, as described herein, are sequences of the third domain of an antibody heavy chain, with reference from the N-terminus to C-terminus. CH2 amino acid sequences, in general, are discussed in more detail below in section 6.3.5. In a series of embodiments, a trivalent trispecific binding molecule has more than one paired set of CH2 domains that have CH2 sequences, wherein a first set has CH2 amino acid sequences from a first isotype and one or more orthologous sets of CH2 amino acid sequences from another isotype. The orthologous CH2 amino acid sequences, as described herein, are able to interact with CH2 amino acid sequences from a shared isotype, but not significantly interact with the CH2 amino acid sequences from another isotype present in the trivalent trispecific binding molecule. In particular embodiments, all sets of CH2 amino acid sequences are from the same species. In preferred embodiments, all sets of CH2 amino acid sequences are human CH2 amino acid sequences. In other embodiments, the sets of CH2 amino acid sequences are from different species. In particular embodiments, the first set of CH2 amino acid sequences is from the same isotype as the other non-CH2 domains in the trivalent trispecific binding molecule. In a specific embodiment, the first set has CH2 amino acid sequences from an IgG isotype and the one or more orthologous sets have CH2 amino acid sequences from an IgM or IgE isotype. In certain embodiments, one or more of the sets of CH2 amino acid sequences are endogenous CH2 sequences. In other embodiments, one or more of the sets of CH2 amino acid sequences are endogenous CH2 sequences that have one or more mutations. In particular embodiments, the one or more mutations are orthogonal knob-hole mutations, orthogonal charge-pair mutations, or orthogonal hydrophobic mutations. Orthologous CH2 amino acid sequences useful for the trivalent trispecific binding molecules are described in more detail in international PCT applications WO2017/011342 and WO2017/106462, herein incorporated by reference in their entirety.

### 6.3.5. Domain D (Constant Region)

In the trivalent trispecific binding molecules described herein, domain D has a constant region amino acid sequence. Constant region amino acid sequences are described in more detail in Section 6.3.4.

In a preferred series of embodiments, domain D has a CH2 amino acid sequence. CH2 amino acid sequences, as described herein, are CH2 amino acid sequences of the third domain of a native antibody heavy chain, with reference from the N-terminus to C-terminus. In a variety of embodiments, the CH2 sequences are mammalian sequences, including but not limited to mouse, rat, hamster, rabbit, camel, donkey, goat, and human sequences. In a preferred embodiment, the CH2 sequences are human sequences. In certain embodiments, the CH2 sequences are from an IgA1, IgA2, IgD, IgE, IgG1, IgG2, IgG3, IgG4, or IgM isotype. In a preferred embodiment, the CH2 sequences are from an IgG1 isotype.

In certain embodiments, the CH2 sequences are endogenous sequences. In particular embodiments, the sequence is UniProt accession number P01857 amino acids 111-223. In a preferred embodiment, the CH2 sequences have an N-terminal hinge region peptide that connects the N-terminal variable domain-constant domain segment to the CH2 domain, as discussed in more detail below in Section 6.3.20.3.

In the trivalent trispecific binding molecules, the N-terminus of domain D is connected to the C-terminus of domain B. In certain embodiments, domain B has a CH3 amino acid sequence that is extended at the C-terminus at the junction between domain D and domain B, as described in greater detail below in Section 6.3.20.3.

### 6.3.6. Domain E (Constant Region)

In the trivalent trispecific binding molecules, domain E has a constant region domain amino acid sequence. Constant region amino acid sequences are described in more detail in Section 6.3.4.

In certain embodiments, the constant region sequence is a CH3 sequence. CH3 sequences are described in greater detail above in Section 6.3.4.1. In particular embodiments, the constant region sequence has been mutated to include one or more orthogonal mutations. In a preferred embodiment, domain E has a constant region sequence that is a CH3 sequence comprising knob-hole (synonymously, “knob-in-hole,” “KIH”) orthogonal mutations, as described in greater detail below in Section 6.3.16.2, and either a S354C or a Y349C mutation that forms an engineered disulfide bridge with a CH3 domain containing an orthogonal mutation, as described in in greater detail below in Section 6.3.16.1. In some preferred embodiments, the knob-hole orthogonal mutation is a T366W mutation.

In certain embodiments, the constant region domain sequence is a CH1 sequence. In particular embodiments, the CH1 amino acid sequence of domain E is the only CH1 amino acid sequence in the trivalent trispecific binding molecule. In certain embodiments, the N-terminus of the CH1 domain is connected to the C-terminus of a CH2 domain, as described in greater detail below in 6.3.20.5. In certain embodiments, the constant region sequence is a CL sequence. In certain embodiments, the N-terminus of the CL domain is connected to the C-terminus of a CH2 domain, as described in greater detail below in 6.3.20.5. CH1 and CL sequences are described in further detail in Section 6.3.10.1.

### 6.3.7. Domain F (Variable Region)

In the trivalent trispecific binding molecules, domain F has a variable region domain amino acid sequence. Variable region domain amino acid sequences, as discussed in greater detail in Section 6.3.1, are variable region domain amino acid sequences of an antibody including VL and VH antibody domain sequences. VL and VH sequences are described in greater detail above in Sections 6.3.3.1 and 6.3.3.4, respectively. In a preferred embodiment, domain F has a VH antibody domain sequence.

### 6.3.8. Domain G (Constant Region)

In the trivalent trispecific binding molecules, domain G has a constant region amino acid sequence. Constant region amino acid sequences are described in more detail in Section 6.3.4.

In preferred specific embodiments, the constant region sequence is a CH3 sequence. CH3 sequences are described in greater detail below in Section 6.3.4.1. In other preferred embodiments, the constant region sequence is an orthologous CH2 sequence. Orthologous CH2 sequences are described in greater detail below in Section 6.3.4.2.

In certain preferred embodiments, domain G has a human IgG1 CH3 sequence with the following mutational changes: S354C; and a tripeptide insertion, 445P, 446G, 447K. In some preferred embodiments, domain G has a human IgG1 CH3 sequence with the following mutational changes: S354C; and 445P, 446G, 447K tripeptide insertion. In some preferred embodiments, domain G has a human IgG1 CH3 sequence with the following changes: L351D, and a tripeptide insertion of 445G, 446E, 447C.

### 6.3.9. Domain H (Variable Region)

In the trivalent trispecific binding molecules, domain L has a variable region domain amino acid sequence. Variable region domain amino acid sequences, as discussed in greater detail in Section 6.3.1, are variable region domain amino acid sequences of an antibody including VL and VH antibody domain sequences. VL and VH sequences are described in greater detail above in Sections 6.3.3.1. and 6.3.3.4, respectively. In a preferred embodiment, domain H has a VL antibody domain sequence.

### 6.3.10. Domain I (Constant Region)

In the trivalent trispecific binding molecules, domain I has a constant region domain amino acid sequence. Constant region domain amino acid sequences are described in greater detail above in Section 6.3.4. In a series of preferred embodiments of the trivalent trispecific binding molecules, domain I has a CL amino acid sequence. In another series of embodiments, domain I has a CH1 amino acid sequence. CH1 and CL amino acid sequences are described in further detail in Section 6.3.10.1.

### 6.3.10.1. CH1 and CL Regions

CH1 amino acid sequences, as described herein, are sequences of the second domain of an antibody heavy chain, with reference from the N-terminus to C-terminus. In certain embodiments, the CH1 sequences are endogenous sequences. In a variety of embodiments, the CH1 sequences are mammalian sequences, including, but not limited to mouse, rat, hamster, rabbit, camel, donkey, goat, and human sequences. In a preferred embodiment, the CH1 sequences are human sequences. In certain embodiments, the CH1 sequences are from an IgA1, IgA2, IgD, IgE, IgG1, IgG2, IgG3, IgG4, or IgM isotype. In a preferred embodiment, the CH1 sequences are from an IgG1 isotype. In preferred embodiments, the CH1 sequence is UniProt accession number P01857 amino acids 1-98.

The CL amino acid sequences useful in the trivalent trispecific binding molecules described herein are antibody light chain constant domain sequences. In certain embodiments, the CL sequences are endogenous sequences. In a variety of embodiments, the CL sequences are mammalian sequences, including, but not limited to mouse, rat, hamster, rabbit, camel, donkey, goat, and human sequences. In a preferred embodiment, CL sequences are human sequences.

In certain embodiments, the CL amino acid sequences are lambda (λ) light chain constant domain sequences. In particular embodiments, the CL amino acid sequences are human lambda light chain constant domain sequences. In preferred embodiments, the lambda (λ) light chain sequence is UniProt accession number P0CG04.

In certain embodiments, the CL amino acid sequences are kappa (κ) light chain constant domain sequences. In a preferred embodiment, the CL amino acid sequences are human kappa (κ) light chain constant domain sequences. In a preferred embodiment, the kappa light chain sequence is UniProt accession number P01834.

In certain embodiments, the CH1 sequence and the CL sequences are both endogenous sequences. In certain embodiments, the CH1 sequence and the CL sequences separately comprise respectively orthogonal modifications in endogenous CH1 and CL sequences, as discussed below in greater detail in Section 6.3.10.2. It is to be understood that orthogonal mutations in the CH1 sequence do not eliminate the specific binding interaction between the CH1 binding reagent and the CH1 domain. However, in some embodiments, the orthogonal mutations may reduce, though not eliminate, the specific binding interaction. CH1 and CL sequences can also be portions thereof, either of an endogenous or modified sequence, such that a domain having the CH1 sequence, or portion thereof, can associate with a domain having the CH1 sequence, or portion thereof. Furthermore, the trivalent trispecific binding molecule having a portion of the CH1 sequences described above can be bound by the CH1 binding reagent.

Without wishing to be bound by theory, the CH1 domain is also unique in that it's folding is typically the rate limiting step in the secretion of IgG (Feige et al. Mol Cell. 2009 Jun. 12; 34(5):569-79; herein incorporated by reference in its entirety). Thus, purifying the trivalent trispecific binding molecules based on the rate limiting component of CH1 comprising polypeptide chains can provide a means to purify complete complexes from incomplete chains, e.g., purifying complexes having a limiting CH1 domain from complexes only having one or more non-CH1 comprising chains.

While the CH1 limiting expression may be a benefit in some aspects, as discussed, there is the potential for CH1 to limit overall expression of the complete trispecific trivalent binding molecules. Thus, in certain embodiments, the expression of the polypeptide chain comprising the CH1 sequence(s) is adjusted to improve the efficiency of the trivalent trispecific binding molecules forming complete complexes. In an illustrative example, the ratio of a plasmid vector constructed to express the polypeptide chain comprising the CH1 sequence(s) can be increased relative to the plasmid vectors constructed to express the other polypeptide chains. In another illustrative example, the polypeptide chain comprising the CH1 sequence(s) when compared to the polypeptide chain comprising the CL sequence(s) can be the smaller of the two polypeptide chains. In another specific embodiment, the expression of the polypeptide chain comprising the CH1 sequence(s) can be adjusted by controlling which polypeptide chain has the CH1 sequence(s). For example, engineering the trivalent trispecific binding molecule such that the CH1 domain is present in a two-domain polypeptide chain (e.g., the 4^{th} polypeptide chain described herein), instead of the CH1 sequence's native position in a four-domain polypeptide chain (e.g., the 3^{rd} polypeptide chain described herein), can be used to control the expression of the polypeptide chain comprising the CH1 sequence(s). However, in other aspects, a relative expression level of CH1 containing chains that is too high compared to the other chains can result in incomplete complexes the have the CH1 chain, but not each of the other chains. Thus, in certain embodiments, the expression of the polypeptide chain comprising the CH1 sequence(s) is adjusted to both reduce the formation incomplete complexes without the CH1 containing chain, and to reduce the formation incomplete complexes with the CH1 containing chain but without the other chains present in a complete complex.

### 6.3.10.2. C111 and CL Orthogonal Modifications

In certain embodiments, the CH1 sequence and the CL sequences separately comprise respectively orthogonal modifications in endogenous CH1 and CL sequences.

“Orthogonal modifications” or synonymously “orthogonal mutations” as described herein are one or more engineered mutations in an amino acid sequence of an antibody domain that alter the affinity of binding of a first domain having orthogonal modification for a second domain having a complementary orthogonal modification, as compared to binding of the first and second domains in the absence of the orthogonal modifications. In some embodiments, the orthogonal modifications decrease the affinity of binding of the first domain having the orthogonal modification for the second domain having the complementary orthogonal modification, as compared to binding of the first and second domains in the absence of the orthogonal modifications. In preferred embodiments, the orthogonal modifications increase the affinity of binding of the first domain having the orthogonal modification for the second domain having the complementary orthogonal modification, as compared to binding of the first and second domains in the absence of the orthogonal modifications. In certain preferred embodiments, the orthogonal modifications decrease the affinity of a domain having the orthogonal modifications for a domain lacking the complementary orthogonal modifications.

In certain embodiments, orthogonal modifications are mutations in an endogenous antibody domain sequence. In a variety of embodiments, orthogonal modifications are modifications of the N-terminus or C-terminus of an endogenous antibody domain sequence including, but not limited to, amino acid additions or deletions. In particular embodiments, orthogonal modifications include, but are not limited to, engineered disulfide bridges, knob-in-hole mutations, and charge-pair mutations, as described in greater detail below. In particular embodiments, orthogonal modifications include a combination of orthogonal modifications selected from, but not limited to, engineered disulfide bridges, knob-in-hole mutations, and charge-pair mutations. In particular embodiments, the orthogonal modifications can be combined with amino acid substitutions that reduce immunogenicity, such as isoallotype mutations as described in greater detail in Section 6.3.4.1.

In certain embodiments, the CH1 sequence and the CL sequence of the CH1/CL pair separately comprise respectively orthogonal modifications in endogenous CH1 and CL sequences. In other embodiments, one sequence of the CH1/CL pair comprises at least one modification while the other sequence of the CH1/CL pair does not comprise a modification in the respectively orthogonal amino acid position.

A CH1/CL orthogonal modification may affect the CH1/CL domain pairing via an interaction between a modified residue in the CH1 domain and a corresponding modified or unmodified residue in the CL domain.

It is to be understood that orthogonal mutations in the CH1 sequence do not eliminate the specific binding interaction between the CH1 binding reagent and the CH1 domain. However, in some embodiments, the orthogonal mutations may reduce, though not eliminate, the specific binding interaction. CH1 and CL sequences can also be portions thereof, either of an endogenous or modified sequence, such that a domain having the CH1 sequence, or portion thereof, can associate with a domain having the CH1 sequence, or portion thereof. Furthermore, the binding molecule having a portion of the CH1 sequences described herein can be bound by the CH1 binding reagent.

### Exemplary CH1/CL Orthogonal Modifications: Engineered Disulfide Bridges

Some embodiments of a CH1/CL orthogonal modification comprise an engineered disulfide bridge between engineered cysteines in CH1 and CL. Such engineered disulfide bridges may stabilize an interaction between the polypeptide comprising the modified CH1 and the polypeptide comprising the corresponding modified CL.

An orthogonal CH1/CL modification comprising an engineered disulfide bridge can comprise, by way of example only, a CH1 domain having an engineered cysteine at position 128, 129, 138, 141, 168, or 171, as numbered by the EU index. Such an orthogonal CH1/CL modification comprising an engineered disulfide bridge may further comprise, by way of example only, a CL domain having an engineered cysteine at position 116, 118, 119, 164, 162, or 210, as numbered by the EU index.

For example, a CH1/CL orthogonal modification may be selected from engineered cysteines at position 138 of the CH1 sequence and position 116 of the CL sequence, at position 128 of the CH1 sequence and position 119 of the CL sequence, or at position 129 of the CH1 sequence and position 210 of the CL sequence, as numbered and discussed in more detail in U.S. Pat. Nos. 8,053,562 and 9,527,927, each incorporated herein by reference in its entirety. In some embodiments, the CH1/CL orthogonal modification comprises an engineered cysteine at position 141 of the CH1 sequence and position 118 of the CL sequence, as numbered by the EU index.

In some embodiments, the CH1/CL orthogonal modification comprises an engineered cysteine at position 168 of the CH1 sequence and position 164 of the CL sequence, as numbered by the EU index. In some embodiments, the CH1/CL orthogonal modification comprises an engineered cysteine at position 128 of the CH1 sequence and position 118 of the CL sequence, as numbered by the EU index. In some embodiments, the CH1/CL orthogonal modification comprises an engineered cysteine at position 171 of the CH1 sequence and position 162 of the CL sequence, as numbered by the EU index. In some embodiments, the CL sequence is a CL-lambda sequence. In preferred embodiments, the CL sequence is a CL-kappa sequence. In some embodiments, the engineered cysteines are at position 128 of the CH1 sequence and position 118 of the CL Kappa sequence, as numbered by the EU index.

Table 8 below provides exemplary CH1/CL orthogonal modifications comprising an engineered disulfide bridge between CH1 and CL, numbered according to the EU index.

TABLE 8
Exemplary CH1/CL engineered disulfide bridges

| CH1 mutation | CL mutation |
|---|---|
| A141C | F118C |
| H168C | T164C |
| L128C | F118C |
| P171C | S162C |

In a series of preferred embodiments, the mutations that provide non-endogenous (engineered) cysteine amino acids are a F118C mutation in the CL sequence with a corresponding A141C in the CH1 sequence, or a F118C mutation in the CL sequence with a corresponding L128C in the CH1 sequence, a T164C mutation in the CL sequence with a corresponding H168C mutation in the CH1 sequence, or a S162C mutation in the CL sequence with a corresponding P171C mutation in the CH1 sequence, as numbered by the Eu index.

### CH1/CL Orthogonal Modifications: Charged-Pair Mutations

In a variety of embodiments, the orthogonal modifications in the CL sequence and the CH1 sequence are charge-pair mutations. As used herein, charge-pair mutations are amino acid substitutions that affect the charge of a residue in a domain's surface such that the domain will preferentially associate with a second domain having complementary charge-pair mutations relative to association with domains without the complementary charge-pair mutations. In certain embodiments, charge-pair mutations improve orthogonal association between specific domains. Charge-pair mutations are described in greater detail in U.S. Pat. Nos. 8,592,562, 9,248,182, and 9,358,286, each of which is incorporated by reference herein for all they teach. In certain embodiments, charge-pair mutations improve stability between specific domains. In specific embodiments the charge-pair mutations are a F118S, F118A or F118V mutation in the CL sequence with a corresponding A141L in the CH1 sequence, or a T129R mutation in the CL sequence with a corresponding K147D in the CH1 sequence, as numbered by the Eu index and described in greater detail in Bonisch et al. (Protein Engineering, Design & Selection, 2017, pp. 1-12), herein incorporated by reference for all that it teaches.

In some cases, the CH1/CL charge-pair mutations are a N138K mutation in the CL sequence with a corresponding G166D in the CH1 sequence, or a N138D mutation in the CL sequence with a corresponding G166K in the CH1 sequence, as numbered by the Eu index. In some embodiments, the charge-pair mutations are a P127E mutation in CH1 sequence with a corresponding E123K mutation in the corresponding C1 sequence. In some embodiments, the charge-pair mutations are a P127K mutation in CH1 sequence with a corresponding E123 (not mutated) in the corresponding CL sequence.

Table 9 below provides exemplary CH1/CL orthogonal charged-pair modifications.

TABLE 9
Exemplary CH1/CL orthogonal charged-pair modifications

| CH1 mutation | CL mutation |
|---|---|
| G166D | N138K |
| G166D | N138D |
| G166K | N138K |
| G166K | N138D |
| P127E | E123K |
| P127E | No mutation (E123) |
| P127K | E123K |
| P127K | No mutation (E123) |

### 6.3.10.3. Combinations of CH1/CL Orthogonal Modifications

In certain embodiments, the CH1 and CL domains of a single CH1/CL pair separately contain two or more respectively orthogonal modifications in endogenous CH1 and CL sequences. For instance, the CH1 and CL sequence may contain a first orthogonal modification and a second orthogonal modification in the endogenous CH1 and CL sequences. The two or more respectively orthogonal modifications in endogenous CH1 and CL sequences can be selected from any of the CH1/CL orthogonal modifications described herein.

In some embodiments, the first orthogonal modification is an orthogonal charge-pair mutation, and the second orthogonal modification is an orthogonal engineered disulfide bridge. In some embodiments, the first orthogonal modification is an orthogonal charge-pair mutation as described in Table 9, and the additional orthogonal modification comprise an engineered disulfide bridge selected from engineered cysteines at position 138 of the CH1 sequence and position 116 of the CL sequence, at position 128 of the CH1 sequence and position 119 of the CL sequence, or at position 129 of the CH1 sequence and position 210 of the CL sequence, as numbered and discussed in more detail in U.S. Pat. Nos. 8,053,562 and 9,527,927, each incorporated herein by reference in its entirety. In some embodiments, the first orthogonal modification is an orthogonal charge-pair mutation as described in Table 9, and the additional orthogonal modification comprise an engineered disulfide bridge as described in Table 8. In some embodiments, the first orthogonal modification comprises an L128C mutation in the CH1 sequence and an F118C mutation in the CL sequence, and the second orthogonal modification comprises a modification of residue 166 in the same CH1 sequence and a modification of residue 138 in the same CL sequence. In some embodiments, the first orthogonal modification comprises an L128C mutation in the CH1 sequence and an F118C mutation in the CL sequence, and the second orthogonal modification comprises a G166D mutation in the CH1 sequence and a N138K mutation in the CL sequence. In some embodiments, the first orthogonal modification comprises an L128C mutation in the CH1 sequence and an F118C mutation in the CL sequence, and the second orthogonal modification comprises a G166K mutation in the CH1 sequence and a N138D mutation in the CL sequence.

### 6.3.11. Domain J (CH2)

In the trivalent trispecific binding molecules, domain J has a CH2 amino acid sequence. CH2 amino acid sequences are described in greater detail above in Section 6.3.5. In a preferred embodiment, the CH2 amino acid sequence has an N-terminal hinge region that connects domain J to domain I, as described in more detail below in Section 6.3.20.4.

In the trivalent trispecific binding molecules, the C-terminus of domain J is connected to the N-terminus of domain K. In particular embodiments, domain J is connected to the N-terminus of domain K that has a CH1 amino acid sequence or CL amino acid sequence, as described in further detail below in Section 6.3.20.5.

### 6.3.12. Domain K (Constant Region)

In the trivalent trispecific binding molecules, domain K has a constant region domain amino acid sequence. Constant region domain amino acid sequences are described in greater detail above in Section 6.3.4. In a preferred embodiment, domain K has a constant region sequence that is a CH3 sequence comprising knob-hole orthogonal mutations, as described in greater detail below in Section 6.3.16.2; isoallotype mutations, as described in more detail above in 6.3.4.1; and either a S354C or a Y349C mutation that forms an engineered disulfide bridge with a CH3 domain containing an orthogonal mutation, as described in in greater detail below in Section 6.3.16.1. In some preferred embodiments, the knob-hole orthogonal mutations combined with isoallotype mutations are the following mutational changes: D356E, L358M, T366S, L368A, and Y407V.

In certain embodiments, the constant region domain sequence is a CH1 sequence. In particular embodiments, the CH1 amino acid sequence of domain K is the only CH1 amino acid sequence in the trivalent trispecific binding molecule. In certain embodiments, the N-terminus of the CH1 domain is connected to the C-terminus of a CH2 domain, as described in greater detail below in 6.3.20.5. In certain embodiments, the constant region sequence is a CL sequence. In certain embodiments, the N-terminus of the CL domain is connected to the C-terminus of a CH2 domain, as described in greater detail below in 6.3.20.5. CH1 and CL sequences are described in further detail in Section 6.3.10.1.

### 6.3.13. Domain L (Variable Region)

In the trivalent trispecific binding molecules, domain L has a variable region domain amino acid sequence. Variable region domain amino acid sequences, as discussed in greater detail in Section 6.3.1, are variable region domain amino acid sequences of an antibody including VL and VH antibody domain sequences. VL and VH sequences are described in greater detail above in Sections 6.3.3.1. and 6.3.3.4, respectively. In a preferred embodiment, domain L has a VH antibody domain sequence.

### 6.3.14. Domain M (Constant Region)

In the trivalent trispecific binding molecules, domain M has a constant region domain amino acid sequence. Constant region domain amino acid sequences are described in greater detail above in Section 6.3.4. In a series of preferred embodiments of the trivalent trispecific binding molecules, domain I has a CH1 amino acid sequence. In another series of preferred embodiments, domain I has a CL amino acid sequence. CH1 and CL amino acid sequences are described in further detail in Section 6.3.10.1.

### 6.3.15. Pairing of Domains A & F

In the trivalent trispecific binding molecules, a domain A VL or VH amino acid sequence and a cognate domain F VL or VH amino acid sequence are associated and form an antigen binding site (ABS). The A:F antigen binding site (ABS) is capable of specifically binding an epitope of an antigen. Antigen binding by an ABS is described in greater detail below in Section 6.3.15.1.

In a variety of multivalent embodiments, the ABS formed by domains A and F (A:F) is identical in sequence to one or more other ABSs within the trivalent trispecific binding molecule and therefore has the same recognition specificity as the one or more other sequence-identical ABSs within the trivalent trispecific binding molecule.

In a variety of multivalent embodiments, the A:F ABS is non-identical in sequence to one or more other ABSs within the trivalent trispecific binding molecule. In certain embodiments, the A:F ABS has a recognition specificity different from that of one or more other sequence-non-identical ABSs in the trivalent trispecific binding molecule. In particular embodiments, the A:F ABS recognizes a different antigen from that recognized by at least one other sequence-non-identical ABS in the trivalent trispecific binding molecule. In particular embodiments, the A:F ABS recognizes a different epitope of an antigen that is also recognized by at least one other sequence-non-identical ABS in the trivalent trispecific binding molecule. In these embodiments, the ABS formed by domains A and F recognizes an epitope of antigen, wherein one or more other ABSs within the trivalent trispecific binding molecule recognizes the same antigen but not the same epitope.

### 6.3.15.1. Binding of Antigen by ABS

An ABS, and the trivalent trispecific binding molecule comprising such ABS, is said to “recognize” the epitope (or more generally, the antigen) to which the ABS specifically binds, and the epitope (or more generally, the antigen) is said to be the “recognition specificity” or “binding specificity” of the ABS.

The ABS is said to bind to its specific antigen or epitope with a particular affinity. As described herein, “affinity” refers to the strength of interaction of non-covalent intermolecular forces between one molecule and another. The affinity, i.e. the strength of the interaction, can be expressed as a dissociation equilibrium constant (K_{D}), wherein a lower K_{D} value refers to a stronger interaction between molecules. K_{D} values of antibody constructs are measured by methods well known in the art including, but not limited to, bio-layer interferometry (e.g. Octet/FORTEBIO®, surface plasmon resonance (SPR) technology (e.g. Biacore®), and cell binding assays. For purposes herein, affinities are dissociation equilibrium constants measured by bio-layer interferometry using Octet/FORTEBIO®.

“Specific binding,” as used herein, refers to an affinity between an ABS and its cognate antigen or epitope in which the K_{D} value is below 10⁻⁶M, 10⁻⁷M, 10⁻⁸M, 10⁻⁹M, or 10^{-m}M.

The number of ABSs in a binding molecule as described herein defines the “valency” of the binding molecule, as schematized in FIG. 2. A binding molecule having a single ABS is “monovalent”. A binding molecule having a plurality of ABSs is said to be “multivalent”. A multivalent binding molecule having two ABSs is “bivalent.” A multivalent binding molecule having three ABSs is “trivalent.” A multivalent binding molecule having four ABSs is “tetravalent.”

In various multivalent embodiments, all of the plurality of ABSs have the same recognition specificity. As schematized in FIG. 2, such a binding molecule is a “monospecific” “multivalent” binding construct. In other multivalent embodiments, at least two of the plurality of ABSs have different recognition specificities. Such binding molecules are multivalent and “multispecific”. In multivalent embodiments in which the ABSs collectively have two recognition specificities, the binding molecule is “bispecific.” In multivalent embodiments in which the ABSs collectively have three recognition specificities, the binding molecule is “trispecific.”

In multivalent embodiments in which the ABSs collectively have a plurality of recognition specificities for different epitopes present on the same antigen, the binding molecule is “multiparatopic.” Multivalent embodiments in which the ABSs collectively recognize two epitopes on the same antigen are “biparatopic.”

In various multivalent embodiments, multivalency of the binding molecule, including the trivalent trispecific binding molecules described herein, improves the avidity of the binding molecule for a specific target. As described herein, “avidity” refers to the overall strength of interaction between two or more molecules, e.g. a multivalent binding molecule for a specific target, wherein the avidity is the cumulative strength of interaction provided by the affinities of multiple ABSs. Avidity can be measured by the same methods as those used to determine affinity, as described above. In certain embodiments, the avidity of a trivalent trispecific binding molecule for a specific target is such that the interaction is a specific binding interaction, wherein the avidity between two molecules has a K_{D} value below 10⁻⁶M, 10⁻⁷M, 10⁻⁸M, 10⁻⁹M, or 10⁻¹° M. In certain embodiments, the avidity of a binding molecule for a specific target has a K_{D} value such that the interaction is a specific binding interaction, wherein the one or more affinities of individual ABSs do not have has a K_{D} value that qualifies as specifically binding their respective antigens or epitopes on their own. In certain embodiments, the avidity is the cumulative strength of interaction provided by the affinities of multiple ABSs for separate antigens on a shared specific target or complex, such as separate antigens found on an individual cell. In certain embodiments, the avidity is the cumulative strength of interaction provided by the affinities of multiple ABSs for separate epitopes on a shared individual antigen.

### 6.3.16. Pairing of Domains B & G

In the trivalent trispecific binding molecules described herein, a domain B constant region amino acid sequence and a domain G constant region amino acid sequence are associated. Constant region domain amino acid sequences are described in greater detail above in Section 6.3.4.

In a series of preferred embodiments, domain B and domain G have CH3 amino acid sequences. CH3 sequences are described in greater detail above in Section 6.3.4.1. In various embodiments, the amino acid sequences of the B and the G domains are identical. In certain of these embodiments, the sequence is an endogenous CH3 sequence.

In a variety of embodiments, the amino acid sequences of the B and the G domains are different, and separately comprise respectively orthogonal modifications in an endogenous CH3 sequence, wherein the B domain interacts with the G domain, and wherein neither the B domain nor the G domain significantly interacts with a CH3 domain lacking the orthogonal modification.

“Orthogonal modifications” or synonymously “orthogonal mutations” as described herein are one or more engineered mutations in an amino acid sequence of an antibody domain that increase the affinity of binding of a first domain having orthogonal modification for a second domain having a complementary orthogonal modification. In certain embodiments, the orthogonal modifications decrease the affinity of a domain having the orthogonal modifications for a domain lacking the complementary orthogonal modifications. In certain embodiments, orthogonal modifications are mutations in an endogenous antibody domain sequence. In a variety of embodiments, orthogonal modifications are modifications of the N-terminus or C-terminus of an endogenous antibody domain sequence including, but not limited to, amino acid additions or deletions. In particular embodiments, orthogonal modifications include, but are not limited to, engineered disulfide bridges, knob-in-hole mutations, and charge-pair mutations, and isotype substitution as described in greater detail in Sections 6.3.16.1-6.3.16.4. In particular embodiments, orthogonal modifications include a combination of orthogonal modifications selected from, but not limited to, engineered disulfide bridges, knob-in-hole mutations, and charge-pair mutations. In particular embodiments, the orthogonal modifications can be combined with amino acid substitutions that reduce immunogenicity, such as isoallotype mutations, as described in greater detail above in Section 6.3.4.1.

### 6.3.16.1. Orthogonal Engineered Disulfide Bridges

In a variety of embodiments, the orthogonal modifications comprise mutations that generate engineered disulfide bridges between a first and a second domain. As described herein, “engineered disulfide bridges” are mutations that provide non-endogenous cysteine amino acids in two or more domains such that a non-native disulfide bond forms when the two or more domains associate. Engineered disulfide bridges are described in greater detail in Merchant et al. (Nature Biotech (1998) 16:677-681), the entirety of which is hereby incorporated by reference for all it teaches. In certain embodiments, engineered disulfide bridges improve orthogonal association between specific domains. In a particular embodiment, the mutations that generate engineered disulfide bridges are a K392C mutation in one of a first or second CH3 domains, and a D399C in the other CH3 domain. In a preferred embodiment, the mutations that generate engineered disulfide bridges are a S354C mutation in one of a first or second CH3 domains, and a Y349C in the other CH3 domain. In another preferred embodiment, the mutations that generate engineered disulfide bridges are a 447C mutation in both the first and second CH3 domains that are provided by extension of the C-terminus of a CH3 domain incorporating a KSC tripeptide sequence.

### 6.3.16.2. Orthogonal Knob-Hole Mutations

In a variety of embodiments, orthogonal modifications comprise knob-hole (synonymously, knob-in-hole) mutations. As described herein, knob-hole mutations are mutations that change the steric features of a first domain's surface such that the first domain will preferentially associate with a second domain having complementary steric mutations relative to association with domains without the complementary steric mutations. Knob-hole mutations are described in greater detail in U.S. Pat. Nos. 5,821,333 and 8,216,805, each of which is incorporated herein in its entirety. In various embodiments, knob-hole mutations are combined with engineered disulfide bridges, as described in greater detail in Merchant et al. (Nature Biotech (1998) 16:677-681)), incorporated herein by reference in its entirety. In various embodiments, knob-hole mutations, isoallotype mutations, and engineered disulfide mutations are combined.

In certain embodiments, the knob-in-hole mutations are a T366Y mutation in a first domain, and a Y407T mutation in a second domain. In certain embodiments, the knob-in-hole mutations are a F405A in a first domain, and a T394W in a second domain. In certain embodiments, the knob-in-hole mutations are a T366Y mutation and a F405A in a first domain, and a T394W and a Y407T in a second domain. In certain embodiments, the knob-in-hole mutations are a T366W mutation in a first domain, and a Y407A in a second domain. In certain embodiments, the combined knob-in-hole mutations and engineered disulfide mutations are a S354C and T366W mutations in a first domain, and a Y349C, T366S, L368A, and aY407V mutation in a second domain. In a preferred embodiment, the combined knob-in-hole mutations, isoallotype mutations, and engineered disulfide mutations are a S354C and T366W mutations in a first domain, and a Y349C, D356E, L358M, T366S, L368A, and aY407V mutation in a second domain.

### 6.3.16.3. Orthogonal Charge-Pair Mutations

In a variety of embodiments, orthogonal modifications are charge-pair mutations. As used herein, charge-pair mutations are mutations that affect the charge of an amino acid in a domain's surface such that the domain will preferentially associate with a second domain having complementary charge-pair mutations relative to association with domains without the complementary charge-pair mutations. In certain embodiments, charge-pair mutations improve orthogonal association between specific domains. Charge-pair mutations are described in greater detail in U.S. Pat. Nos. 8,592,562, 9,248,182, and 9,358,286, each of which is incorporated by reference herein for all they teach. In certain embodiments, charge-pair mutations improve stability between specific domains. In a preferred embodiment, the charge-pair mutations are a T366K mutation in a first domain, and a L351D mutation in the other domain.

### 6.3.16.4. IgA-CH3 Isotype Domain Substitution

In some embodiments, it is desirable to reduce an undesired association of a first and second domain, which may contain CH3 sequences, with a third and fourth domain, which may also contain CH3 sequences. In such cases, use of CH3 sequences from human IgA (IgA-CH3) in the first and/or second domain may improve antibody assembly and stability by reducing such undesired associations. In some embodiments of a binding molecule wherein the third and fourth domain comprise IgG-CH3 sequences, the first and/or second domain comprises IgA-CH3 sequences.

In some embodiments, at least one of the first or second domain comprise a CH3 linker sequence as described in Section 6.3.20.3. In some embodiments, both the first and second domain comprise a CH3 linker sequence as described in Section 6.3.20.3. In some embodiments, the first comprises a first CH3 linker sequence and the second domain comprises a second CH3 linker sequence. In some embodiments, the first CH3 linker sequence associates with the second CH3 linker sequence by formation of a disulfide bridge between cysteine residues of the first and second CH3 linker sequences. In some embodiments, the first CH3 linker and the second CH3 linker are identical. In some embodiments, the first CH3 linker and second CH3 linker are non-identical. In some embodiments, the first CH3 linker and second CH3 linker differ in length by 1-6 amino acids. In some embodiments, the first CH3 linker and second CH3 linker differ in length by 1-3 amino acids.

In some embodiments, the first CH3 linker and the second CH3 linker are provided in Table 10 below.

TABLE 10
CH3 linker sequences for BAvariants

| BA | First CH3 | Second CH3 |
|---|---|---|
| variant # | linker sequence | linker sequence |
| BA1 | GEC | GEC |
| BA2 | AGC | AGKGSC |
| BA3 | AGKGC | AGC |
| BA4 | AGKGSC | AGC |
| BA5 | AGKC | AGC |
| BA9 | AGC | AGC |
| BA10 | AGC | AGKGC |
| BA11 | AGC | AGKGSC |
| BA12 | AGC | AGKC |
| BA13 | AGC | GEC |
| BA14 | AGC | PGKC |
| BA15 | AGKGC | AGC |
| BA16 | AGKGC | AGKGC |
| BA17 | AGKGC | AGKGSC |
| BA18 | AGKGC | AGKC |
| BA19 | AGKGC | GEC |
| BA20 | AGKGC | PGKC |
| BA21 | AGKGSC | AGC |
| BA22 | AGKGSC | AGKGC |
| BA23 | AGKGSC | AGKGSC |
| BA24 | AGKGSC | AGKC |
| BA25 | AGKGSC | GEC |
| BA26 | AGKGSC | PGKC |
| BA27 | AGKC | AGC |
| BA28 | AGKC | AGKGC |
| BA29 | AGKC | AGKGSC |
| BA30 | AGKC | AGKC |
| BA31 | AGKC | GEC |
| BA32 | AGKC | PGKC |
| BA33 | GEC | AGC |
| BA34 | GEC | AGKGC |
| BA35 | GEC | AGKGSC |
| BA36 | GEC | AGKC |
| BA37 | GEC | GEC |
| BA38 | GEC | PGKC |
| BA39 | PGKC | AGC |
| BA40 | PGKC | AGKGC |
| BA41 | PGKC | AGKGSC |
| BA42 | PGKC | AGKC |
| BA43 | PGKC | GEC |
| BA44 | PGKC | PGKC |

In preferred embodiments, the first CH3 linker is AGC and the second CH3 linker is AGKGSC. In some embodiments, the first CH3 linker is AGKGC and the second CH3 linker is AGC. In some embodiments, the first CH3 linker is AGKGSC and the second CH3 linker is AGC. In some embodiments, the first CH3 linker is AGKC and the second CH3 linker is AGC.

### 6.3.17. Pairing of Domains E & K

In various embodiments, the E domain has a CH3 amino acid sequence.

In various embodiments, the K domain has a CH3 amino acid sequence.

In a variety of embodiments, the amino acid sequences of the E and K domains are identical, wherein the sequence is an endogenous CH3 sequence.

In a variety of embodiments, the sequences of the E and K domains are different. In a variety of embodiments, the different sequences separately comprise respectively orthogonal modifications in an endogenous CH3 sequence, wherein the E domain interacts with the K domain, and wherein neither the E domain nor the K domain significantly interacts with a CH3 domain lacking the orthogonal modification. In certain embodiments, the orthogonal modifications include, but are not limited to, engineered disulfide bridges, knob-in-hole mutations, charge-pair mutations, and isotype substitution as described in greater detail in Sections 6.3.16.1-6.3.16.4. In particular embodiments, orthogonal modifications include a combination of orthogonal modifications selected from, but not limited to, engineered disulfide bridges, knob-in-hole mutations, and charge-pair mutations. In particular embodiments, the orthogonal modifications can be combined with amino acid substitutions that reduce immunogenicity, such as isoallotype mutations.

### 6.3.18. Pairing of Domains I & M and Domains H & L

In a variety of embodiments, domain I has a CL sequence and domain M has a CH1 sequence. In a variety of embodiments, domain H has a VL sequence and domain L has a VH sequence. In a preferred embodiment, domain H has a VL amino acid sequence, domain I has a CL amino acid sequence, domain L has a VH amino acid sequence, and domain M has a CH1 amino acid sequence. In another preferred embodiment, domain H has a VL amino acid sequence, domain I has a CL amino acid sequence, domain L has a VH amino acid sequence, domain M has a CH1 amino acid sequence, and domain K has a CH3 amino acid sequence.

In a variety of embodiments, the amino acid sequences of the I domain and the M domain separately comprise respectively orthogonal modifications in an endogenous sequence, wherein the I domain interacts with the M domain, and wherein neither the I domain nor the M domain significantly interacts with a domain lacking the orthogonal modification. In a series of embodiments, the orthogonal mutations in the I domain are in a CL sequence and the orthogonal mutations in the M domain are in CH1 sequence. Orthogonal mutations are in CH1 and CL sequences are described in more detail above in Section 6.3.10.2.

In a variety of embodiments, the amino acid sequences of the H domain and the L domain separately comprise respectively orthogonal modifications in an endogenous sequence, wherein the H domain interacts with the L domain, and wherein neither the H domain nor the L domain significantly interacts with a domain lacking the orthogonal modification. In a series of embodiments, the orthogonal mutations in the H domain are in a VL sequence and the orthogonal mutations in the L domain are in VH sequence. In specific embodiments, the orthogonal mutations are charge-pair mutations at the VH/VL interface. In preferred embodiments, the charge-pair mutations at the VH/VL interface are a Q39E in VH with a corresponding Q38K in VL, or a Q39K in VH with a corresponding Q38E in VL, as described in greater detail in Igawa et al. (Protein Eng. Des. Sel., 2010, vol. 23, 667-677), herein incorporated by reference for all it teaches.

In certain embodiments, the interaction between the A domain and the F domain form a first antigen binding site specific for a first antigen, and the interaction between the H domain and the L domain form a second antigen binding site specific for a second antigen. In certain embodiments, the interaction between the A domain and the F domain form a first antigen binding site specific for a first antigen, and the interaction between the H domain and the L domain form a second antigen binding site specific for the first antigen.

### 6.3.19. Tetravalent 2×2 Binding Molecules

In a variety of embodiments, the binding molecules have 4 antigen binding sites and are therefore termed “tetravalent.”

With reference to FIG. 34, in a further series of embodiments, the binding molecules further comprise a fifth and a sixth polypeptide chain, wherein (a) the first polypeptide chain further comprises a domain N and a domain O, wherein the domains are arranged, from N-terminus to C-terminus, in a N-O-A-B-D-E orientation; (b) the third polypeptide chain further comprises a domain R and a domain S, wherein the domains are arranged, from N-terminus to C-terminus, in a R-S-H-I-J-K orientation; (c) the binding molecule further comprises a fifth and a sixth polypeptide chain, wherein the fifth polypeptide chain comprises a domain P and a domain Q, wherein the domains are arranged, from N-terminus to C-terminus, in a P-Q orientation, and the sixth polypeptide chain comprises a domain T and a domain U, wherein the domains are arranged, from N-terminus to C-terminus, in a T-U orientation; and (d) the first and the fifth polypeptides are associated through an interaction between the N and the P domains and an interaction between the O and the Q domains, and the third and the sixth polypeptides are associated through an interaction between the R and the T domains and an interaction between the S and the U domains to form the binding molecule.

In a variety of embodiments, the domain O is connected to domain A through a peptide linker and the domain S is connected to domain H through a peptide linker. In a preferred embodiment, the peptide linker connecting domain O to domain A and connecting domain S to domain H is a 6 amino acid GSGSGS peptide sequence, as described in more detail in Section 6.3.20.6.

### 6.3.19.1. Tetravalent 2×2 Bispecific Constructs

With reference to FIG. 34, in a series of tetravalent 2×2 bispecific binding molecules, the amino acid sequences of domain N and domain A are identical, the amino acid sequences of domain H and domain R are identical, the amino acid sequences of domain O and domain B are identical, the amino acid sequences of domain I and domain S are identical, the amino acid sequences of domain P and domain F are identical, the amino acid sequences of domain L and domain T are identical, the amino acid sequences of domain Q and domain G are identical, the amino acid sequences of domain M and domain U are identical; and wherein the interaction between the A domain and the F domain form a first antigen binding site specific for a first antigen, the domain N and domain P form a second antigen binding site specific for the first antigen, the interaction between the H domain and the L domain form a third antigen binding site specific for a second antigen, and the interaction between the R domain and the T domain form a fourth antigen binding site specific for the second antigen.

With reference to FIG. 34, in another series of tetravalent 2×2 bispecific binding molecules, the amino acid sequences of domain H and domain A are identical, the amino acid sequences of domain N and domain R are identical, the amino acid sequences of domain I and domain B are identical, the amino acid sequences of domain O and domain S are identical, the amino acid sequences of domain L and domain F are identical, the amino acid sequences of domain P and domain T are identical, the amino acid sequences of domain M and domain G are identical, the amino acid sequences of domain Q and domain U are identical; and wherein the interaction between the A domain and the F domain form a first antigen binding site specific for a first antigen, the domain N and domain P form a second antigen binding site specific for a second antigen, the interaction between the H domain and the L domain form a third antigen binding site specific for the first antigen, and the interaction between the R domain and the T domain form a fourth antigen binding site specific for the second antigen.

### 6.3.20. Domain Junctions

### 6.3.20.1. Junctions Connecting VL and CH3 Domains

In a variety of embodiments, the amino acid sequence that forms a junction between the C-terminus of a VL domain and the N-terminus of a CH3 domain is an engineered sequence. In certain embodiments, one or more amino acids are deleted or added in the C-terminus of the VL domain. In certain embodiments, the junction connecting the C-terminus of a VL domain and the N-terminus of a CH3 domain is one of the sequences described in Table 2 below in Section 6.13.7. In particular embodiments, A111 is deleted in the C-terminus of the VL domain. In certain embodiments, one or more amino acids are deleted or added in the N-terminus of the CH3 domain. In particular embodiments, P343 is deleted in the N-terminus of the CH3 domain. In particular embodiments, P343 and R344 are deleted in the N-terminus of the CH3 domain. In certain embodiments, one or more amino acids are deleted or added to both the C-terminus of the VL domain and the N-terminus of the CH3 domain. In particular embodiments, A111 is deleted in the C-terminus of the VL domain and P343 is deleted in the N-terminus of the CH3 domain. In a preferred embodiment, A111 and V110 are deleted in the C-terminus of the VL domain. In another preferred embodiment, A111 and V110 are deleted in the C-terminus of the VL domain and the N-terminus of the CH3 domain has a P343V mutation.

### 6.3.20.2. Junctions Connecting VH and CH3 Domains

In a variety of embodiments, the amino acid sequence that forms a junction between the C-terminus of a VH domain and the N-terminus of a CH3 domain is an engineered sequence. In certain embodiments, one or more amino acids are deleted or added in the C-terminus of the VH domain. In certain embodiments, the junction connecting the C-terminus of a VH domain and the N-terminus of the CH3 domain is one of the sequences described in Table 3 below in Section 6.13.7. In particular embodiments, K117 and G118 are deleted in the C-terminus of the VH domain. In certain embodiments, one or more amino acids are deleted or added in the N-terminus of the CH3 domain. In particular embodiments, P343 is deleted in the N-terminus of the CH3 domain. In particular embodiments, P343 and R344 are deleted in the N-terminus of the CH3 domain. In particular embodiments, P343, R344, and E345 are deleted in the N-terminus of the CH3 domain. In certain embodiments, one or more amino acids are deleted or added to both the C-terminus of the VH domain and the N-terminus of the CH3 domain. In a preferred embodiment, T116, K117, and G118 are deleted in the C-terminus of the VH domain.

### 6.3.20.3. Junctions Connecting CH3 C-Terminus to CH2 N-Terminus (Hinge)

In the trivalent trispecific binding molecules described herein, the N-terminus of the CH2 domain has a “hinge” region amino acid sequence. As used herein, hinge regions are sequences of an antibody heavy chain that link the N-terminal variable domain-constant domain segment of an antibody and a CH2 domain of an antibody. In addition, the hinge region typically provides both flexibility between the N-terminal variable domain-constant domain segment and CH2 domain, as well as amino acid sequence motifs that form disulfide bridges between heavy chains (e.g. the first and the third polypeptide chains). As used herein, the hinge region amino acid sequence is SEQ ID NO: 56.

In a variety of embodiments, a CH3 amino acid sequence is extended at the C-terminus at the junction between the C-terminus of the CH3 domain and the N-terminus of a CH2 domain. In certain embodiments, a CH3 amino acid sequence is extended at the C-terminus at the junction between the C-terminus of the CH3 domain and a hinge region, which in turn is connected to the N-terminus of a CH2 domain. In a preferred embodiment, the CH3 amino acid sequence is extended by inserting a PGK tripeptide sequence followed by the DKTHT motif of an IgG1 hinge region.

In a particular embodiment, the extension at the C-terminus of the CH3 domain incorporates amino acid sequences that can form a disulfide bond with orthogonal C-terminal extension of another CH3 domain. In a preferred embodiment, the extension at the C-terminus of the CH3 domain incorporates a KSC tripeptide sequence that is followed by the DKTHT motif of an IgG1 hinge region that forms a disulfide bond with orthogonal C-terminal extension of another CH3 domain that incorporates a GEC motif of a kappa light chain.

### 6.3.20.4. Junctions Connecting CL C-Terminus and CH2 N-Terminus (Hinge)

In a variety of embodiments, a CL amino acid sequence is connected through its C-terminus to a hinge region, which in turn is connected to the N-terminus of a CH2 domain. Hinge region sequences are described in more detail above in Section 6.3.20.3. In a preferred embodiment, the hinge region amino acid sequence is SEQ ID NO:56.

### 6.3.20.5. Junctions Connecting CH2 C-Terminus to Constant Region Domain

In a variety of embodiments, a CH2 amino acid sequence is connected through its C-terminus to the N-terminus of a constant region domain. Constant regions are described in more detail above in Section 6.3.6. In a preferred embodiment, the CH2 sequence is connected to a CH3 sequence via its endogenous sequence. In other embodiments, the CH2 sequence is connected to a CH1 or CL sequence. Examples discussing connecting a CH2 sequence to a CH1 or CL sequence are described in more detail in U.S. Pat. No. 8,242,247, which is hereby incorporated in its entirety.

### 6.3.20.6. Junctions Connecting Domain O to Domain A or Domain S to Domain H on Trivalent and Tetravalent Molecules

In a variety of embodiments, heavy chains of antibodies (e.g. the first and third polypeptide chains) are extended at their N-terminus to include additional domains that provide additional ABSs. With reference to FIG. 21, FIG. 26, and FIG. 34, in certain embodiments, the C-terminus of the constant region domain amino acid sequence of a domain O and/or a domain S is connected to the N-terminus of the variable region domain amino acid sequence of a domain A and/or a domain H, respectively. In some preferred embodiments, the constant region domain is a CH3 amino acid sequence and the variable region domain is a VL amino acid sequence. In some preferred embodiments, the constant region domain is a CL amino acid sequence and the variable region domain is a VL amino acid sequence. In certain embodiments, the constant region domain is connected to the variable region domain through a peptide linker. In a preferred embodiment, the peptide linker is a 6 amino acid GSGSGS peptide sequence.

In a variety of embodiments, light chains of antibodies (e.g. the second and fourth polypeptide chains) are extended at their N-terminus to include additional variable domain-constant domain segments of an antibody. In certain embodiments, the constant region domain is a CH1 amino acid sequence and the variable region domain is a VH amino acid sequence.

### 6.4. Specific Bivalent B-Body Architectures

In a further aspect, trivalent trispecific binding molecules are provided that are based on the bivalent B-body architectures described below and in Sections 6.4.1-6.4.5.

With reference to FIG. 3, in a series of embodiments the bivalent B-body architectures comprise a first, second, third, and fourth polypeptide chain, wherein (a) the first polypeptide chain comprises a domain A, a domain B, a domain D, and a domain E, wherein the domains are arranged, from N-terminus to C-terminus, in a A-B-D-E orientation, and domain A has a VL amino acid sequence, domain B has a CH3 amino acid sequence, domain D has a CH2 amino acid sequence, and domain E has a constant region domain amino acid sequence; (b) the second polypeptide chain comprises a domain F and a domain G, wherein the domains are arranged, from N-terminus to C-terminus, in a F-G orientation, and wherein domain F has a VH amino acid sequence and domain G has a CH3 amino acid sequence; (c) the third polypeptide chain comprises a domain H, a domain I, a domain J, and a domain K, wherein the domains are arranged, from N-terminus to C-terminus, in a H-I-J-K orientation, and wherein domain H has a variable region domain amino acid sequence, domain I has a constant region domain amino acid sequence, domain J has a CH2 amino acid sequence, and K has a constant region domain amino acid sequence; (d) the fourth polypeptide chain comprises a domain L and a domain M, wherein the domains are arranged, from N-terminus to C-terminus, in a L-M orientation, and wherein domain L has a variable region domain amino acid sequence and domain M has a constant region domain amino acid sequence; (e) the first and the second polypeptides are associated through an interaction between the A and the F domains and an interaction between the B and the G domains; (f) the third and the fourth polypeptides are associated through an interaction between the H and the L domains and an interaction between the I and the M domains; and (g) the first and the third polypeptides are associated through an interaction between the D and the J domains and an interaction between the E and the K domains to form the bivalent B-body architecture.

In a preferred embodiment, domain E has a CH3 amino acid sequence, domain H has a VL amino acid sequence, domain I has a CL amino acid sequence, domain K has a CH3 amino acid sequence, domain L has a VH amino acid sequence, and domain M has a CH1 amino acid sequence.

In certain embodiments, the interaction between the A domain and the F domain form a first antigen binding site specific for a first antigen, and the interaction between the H domain and the L domain form a second antigen binding site specific for a second antigen, and the bivalent B-body architecture is a bispecific bivalent B-body architecture. In certain embodiments, the interaction between the A domain and the F domain form a first antigen binding site specific for a first antigen, and the interaction between the H domain and the L domain form a second antigen binding site specific for the first antigen, and the bivalent B-body architecture is a monospecific bivalent B-body architecture.

### 6.4.1. Bivalent Bispecific B-Body “BC1”

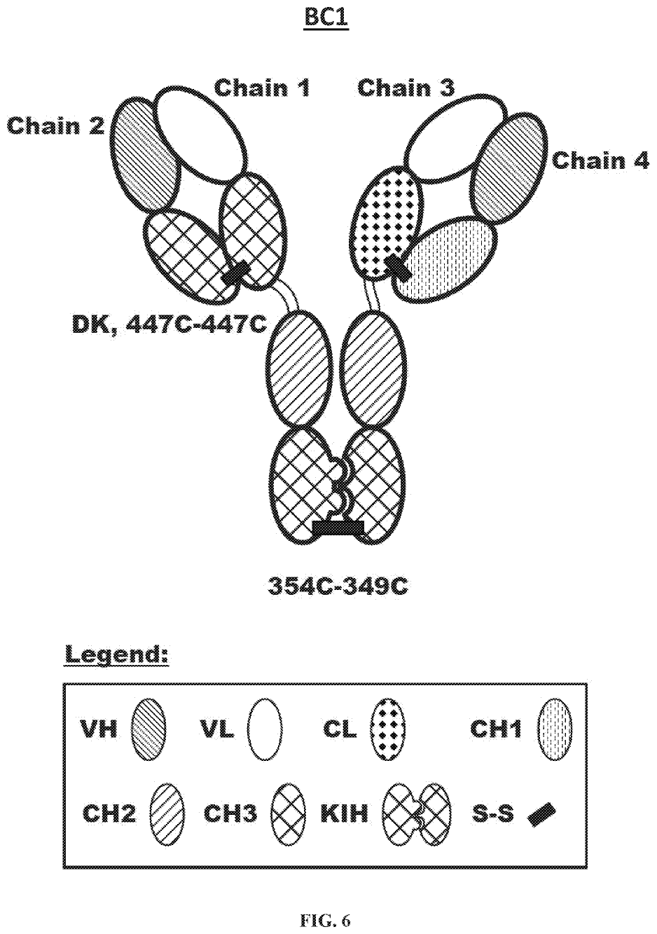

With reference to FIG. 3 and FIG. 6, in a series of embodiments, trivalent trispecific binding molecules are provided that are based on the bivalent B-body architecture having a first, second, third, and fourth polypeptide chain, wherein (a) the first polypeptide chain comprises a domain A, a domain B, a domain D, and a domain E, wherein the domains are arranged, from N-terminus to C-terminus, in a A-B-D-E orientation, and domain A has a first VL amino acid sequence, domain B has a human IgG1 CH3 amino acid sequence with a T366K mutation and a C-terminal extension incorporating a KSC tripeptide sequence that is followed by the DKTHT motif of an IgG1 hinge region, domain D has a human IgG1 CH2 amino acid sequence, and domain E has human IgG1 CH3 amino acid with a S354C and T366W mutation; (b) the second polypeptide chain has a domain F and a domain G, wherein the domains are arranged, from N-terminus to C-terminus, in a F-G orientation, and wherein domain F has a first VH amino acid sequence and domain G has a human IgG1 CH3 amino acid sequence with a L351D mutation and a C-terminal extension incorporating a GEC amino acid disulfide motif; (c) the third polypeptide chain has a domain H, a domain I, a domain J, and a domain K, wherein the domains are arranged, from N-terminus to C-terminus, in a H-I-J-K orientation, and wherein domain H has a second VL amino acid sequence, domain I has a human CL kappa amino acid sequence, domain J has a human IgG1 CH2 amino acid sequence, and K has a human IgG1 CH3 amino acid sequence with a Y349C, a D356E, a L358M, a T366S, a L368A, and a Y407V mutation; (d) the fourth polypeptide chain has a domain L and a domain M, wherein the domains are arranged, from N-terminus to C-terminus, in a L-M orientation, and wherein domain L has a second VH amino acid sequence and domain M has a human IgG1 CH1 amino acid sequence; (e) the first and the second polypeptides are associated through an interaction between the A and the F domains and an interaction between the B and the G domains; (f) the third and the fourth polypeptides are associated through an interaction between the H and the L domains and an interaction between the I and the M domains; (g) the first and the third polypeptides are associated through an interaction between the D and the J domains and an interaction between the E and the K domains to form the bivalent B-body architecture; (h) domain A and domain F form a first antigen binding site specific for a first antigen; and (i) domain H and domain L form a second antigen binding site specific for a second antigen.

In preferred embodiments, the first polypeptide chain has the sequence SEQ ID NO:8, the second polypeptide chain has the sequence SEQ ID NO:9, the third polypeptide chain has the sequence SEQ ID NO:10, and the fourth polypeptide chain has the sequence SEQ ID NO:11.

### 6.4.2. Bivalent Bispecific B-Body “BC6”

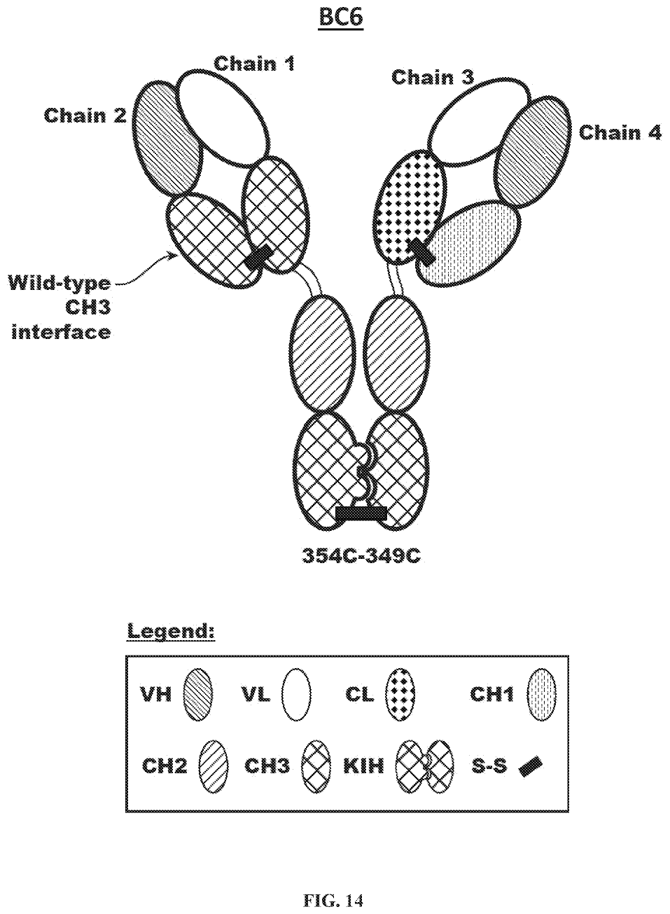

With reference to FIG. 3 and FIG. 14, in a series of embodiments, trivalent trispecific binding molecules are provided that are based on the bivalent B-body architecture having a first, second, third, and fourth polypeptide chain, wherein (a) the first polypeptide chain comprises a domain A, a domain B, a domain D, and a domain E, wherein the domains are arranged, from N-terminus to C-terminus, in a A-B-D-E orientation, and domain A has a first VL amino acid sequence, domain B has a human IgG1 CH3 amino acid sequence with a C-terminal extension incorporating a KSC tripeptide sequence that is followed by the DKTHT motif of an IgG1 hinge region, domain D has a human IgG1 CH2 amino acid sequence, and domain E has human IgG1 CH3 amino acid with a S354C and a T366W mutation; (b) the second polypeptide chain has a domain F and a domain G, wherein the domains are arranged, from N-terminus to C-terminus, in a F-G orientation, and wherein domain F has a first VH amino acid sequence and domain G has a human IgG1 CH3 amino acid sequence with a C-terminal extension incorporating a GEC amino acid disulfide motif; (c) the third polypeptide chain has a domain H, a domain I, a domain J, and a domain K, wherein the domains are arranged, from N-terminus to C-terminus, in a H-I-J-K orientation, and wherein domain H has a second VL amino acid sequence, domain I has a human CL kappa amino acid sequence, domain J has a human IgG1 CH2 amino acid sequence, and K has a human IgG1 CH3 amino acid sequence with a Y349C, a D356E, a L358M, a T366S, a L368A, and a Y407V mutation; (d) the fourth polypeptide chain has a domain L and a domain M, wherein the domains are arranged, from N-terminus to C-terminus, in a L-M orientation, and wherein domain L has a second VH amino acid sequence and domain M has a human IgG1 amino acid sequence; (e) the first and the second polypeptides are associated through an interaction between the A and the F domains and an interaction between the B and the G domains; (f) the third and the fourth polypeptides are associated through an interaction between the H and the L domains and an interaction between the I and the M domains; (g) the first and the third polypeptides are associated through an interaction between the D and the J domains and an interaction between the E and the K domains to form the bivalent B-body architecture; (h) domain A and domain F form a first antigen binding site specific for a first antigen; and (i) domain H and domain L form a second antigen binding site specific for a second antigen.

### 6.4.3. Bivalent Bispecific B-Body “BC28”

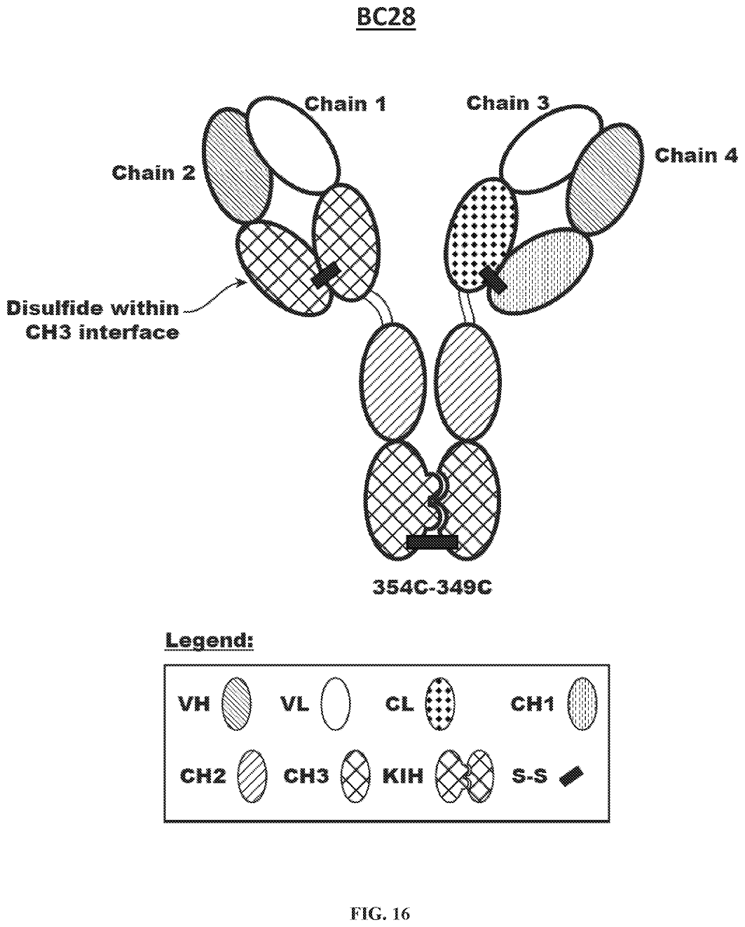

With reference to FIG. 3 and FIG. 16, in a series of embodiments, trivalent trispecific binding molecules are provided that are based on the bivalent B-body architecture having a first, second, third, and fourth polypeptide chain, wherein (a) the first polypeptide chain comprises a domain A, a domain B, a domain D, and a domain E, wherein the domains are arranged, from N-terminus to C-terminus, in a A-B-D-E orientation, and domain A has a first VL amino acid sequence, domain B has a human IgG1 CH3 amino acid sequence with a Y349C mutation and a C-terminal extension incorporating a PGK tripeptide sequence that is followed by the DKTHT motif of an IgG1 hinge region, domain D has a human IgG1 CH2 amino acid sequence, and domain E has a human IgG1 CH3 amino acid with a S354C and a T366W mutation; (b) the second polypeptide chain has a domain F and a domain G, wherein the domains are arranged, from N-terminus to C-terminus, in a F-G orientation, and wherein domain F has a first VH amino acid sequence and domain G has a human IgG1 CH3 amino acid sequence with a S354C mutation and a C-terminal extension incorporating a PGK tripeptide sequence; (c) the third polypeptide chain has a domain H, a domain I, a domain J, and a domain K, wherein the domains are arranged, from N-terminus to C-terminus, in a H-I-J-K orientation, and wherein domain H has a second VL amino acid sequence, domain I has a human CL kappa amino acid sequence, domain J has a human IgG1 CH2 amino acid sequence, and K has a human IgG1 CH3 amino acid sequence with a Y349C, a D356E, a L358M, a T366S, a L368A, and a Y407V; (d) the fourth polypeptide chain has a domain L and a domain M, wherein the domains are arranged, from N-terminus to C-terminus, in a L-M orientation, and wherein domain L has a second VH amino acid sequence and domain M has a human IgG1 CH1 amino acid sequence; (e) the first and the second polypeptides are associated through an interaction between the A and the F domains and an interaction between the B and the G domains; (f) the third and the fourth polypeptides are associated through an interaction between the H and the L domains and an interaction between the I and the M domains; (g) the first and the third polypeptides are associated through an interaction between the D and the J domains and an interaction between the E and the K domains to form the bivalent B-body architecture; (h) domain A and domain F form a first antigen binding site specific for a first antigen; and (i) domain H and domain L form a second antigen binding site specific for a second antigen.

In preferred embodiments, the first polypeptide chain has the sequence SEQ ID NO:24, the second polypeptide chain has the sequence SEQ ID NO:25, the third polypeptide chain has the sequence SEQ ID NO:10, and the fourth polypeptide chain has the sequence SEQ ID NO:11.

### 6.4.4. Bivalent Bispecific B-Body “BC44”

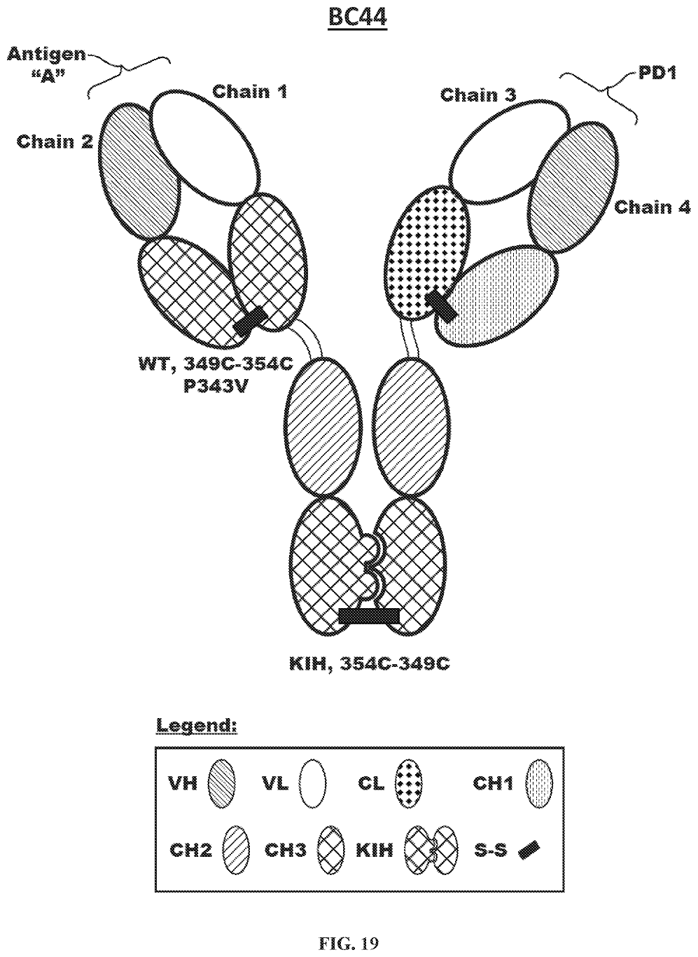

With reference to FIG. 3 and FIG. 19, in a series of embodiments, trivalent trispecific binding molecules are provided that are based on the bivalent B-body architecture having a first, second, third, and fourth polypeptide chain, wherein (a) the first polypeptide chain comprises a domain A, a domain B, a domain D, and a domain E, wherein the domains are arranged, from N-terminus to C-terminus, in a A-B-D-E orientation, and domain A has a first VL amino acid sequence, domain B has a human IgG1 CH3 amino acid sequence with a Y349C mutation, a P343V mutation, and a C-terminal extension incorporating a PGK tripeptide sequence that is followed by the DKTHT motif of an IgG1 hinge region, domain D has a human IgG1 CH2 amino acid sequence, and domain E has human IgG1 CH3 amino acid with a S354C mutation and a T366W mutation; (b) the second polypeptide chain has a domain F and a domain G, wherein the domains are arranged, from N-terminus to C-terminus, in a F-G orientation, and wherein domain F has a first VH amino acid sequence and domain G has a human IgG1 CH3 amino acid sequence with a S354C mutation and a C-terminal extension incorporating a PGK tripeptide sequence; (c) the third polypeptide chain has a domain H, a domain I, a domain J, and a domain K, wherein the domains are arranged, from N-terminus to C-terminus, in a H-I-J-K orientation, and wherein domain H has a second VL amino acid sequence, domain I has a human CL kappa amino acid sequence, domain J has a human IgG1 CH2 amino acid sequence, and K has a human IgG1 CH3 amino acid sequence with a Y349C, T366S, L368A, and aY407V; (d) the fourth polypeptide chain has a domain L and a domain M, wherein the domains are arranged, from N-terminus to C-terminus, in a L-M orientation, and wherein domain L has a second VH amino acid sequence and domain M has a human IgG1 amino acid sequence; (e) the first and the second polypeptides are associated through an interaction between the A and the F domains and an interaction between the B and the G domains; (f) the third and the fourth polypeptides are associated through an interaction between the H and the L domains and an interaction between the I and the M domains; and (g) the first and the third polypeptides are associated through an interaction between the D and the J domains and an interaction between the E and the K domains to form the bivalent B-body architecture; (h) domain A and domain F form a first antigen binding site specific for a first antigen; and (i) domain H and domain L form a second antigen binding site specific for a second antigen.

In preferred embodiments, the first polypeptide chain has the sequence SEQ ID NO:32, the second polypeptide chain has the sequence SEQ ID NO:25, the third polypeptide chain has the sequence SEQ ID NO:10, and the fourth polypeptide chain has the sequence SEQ ID NO:11.

### 6.4.5. Bivalent Binding Molecules with IgA-CH3 Domain Pairs

With reference to FIG. 3, in a series of embodiments, the binding molecule has a first, second, third, and fourth polypeptide chain, wherein (a) the first polypeptide chain comprises a domain A, a domain B, a domain D, and a domain E, wherein the domains are arranged, from N-terminus to C-terminus, in a A-B-D-E orientation, and domain A has a variable region amino acid sequence, domain B has a human IgA CH3 amino acid sequence, domain D has a human IgG1 CH2 amino acid sequence, and domain E has human IgG1 CH3 amino acid sequence; (b) the second polypeptide chain has a domain F and a domain G, wherein the domains are arranged, from N-terminus to C-terminus, in a F-G orientation, and wherein domain F has a variable region amino acid sequence and domain G has a human IgA CH3 amino acid sequence; (c) the third polypeptide chain has a domain H, a domain I, a domain J, and a domain K, wherein the domains are arranged, from N-terminus to C-terminus, in a H-I-J-K orientation, and wherein domain H has a variable region amino acid sequence, domain I has a constant region amino acid sequence, domain J has a human IgG1 CH2 amino acid sequence, and domain K has a human IgG1 CH3 amino acid sequence; (d) the fourth polypeptide chain has a domain L and a domain M, wherein the domains are arranged, from N-terminus to C-terminus, in a L-M orientation, and wherein domain L has a variable region amino acid sequence and domain M has a constant region amino acid sequence; (e) the first and the second polypeptides are associated through an interaction between the A and the F domains and an interaction between the B and the G domains; (f) the third and the fourth polypeptides are associated through an interaction between the H and the L domains and an interaction between the I and the M domains; (g) the first and the third polypeptides are associated through an interaction between the D and the J domains and an interaction between the E and the K domains to form the binding molecule. In some embodiments, domain A and domain F form a first antigen binding site specific for a first antigen; and domain H and domain L form a second antigen binding site specific for a second antigen.

In some embodiments, domain A comprises a VH amino acid sequence, domain F comprises a VL amino acid sequence, domain H comprises a VH amino acid sequence, domain I comprises a CH1 amino acid sequence, domain L comprises a VL amino acid sequence, and domain M comprises a CL amino acid sequence. In some embodiments, domain A comprises a first VH amino acid sequence and domain F comprises a first VL amino acid sequence, domain H comprises a second VH amino acid sequence and domain L comprises a second VL amino acid sequence.

In preferred embodiments, domain A comprises a VL amino acid sequence, domain F comprises a VH amino acid sequence, domain H comprises a VL amino acid sequence, domain L comprises a VH amino acid sequence, domain I comprises a CL amino acid sequence, and domain M comprises a CH1 amino acid sequence. In some embodiments, the CL amino acid sequence is a CL-kappa sequence. In some embodiments, domain A comprises a first VL amino acid sequence and domain F comprises a first VH amino acid sequence, domain H comprises a second VL amino acid sequence and domain L comprises a second VH amino acid sequence.

In some embodiments, domain E further comprises a S354C and T366W mutation in the human IgG1 CH3 amino acid sequence. In some embodiments, domain K further comprises a Y349C, a D356E, a L358M, a T366S, a L368A, and a Y407V mutation in the human IgG1 CH3 amino acid sequence.

In some embodiments, domain B comprises a first CH3 linker sequence as described in Section 6.3.20.3 that is followed by the DKTHT motif of an IgG1 hinge region; and domain G comprises a second CH3 linker sequence as described in Section 6.3.20.3. In some embodiments, the first CH3 linker sequence associates with the second CH3 linker sequence by formation of a disulfide bridge between cysteine residues of the first and second CH3 linker sequences.

In some embodiments, the first CH3 linker and the second CH3 linker are identical. In some embodiments, the first CH3 linker and second CH3 linker are non-identical. In some embodiments, the first CH3 linker and second CH3 linker differ in length by 1-6 amino acids. In some embodiments, the first CH3 linker and second CH3 linker differ in length by 1-3 amino acids. In some embodiments, the first CH3 linker is AGC and the second CH3 linker is AGKGSC. In some embodiments, the first CH3 linker is AGKGC and the second CH3 linker is AGC. In some embodiments, the first CH3 linker is AGKGSC and the second CH3 linker is AGC. In some embodiments, the first CH3 linker is AGKC and the second CH3 linker is AGC.

In some embodiments, the binding molecule further comprises one or more CH1/CL modifications as described in Sections 6.3.10.3 and 6.3.10.3.

In some embodiments, the binding molecule further comprises a modification that reduces effector function as described in Section 6.8.4.

### 6.5. Specific Trivalent Binding Molecules

### 6.5.1. Trivalent 1×2 Bispecific B-Body “BC28-1×2”

With reference to Section 6.4.3. and FIG. 26, in a series of embodiments, the trivalent trispecific binding molecules based on the bivalent B-body architectures described above comprise a sixth polypeptide chain, wherein (a) the third polypeptide chain further comprises a domain R and a domain S, wherein the domains are arranged, from N-terminus to C-terminus, in a R-S-H-I-J-K orientation, and wherein domain R has the first VL amino acid sequence and domain S has a human IgG1 CH3 amino acid sequence with a Y349C mutation and a C-terminal extension incorporating a PGK tripeptide sequence that is followed by GSGSGS linker peptide connecting domain S to domain H; (b) the trivalent trispecific binding molecule further comprises a sixth polypeptide chain, comprising: a domain T and a domain U, wherein the domains are arranged, from N-terminus to C-terminus, in a T-U orientation, and wherein domain T has the first VH amino acid sequence and domain U has a human IgG1 CH3 amino acid sequence with a S354C mutation and a C-terminal extension incorporating a PGK tripeptide sequence; (c) the third and the sixth polypeptides are associated through an interaction between the R and the T domains and an interaction between the S and the U domains to form the trivalent trispecific binding molecule, and (d) domain R and domain T form a third antigen binding site specific for the first antigen.

In preferred embodiments, the first polypeptide chain has the sequence SEQ ID NO:24, the second polypeptide chain has the sequence SEQ ID NO:25, the third polypeptide chain has the sequence SEQ ID NO:37, the fourth polypeptide chain has the sequence SEQ ID NO:11, and the sixth polypeptide chain has the sequence SEQ ID NO:25.

### 6.5.2. Trivalent 1×2 Trispecific B-Body “BC28-1×1×1a”

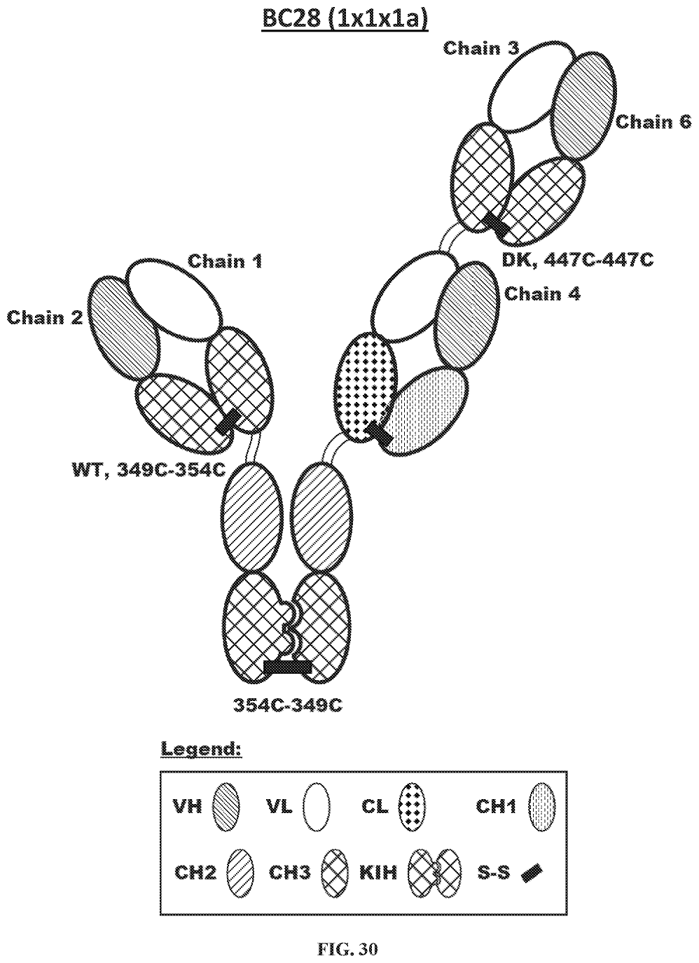

With reference to Section 6.4.3. and FIG. 26 and FIG. 30, in a series of embodiments, the trivalent trispecific binding molecules based on the bivalent B-body architectures described above further comprise a sixth polypeptide chain, wherein (a) the third polypeptide chain further comprises a domain R and a domain S, wherein the domains are arranged, from N-terminus to C-terminus, in a R-S-H-I-J-K orientation, and wherein domain R has a third VL amino acid sequence and domain S has a human IgG1 CH3 amino acid sequence with a T366K mutation and a C-terminal extension incorporating a KSC tripeptide sequence that is followed by GSGSGS linker peptide connecting domain S to domain H; (b) the trivalent trispecific binding molecule further comprises a sixth polypeptide chain, comprising: a domain T and a domain U, wherein the domains are arranged, from N-terminus to C-terminus, in a T-U orientation, and wherein domain T has a third VH amino acid sequence and domain U has a human IgG1 CH3 amino acid sequence with a L351D mutation and a C-terminal extension incorporating a GEC amino acid disulfide motif; and (c) the third and the sixth polypeptides are associated through an interaction between the R and the T domains and an interaction between the S and the U domains to form the trivalent trispecific binding molecule, and (d) domain R and domain T form a third antigen binding site specific for a third antigen.

In preferred embodiments, the first polypeptide chain has the sequence SEQ ID NO:24, the second polypeptide chain has the sequence SEQ ID NO:25, the third polypeptide chain has the sequence SEQ ID NO:45, the fourth polypeptide chain has the sequence SEQ ID NO:11, and the sixth polypeptide chain has the sequence SEQ ID NO: 53.

### 6.6. Other Binding Molecule Platforms

The various antibody platforms described above are not limiting. The trivalent trispecific binding molecules described herein, including specific CDR subsets, can be based on any compatible binding molecule platform including, but not limited to, full-length antibodies, Fab fragments, Fvs, scFvs, tandem scFvs, Diabodies, scDiabodies, DARTs, tandAbs, minibodies, camelid VHH, and other antibody fragments or formats known to those skilled in the art. Exemplary antibody and antibody fragment formats are described in detail in Brinkmann et al. (MABS, 2017, Vol. 9, No. 2, 182-212), herein incorporated by reference for all that it teaches.

In some embodiments, the trivalent trispecific binding molecule is based on a CrossMab™ platform. CrossMab™ antibodies are described in U.S. Pat. Nos. 8,242,247; 9,266,967; and 8,227,577, U.S. Patent Application Pub. No. 20120237506, U.S. Patent Application Pub. No. US20090162359, WO2016016299, WO2015052230. In some embodiments, the trivalent trispecific binding molecule is based on a bivalent, bispecific antibody, comprising: a) the light chain and heavy chain of an antibody specifically binding to a first antigen; and b) the light chain and heavy chain of an antibody specifically binding to a second antigen, wherein constant domains CL and CH1 from the antibody specifically binding to a second antigen are replaced by each other. In some embodiments, the trivalent trispecific binding molecule is based on the format structured with reference to Section 6.4 and FIG. 3, wherein A is VH, B is CH1, D is CH2, E is CH3, F is VL, G is CL, H is VL or VH, I is CL, J is CH2, K is CH3, L is VH or VL, and M is CH1.

In some embodiments, the trivalent trispecific binding molecule is based on an antibody having a general architecture described in U.S. Pat. No. 8,871,912 and WO2016087650. In some embodiments, the trivalent trispecific binding molecule is based on a domain-exchanged antibody comprising a light chain (LC) composed of VL-CH3, and a heavy chain (HC) comprising VH-CH3-CH2-CH3, wherein the VL-CH3 of the LC dimerizes with the VH-CH3 of the HC thereby forming a domain-exchanged LC/HC dimer comprising a CH3LC/CH3HC domain pair. In some embodiments, the trivalent trispecific binding molecule is based on the format structured with reference to Section 6.4 and FIG. 3, wherein A is VH, B is CH3, D is CH2, E is CH3, F is VL, G is CH3, H is VH, I is CH1, J is CH2, K is CH3, L is VL, and M is CL.

In some embodiments, the trivalent trispecific binding molecule is based on the platform as described in WO2017011342. In some embodiments, the trivalent trispecific binding molecule is based on the format structured with reference to Section 6.4 and FIG. 3, wherein A is VH or VL, B is CH2 from IgM or IgE, D is CH2, E is CH3, F is VL or VH, G is CH2 from IgM or IgE, H is VH, I is CH1, J is CH2, K is CH3, L is VL, and M is CL.

In some embodiments, the trivalent trispecific binding molecule is based on the platform as described in WO2006093794. In some embodiments, the trivalent trispecific binding molecule is based on the format structured with reference to Section 6.4 and FIG. 3, wherein A is VH, B is CH1, D is CH2, E is CH3, F is VL, G is CL, H is VL, I is CL or CH1, J is CH2, K is CH3, L is VH, and M is CH1 or CL.

### 6.7. Antigen Specificities

Antigen binding sites potentially relevant to the binding molecules described herein may be chosen to specifically bind a wide variety of molecular targets. For example, an antigen binding site or sites may specifically bind E-Cad, CLDN7, FGFR2b, N-Cad, Cad-11, FGFR2c, ERBB2, ERBB3, FGFR1, FOLR1, IGF-Ira, GLP1R, PDGFRa, PDGFRb, EPHB6, ABCG2, CXCR4, CXCR7, Integrin-avb3, SPARC, VCAM, ICAM, Annexin, TNFα, CD137, angiopoietin 2, angiopoietin 3, BAFF, beta amyloid, C5, CA-125, CD147, CD125, CD147, CD152, CD19, CD20, CD22, CD23, CD24, CD25, CD274, CD28, CD3, CD30, CD33, CD37, CD4, CD40, CD44, CD44v4, CD44v6, CD44v7, CD50, CD51, CD52, CEA, CSF1R, CTLA-2, DLL4, EGFR, EPCAM, HER3, GD2 ganglioside, GDF-8, Her2/neu, CD2221, IL-17A, IL-12, IL-23, IL-13, IL-6, IL-23, an integrin, CD11a, MUC1, Notch, TAG-72, TGFβ, TRAIL-R2, VEGF-A, VEGFR-1, VEGFR2, VEGFc, hematopoietins (four-helix bundles) (such as EPO (erythropoietin), IL-2 (T-cell growth factor), IL-3 (multicolony CSF), IL-4 (BCGF-1, BSF-1), IL-5 (BCGF-2), IL-6 IL-4 (IFN-β2, BSF-2, BCDF), IL-7, IL-8, IL-9, IL-11, IL-13 (P600), G-CSF, IL-15 (T-cell growth factor), GM-CSF (granulocyte macrophage colony stimulating factor), OSM (OM, oncostatin M), and LIF (leukemia inhibitory factor)); interferons (such as IFN-γ, IFN-α, and IFN-β); immunoglobin superfamily (such as B7.1 (CD80), and B7.2 (B70, CD86)); TNF family (such as TNF-α (cachectin), TNF-β (lymphotoxin, LT, LT-α), LT-β, Fas, CD27, CD30, and 4-1BBL); and those unassigned to a particular family (such as TGF-β, IL 1α, IL-113, IL-1 RA, IL-10 (cytokine synthesis inhibitor F), IL-12 (NK cell stimulatory factor), MIF, IL-16, IL-17 (mCTLA-8), and/or IL-18 (IGIF, interferon-γ inducing factor)); in embodiments relating to bispecific antibodies, the antibody may for example bind two of these targets. Furthermore, the Fc portion of the heavy chain of an antibody may be used to target Fc receptor-expressing cells such as the use of the Fc portion of an IgE antibody to target mast cells and basophils. An antigen binding site or sites may be chosen that specifically binds the TNF family of receptors including, but not limited to, TNFR1 (also known as CD120a and TNFRSF1A), TNFR2 (also known as CD120b and TNFRSF1B), TNFRSF3 (also known as LTβR), TNFRSF4 (also known as OX40 and CD134), TNFRSF5 (also known as CD40), TNFRSF6 (also known as FAS and CD95), TNFRSF6B (also known as DCR3), TNFRSF7 (also known as CD27), TNFRSF8 (also known as CD30), TNFRSF9 (also known as 4-1BB), TNFRSF10A (also known as TRAILR1, DR4, and CD26), TNFRSF10B (also known as TRAILR2, DR5, and CD262), TNFRSF10C (also known as TRAILR3, DCR1, CD263), TNFRSF10D (also known as TRAILR4, DCR2, and CD264), TNFRSF11A (also known as RANK and CD265), TNFRSF11B (also known as OPG), TNFRSF12A (also known as FN14, TWEAKR, and CD266), TNFRSF13B (also known as TACI and CD267), TNFRSF13C (also known as BAFFR, BR3, and CD268), TNFRSF14 (also known as HVEM and CD270), TNFRSF16 (also known as NGFR, p75NTR, and CD271), or TNFRSF17 (also known as BCMA and CD269), TNFRSF18 (also known as GITR and CD357), TNFRSF19 (also known as TROY, TAJ, and TRADE), TNFRSF21 (also known as CD358), TNFRSF25 (also known as Apo-3, TRAMP, LARD, or WS-1), EDA2R (also known as XEDAR).

An antigen binding site or sites may be chosen that specifically binds immune-oncology targets including, but not limited to, checkpoint inhibitor targets such as PD1, PDL1, CTLA-4, PDL2, B7-H3, B7-H4, BTLA, TIM3, GALS, LAG3, VISTA, KIR, 2B4, BY55, and CGEN-15049.

In particular embodiments, the trivalent trispecific binding molecule has antigen binding sites that specifically bind two tumor associated antigens and a T cell surface expressed molecule. In a specific embodiment, the trivalent trispecific binding molecule has antigen binding sites that specifically bind two tumor associated antigens and the T cell surface expressed protein CD3. Without wishing to be bound by theory, the trivalent trispecific binding molecule that specifically binds the two tumor antigens and the T cell surface expressed molecule (i.e., CD3) can direct T cell mediated killing (cytotoxicity) of cells expressing the two tumor associated antigens through redirecting T cells to the tumor associated antigens expressing cells (i.e., target cells). T cell mediated killing using bispecific anti-CD3 molecules is described in detail in U.S. Pub. No. 2006/0193852, herein incorporated by reference in its entirety. In some embodiments, the T cell surface expressed molecule is selected from any molecule capable of redirecting T cells to a target cell. In some embodiments, the one or more affinities of individual ABSs for the two tumor associated antigens do not have has a K_{D} value that qualifies as specifically binding their respective antigens or epitopes on their own, but the avidity of the trivalent trispecific binding molecule for a specific target cell expressing the two tumor associated antigens has a K_{D} value such that the interaction is a specific binding interaction.

In a series of embodiments, an antigen binding site or sites may be chosen that specifically target tumor-associated cells. In various embodiments, the antigen binding site or sites specifically target tumor associated immune cells. In certain embodiments, the antigen binding site or sites specifically target tumor associated regulatory T cells (Tregs). In specific embodiments, a binding molecule has antigen binding sites specific for antigens selected from one or more of CD25, OX40, CTLA-4, and NRP1 such that the binding molecule specifically targets tumor associated regulatory T cells. In specific embodiments, a binding molecule has antigen binding sites that specifically bind CD25 and OX40, CD25 and CTLA-4, CD25 and NRP1, OX40 and CTLA-4, OX40 and NRP1, or CTLA-4 and NRP1 such that the binding molecule specifically targets tumor associated regulatory T cells. In preferred embodiments, a bispecific bivalent binding molecule has antigen binding sites that specifically bind CD25 and OX40, CD25 and CTLA-4, CD25 and NRP1, OX40 and CTLA-4, OX40 and NRP1, or CTLA-4 and NRP1 such that the binding molecule specifically targets tumor associated regulatory T cells. In specific embodiments, the specific targeting of the tumor associated regulatory T cells results in depletion (e.g. killing) of the regulatory T cells. In preferred embodiments, the depletion of the regulatory T cells is mediated by an antibody-drug conjugate (ADC) modification, such as an antibody conjugated to a toxin, as discussed in more detail below in Section 6.8.1.

### 6.8. Further Modifications

In a further series of embodiments, the trivalent trispecific binding molecule has additional modifications.

### 6.8.1. Antibody-Drug Conjugates

In various embodiments, the trivalent trispecific binding molecule is conjugated to a therapeutic agent (i.e. drug) to form a trivalent trispecific binding molecule-drug conjugate. Therapeutic agents include, but are not limited to, chemotherapeutic agents, imaging agents (e.g. radioisotopes), immune modulators (e.g. cytokines, chemokines, or checkpoint inhibitors), and toxins (e.g. cytotoxic agents). In certain embodiments, the therapeutic agents are attached to the trivalent trispecific binding molecule through a linker peptide, as discussed in more detail below in Section 6.8.3.

Methods of preparing antibody-drug conjugates (ADCs) that can be adapted to conjugate drugs to the trivalent trispecific binding molecules disclosed herein are described, e.g., in U.S. Pat. No. 8,624,003 (pot method), U.S. Pat. No. 8,163,888 (one-step), U.S. Pat. No. 5,208,020 (two-step method), U.S. Pat. Nos. 8,337,856, 5,773,001, 7,829,531, 5,208,020, 7,745,394, WO 2017/136623, WO 2017/015502, WO 2017/015496, WO 2017/015495, WO 2004/010957, WO 2005/077090, WO 2005/082023, WO 2006/065533, WO 2007/030642, WO 2007/103288, WO 2013/173337, WO 2015/057699, WO 2015/095755, WO 2015/123679, WO 2015/157286, WO 2017/165851, WO 2009/073445, WO 2010/068759, WO 2010/138719, WO 2012/171020, WO 2014/008375, WO 2014/093394, WO 2014/093640, WO 2014/160360, WO 2015/054659, WO 2015/195925, WO 2017/160754, Storz (MAbs. 2015 November-December; 7(6): 989-1009), Lambert et al. (Adv Ther, 2017 34: 1015), Diamantis et al. (British Journal of Cancer, 2016, 114, 362-367), Carrico et al. (Nat Chem Biol, 2007. 3: 321-2), We et al. (Proc Natl Acad Sci USA, 2009. 106: 3000-5), Rabuka et al. (Curr Opin Chem Biol., 2011 14: 790-6), Hudak et al. (Angew Chem Int Ed Engl., 2012: 4161-5), Rabuka et al. (Nat Protoc., 2012 7:1052-67), Agarwal et al. (Proc Natl Acad Sci USA., 2013, 110: 46-51), Agarwal et al. (Bioconjugate Chem., 2013, 24: 846-851), Barfield et al. (Drug Dev. and D., 2014, 14:34-41), Drake et al. (Bioconjugate Chem., 2014, 25:1331-41), Liang et al. (J Am Chem Soc., 2014, 136:10850-3), Drake et al. (Curr Opin Chem Biol., 2015, 28:174-80), and York et al. (BMC Biotechnology, 2016, 16(1):23), each of which is hereby incorporated by reference in its entirety for all that it teaches.

### 6.8.2. Additional Binding Moieties

In various embodiments, the trivalent trispecific binding molecule has modifications that comprise one or more additional binding moieties. In certain embodiments the binding moieties are antibody fragments or antibody formats including, but not limited to, full-length antibodies, Fab fragments, Fvs, scFvs, tandem scFvs, Diabodies, scDiabodies, DARTs, tandAbs, minibodies, camelid VHH, and other antibody fragments or formats known to those skilled in the art. Exemplary antibody and antibody fragment formats are described in detail in Brinkmann et al. (MABS, 2017, Vol. 9, No. 2, 182-212), herein incorporated by reference for all that it teaches.

In particular embodiments, the one or more additional binding moieties are attached to the C-terminus of the first or third polypeptide chain. In particular embodiments, the one or more additional binding moieties are attached to the C-terminus of both the first and third polypeptide chain. In particular embodiments, the one or more additional binding moieties are attached to the C-terminus of both the first and third polypeptide chains. In certain embodiments, individual portions of the one or more additional binding moieties are separately attached to the C-terminus of the first and third polypeptide chains such that the portions form the functional binding moiety.

In particular embodiments, the one or more additional binding moieties are attached to the N-terminus of any of the polypeptide chains (e.g. the first, second, third, fourth, fifth, or sixth polypeptide chains). In certain embodiments, individual portions of the additional binding moieties are separately attached to the N-terminus of different polypeptide chains such that the portions form the functional binding moiety.

In certain embodiments, the one or more additional binding moieties are specific for a different antigen or epitope of the ABSs within the trivalent trispecific binding molecule. In certain embodiments, the one or more additional binding moieties are specific for the same antigen or epitope of the ABSs within the trivalent trispecific binding molecule. In certain embodiments, wherein the modification is two or more additional binding moieties, the additional binding moieties are specific for the same antigen or epitope. In certain embodiments, wherein the modification is two or more additional binding moieties, the additional binding moieties are specific for different antigens or epitopes.

In certain embodiments, the one or more additional binding moieties are attached to the trivalent trispecific binding molecule using in vitro methods including, but not limited to, reactive chemistry and affinity tagging systems, as discussed in more detail below in Section 6.8.3. In certain embodiments, the one or more additional binding moieties are attached to the trivalent trispecific binding molecule through Fc-mediated binding (e.g. Protein A/G). In certain embodiments, the one or more additional binding moieties are attached to the trivalent trispecific binding molecule using recombinant DNA techniques, such as encoding the nucleotide sequence of the fusion product between the trivalent trispecific binding molecule and the additional binding moieties on the same expression vector (e.g. plasmid).

### 6.8.3. Functional/Reactive Groups

In various embodiments, the trivalent trispecific binding molecule has modifications that comprise functional groups or chemically reactive groups that can be used in downstream processes, such as linking to additional moieties (e.g. drug conjugates and additional binding moieties, as discussed in more detail above in Sections 6.8.1. and 6.8.2.) and downstream purification processes.

In certain embodiments, the modifications are chemically reactive groups including, but not limited to, reactive thiols (e.g. maleimide based reactive groups), reactive amines (e.g. N-hydroxysuccinimide based reactive groups), “click chemistry” groups (e.g. reactive alkyne groups), and aldehydes bearing formylglycine (FGly). In certain embodiments, the modifications are functional groups including, but not limited to, affinity peptide sequences (e.g. HA, HIS, FLAG, GST, MBP, and Strep systems etc.). In certain embodiments, the functional groups or chemically reactive groups have a cleavable peptide sequence. In particular embodiments, the cleavable peptide is cleaved by means including, but not limited to, photocleavage, chemical cleavage, protease cleavage, reducing conditions, and pH conditions. In particular embodiments, protease cleavage is carried out by intracellular proteases. In particular embodiments, protease cleavage is carried out by extracellular or membrane associated proteases. ADC therapies adopting protease cleavage are described in more detail in Choi et al. (Theranostics, 2012; 2(2): 156-178.), the entirety of which is hereby incorporated by reference for all it teaches.

### 6.8.4. Reduced Effector Function

In certain embodiments, the trivalent trispecific binding molecule has one or more engineered mutations in an amino acid sequence of an antibody domain that reduce the effector functions generally associated with antibody binding. Effector functions include, but are not limited to, cellular functions that result from an Fc receptor binding to an Fc portion of an antibody, such as antibody dependent cellular cytotoxicity (ADCC), complement fixation (e.g. C1q binding), antibody dependent cellular-mediated phagocytosis (ADCP), opsonization. Engineered mutations that reduce the effector functions are described in more detail in U.S. Pub. No. 2017/0137530, Armour, et al. (Eur. J. Immunol. 29(8) (1999) 2613-2624), Shields, et al. (J. Biol. Chem. 276(9) (2001) 6591-6604), and Oganesyan, et al. (Acta Cristallographica D64 (2008) 700-704), each herein incorporated by reference in their entirety.

In specific embodiments, the trivalent trispecific binding molecule has one or more engineered mutations in an amino acid sequence of an antibody domain that reduce binding of an Fc portion of the trivalent trispecific binding molecule by FcR receptors. In some embodiments, the FcR receptors are FcRγ receptors. In particular embodiments, the FcR receptors are FcγRIIa and/or FcγRIIIA receptors.

In specific embodiments, the one or more engineered mutations that reduce effector function are mutations in a CH2 domain of an antibody. In various embodiments, the one or more engineered mutations are at position L234 and L235 of the CH2 domain. In particular embodiments, the one or more engineered mutations are L234A and L235A of the CH2 domain. In other embodiments, the one or more engineered mutations are at position L234, L235, and P329 of the CH2 domain. In particular embodiments, the one or more engineered mutations are L234A, L235A, and P329G of the CH2 domain. In preferred embodiments, the one or more engineered mutations are L234A, L235A, and P329K of the CH2 domain.

### 6.9. Methods of Purification

A method of purifying a trivalent trispecific binding molecule comprising a B-body platform is provided herein.

In a series of embodiments, the method comprises the steps of: i) contacting a sample comprising the trivalent trispecific binding molecule with a CH1 binding reagent, wherein the trivalent trispecific binding molecule comprises at least a first, a second, a third, and a fourth polypeptide chain associated in a complex, wherein the complex comprises at least one CH1 domain, or portion thereof, and wherein the number of CH1 domains in the complex is at least one fewer than the valency of the complex, and wherein the contacting is performed under conditions sufficient for the CH1 binding reagent to bind the CH1 domain, or portion thereof; and ii) purifying the complex from one or more incomplete complexes, wherein the incomplete complexes do not comprise the first, the second, the third, and the fourth polypeptide chain.

In a typical, naturally occurring, antibody, two heavy chains are associated, each of which has a CH1 domain as the second domain, numbering from N-terminus to C-terminus. Thus, a typical antibody has two CH1 domains. CH1 domains are described in more detail in Section 6.3.10.1. In a variety of the trivalent trispecific binding molecules described herein, the CH1 domain typically found in the protein has been substituted with another domain, such that the number of CH1 domains in the protein is effectively reduced. In a non-limiting illustrative example, the CH1 domain of a typical antibody can be substituted with a CH3 domain, generating an antigen-binding protein having only a single CH1 domain.

Trivalent trispecific binding molecules can also refer to molecules based on antibody architectures that have been engineered such that they no longer possess a typical antibody architecture. For example, an antibody can be extended at its N or C terminus to increase the valency (described in more detail in Section 6.3.15.1) of the antigen-binding protein, and in certain instances the number of CH1 domains is also increased beyond the typical two CH1 domains. Such molecules can also have one or more of their CH1 domains substituted, such that the number of CH1 domains in the protein is at least one fewer than the valency of the antigen-binding protein. In some embodiments, the number of CH1 domains that are substituted by other domains generates a trivalent trispecific binding molecule having only a single CH1 domain. In other embodiments, the number of CH1 domains substituted by another domain generates a trivalent trispecific binding molecule having two or more CH1 domains, but at least one fewer than the valency of the antigen-binding protein. In particular embodiments, where a trivalent trispecific binding molecule has two or more CH1 domains, the multiple CH1 domains can all be in the same polypeptide chain. In other particular embodiments, where a trivalent trispecific binding molecule has two or more CH1 domains, the multiple CH1 domains can be a single CH1 domain in multiple copies of the same polypeptide chain present in the complete complex.

### 6.9.1. CH1 Binding Reagents

In exemplary non-limiting methods of purifying trispecific trivalent binding molecules, a sample comprising the trivalent trispecific binding molecules is contacted with CH1 binding reagents. CH1 binding reagents, as described herein, can be any molecule that specifically binds a CH1 epitope. The various CH1 sequences that provide the CH1 epitope are described in more detail in Section 6.3.10.1, and specific binding is described in more detail in Section 6.3.15.1.

In some embodiments, CH1 binding reagents are derived from immunoglobulin proteins and have an antigen binding site (ABS) that specifically binds the CH1 epitope. In particular embodiments, the CH1 binding reagent is an antibody, also referred to as an “anti-CH1 antibody.” The anti-CH1 antibody can be derived from a variety of species. In particular embodiments, the anti-CH1 antibody is a mammalian antibody, including, but not limited to mouse, rat, hamster, rabbit, camel, donkey, goat, and human antibodies. In specific embodiments, the anti-CH1 antibody is a single-domain antibody. Single-domain antibodies, as described herein, have a single variable domain that forms the ABS and specifically binds the CH1 epitope. Exemplary single-domain antibodies include, but are not limited to, heavy chain antibodies derived from camels and sharks, as described in more detail in international application WO 2009/011572, herein incorporated by reference for all it teaches. In a preferred embodiment, the anti-CH1 antibody is a camel derived antibody (also referred to as a “camelid antibody”). Exemplary camelid antibodies include, but are not limited to, human IgG-CH1 CaptureSelect™ (ThermoFisher, #194320010) and human IgA-CH1 (ThermoFisher, #194311010). In some embodiments, the anti-CH1 antibody is a monoclonal antibody. Monoclonal antibodies are typically produced from cultured antibody-producing cell lines. In other embodiments, the anti-CH1 antibody is a polyclonal antibody, i.e., a collection of different anti-CH1 antibodies that each recognize the CH1 epitope. Polyclonal antibodies are typically produced by collecting the antibody containing serum of an animal immunized with the antigen of interest, or fragment thereof, here CH1.

In some embodiments, CH1 binding reagents are molecules not derived from immunoglobulin proteins. Examples of such molecules include, but are not limited to, aptamers, peptoids, and affibodies, as described in more detail in Perret and Boschetti (Biochimie, February 2018, Vol 145:98-112).

### 6.9.2. Solid Supports

In exemplary non-limiting methods of purifying trispecific trivalent binding molecules, the CH1 binding reagent can be attached to a solid support in various embodiments of the invention. Solid supports, as described herein, refers to a material to which other entities can be attached or immobilized, e.g., the CH1 binding reagent. Solid supports, also referred to as “carriers,” are described in more detail in international application WO 2009/011572.

In specific embodiments, the solid support comprises a bead or nanoparticle. Examples of beads and nanoparticles include, but are not limited to, agarose beads, polystyrene beads, magnetic nanoparticles (e.g., Dynabeads™, ThermoFisher), polymers (e.g., dextran), synthetic polymers (e.g., Sepharose™), or any other material suitable for attaching the CH1 binding reagent. In particular embodiments, the solid support is modified to enable attachment of the CH1 binding reagent. Example of solid support modifications include, but are not limited to, chemical modifications that form covalent bonds with proteins (e.g., activated aldehyde groups) and modifications that specifically pair with a cognate modification of a CH1 binding reagent (e.g., biotin-streptavidin pairs, disulfide linkages, polyhistidine-nickel, or “click-chemistry” modifications such as azido-alkynyl pairs).

In certain embodiments, the CH1 binding reagent is attached to the solid support prior to the CH1 binding reagent contacting the trivalent trispecific binding molecules, herein also referred to as an “anti-CH1 resin.” In some embodiments, anti-CH1 resins are dispersed in a solution. In other embodiments, anti-CH1 resins are “packed” into a column. The anti-CH1 resin is then contacted with the trivalent trispecific binding molecules and the CH1 binding reagents specifically bind the trivalent trispecific binding molecules.

In other embodiments, the CH1 binding reagent is attached to the solid support after the CH1 binding reagent contacts the trivalent trispecific binding molecules. As a non-limiting illustration, a CH1 binding reagent with a biotin modification can be contacted with the trivalent trispecific binding molecules, and subsequently the CH1 binding reagent/trivalent trispeicifc binding molecule mixture can be contacted with streptavidin modified solid support to attach the CH1 binding reagent to the solid support, including CH1 binding reagents specifically bound to the trivalent trispecific binding molecules.

In methods wherein the CH1 binding reagents are attached to solid supports, in a variety of embodiments, the bound trispecific trivalent binding molecules are released, or “eluted,” from the solid support forming an eluate having the trivalent trispecific binding molecules. In some embodiments, the bound trispecific trivalent binding molecules are released through reversing the paired modifications (e.g., reduction of the disulfide linkage), adding a reagent to compete off the trivalent trispecific binding molecules (e.g., adding imidazole that competes with a polyhistidine for binding to nickel), cleaving off the trivalent trispecific binding molecules (e.g., a cleavable moiety can be included in the modification), or otherwise interfering with the specific binding of the CH1 binding reagent for the trivalent trispecific binding molecule. Methods that interfere with specific binding include, but are not limited to, contacting trispecific trivalent binding molecules bound to CH1 binding reagents with a low-pH solution. In preferred embodiment, the low-pH solution comprises 0.1 M acetic acid pH 4.0. In other embodiments, the bound trispecific trivalent binding molecules can be contacted with a range of low-pH solutions, i.e., a “gradient.”

### 6.9.3. Further Purification

In some embodiments of the exemplary non-limiting methods, a single iteration of the method using the steps of contacting the trivalent trispecific binding molecules with the CH1 binding reagents, followed by eluting the trivalent trispecific binding molecules, is used to purify the trivalent trispecific binding molecules from the one or more incomplete complexes. In particular embodiments, no other purifying step is performed. In other embodiments, one or more additional purification steps are performed to further purify the trivalent trispecific binding molecules from the one or more incomplete complexes. The one or more additional purification steps include, but are not limited to, purifying the trivalent trispecific binding molecules based on other protein characteristics, such as size (e.g., size exclusion chromatography), charge (e.g., ion exchange chromatography), or hydrophobicity (e.g., hydrophobicity interaction chromatography). In a preferred embodiment, an additional cation exchange chromatograph is performed. Additionally, the trivalent trispecific binding molecules can be further purified repeating contacting the trivalent trispecific binding molecules with the CH1 binding reagents as described above, as well as modifying the CH1 purification method between iterations, e.g., using a step elution for the first iteration and a gradient elution for a subsequent elution.

### 6.9.4. Assembly and Purity of Complexes

In the embodiments of the present invention, at least four distinct polypeptide chains associate together to form a complete complex, i.e., the trivalent trispecific binding molecule. However, incomplete complexes can also form that do not contain the at least four distinct polypeptide chains. For example, incomplete complexes may form that only have one, two, or three of the polypeptide chains. In other examples, an incomplete complex may contain more than three polypeptide chains, but does not contain the at least four distinct polypeptide chains, e.g., the incomplete complex inappropriately associates with more than one copy of a distinct polypeptide chain. The method of the invention purifies the complex, i.e., the completely assembled trispecific trivalent binding molecule, from incomplete complexes.

Methods to assess the efficacy and efficiency of the purification steps are well known to those skilled in the art and include, but are not limited to, SDS-PAGE analysis, ion exchange chromatography, size exclusion chromatography, and mass spectrometry. Purity can also be assessed according to a variety of criteria. Examples of criterion include, but are not limited to: 1) assessing the percentage of the total protein in an eluate that is provided by the completely assembled trispecific trivalent binding molecule, 2) assessing the fold enrichment or percent increase of the method for purifying the desired products, e.g., comparing the total protein provided by the completely assembled trispecific trivalent binding molecule in the eluate to that in a starting sample, 3) assessing the percentage of the total protein or the percent decrease of undesired products, e.g., the incomplete complexes described above, including determining the percent or the percent decrease of specific undesired products (e.g., unassociated single polypeptide chains, dimers of any combination of the polypeptide chains, or trimers of any combination of the polypeptide chains). Purity can be assessed after any combination of methods described herein. For example, purity can be assessed after a single iteration of using the anti-CH1 binding reagent, as described herein, or after additional purification steps, as described in more detail in Section 6.9.3. The efficacy and efficiency of the purification steps may also be used to compare the methods described using the anti-CH1 binding reagent to other purification methods known to those skilled in the art, such as Protein A purification.

### 6.10. Methods of Manufacturing

The trivalent trispecific binding molecules described herein can readily be manufactured by expression using standard cell free translation, transient transfection, and stable transfection approaches currently used for antibody manufacture. In specific embodiments, Expi293 cells (ThermoFisher) can be used for production of the trivalent trispecific binding molecules using protocols and reagents from ThermoFisher, such as ExpiFectamine, or other reagents known to those skilled in the art, such as polyethylenimine as described in detail in Fang et al. (Biological Procedures Online, 2017, 19:11), herein incorporated by reference for all it teaches.

As further described in the Examples below, the expressed proteins can be readily separated from undesired proteins and protein complexes using a CH1 affinity resin, such as the CaptureSelect CH1 resin and provided protocol from ThermoFisher. Other purification strategies include, but are not limited to, use of Protein A, Protein G, or Protein A/G reagents. Further purification can be affected using ion exchange chromatography as is routinely used in the art.

### 6.11. Pharmaceutical Compositions

In another aspect, pharmaceutical compositions are provided that comprise a trivalent trispecific binding molecule as described herein and a pharmaceutically acceptable carrier or diluent. In typical embodiments, the pharmaceutical composition is sterile.

In various embodiments, the pharmaceutical composition comprises the trivalent trispecific binding molecule at a concentration of 0.1 mg/ml-100 mg/ml. In specific embodiments, the pharmaceutical composition comprises the trivalent trispecific binding molecule at a concentration of 0.5 mg/ml, 1 mg/ml, 1.5 mg/ml, 2 mg/ml, 2.5 mg/ml, 5 mg/ml, 7.5 mg/ml, or 10 mg/ml. In some embodiments, the pharmaceutical composition comprises the trivalent trispecific binding molecule at a concentration of more than 10 mg/ml. In certain embodiments, the trivalent trispecific binding molecule is present at a concentration of 20 mg/ml, 25 mg/ml, 30 mg/ml, 35 mg/ml, 40 mg/ml, 45 mg/ml, or even 50 mg/ml or higher. In particular embodiments, the trivalent trispecific binding molecule is present at a concentration of more than 50 mg/ml.

In various embodiments, the pharmaceutical compositions are described in more detail in U.S. Pat. Nos. 8,961,964, 8,945,865, 8,420,081, 6,685,940, 6,171,586, 8,821,865, 9,216,219, U.S. application Ser. No. 10/813,483, WO 2014/066468, WO 2011/104381, and WO 2016/180941, each of which is incorporated herein in its entirety.

### 6.12. Methods of Treatment

In another aspect, methods of treatment are provided, the methods comprising administering a trivalent trispecific binding molecule as described herein to a patient in an amount effective to treat the patient.

In some embodiments, an antibody of the present disclosure may be used to treat a cancer. The cancer may be a cancer from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In some embodiments, the cancer may be a neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

An antibody of the present disclosure may be administered to a subject per se or in the form of a pharmaceutical composition for the treatment of, e.g., cancer, autoimmunity, transplantation rejection, post-traumatic immune responses, graft-versus-host disease, ischemia, stroke, and infectious diseases, for example by targeting viral antigens, such as gp120 of HIV.

### 6.13. Examples

The following examples are provided by way of illustration, not limitation.

### 6.13.1. Methods

Non-limiting, illustrative methods for the purification of the various antigen-binding proteins and their use in various assays are described in more detail below.

### 6.13.1.1. Expi293 Expression

The various antigen-binding proteins tested were expressed using the Expi293 transient transfection system according to manufacturer's instructions. Briefly, four plasmids coding for four individual chains were mixed at 1:1:1:1 mass ratio, unless otherwise stated, and transfected with ExpiFectamine 293 transfection kit to Expi 293 cells. Cells were cultured at 37° C. with 8% CO2, 100% humidity and shaking at 125 rpm. Transfected cells were fed once after 16-18 hours of transfections. The cells were harvested at day 5 by centrifugation at 2000 g for 10 munities. The supernatant was collected for affinity chromatography purification.

### 6.13.1.2. Protein A and Anti-CH1 Purification

Cleared supernatants containing the various antigen-binding proteins were separated using either a Protein A (ProtA) resin or an anti-CH1 resin on an AKTA Purifier FPLC. In examples where a head-to-head comparison was performed, supernatants containing the various antigen-binding proteins were split into two equal samples. For ProtA purification, a 1 mL Protein A column (GE Healthcare) was equilibrated with PBS (5 mM sodium potassium phosphate pH 7.4, 150 mM sodium chloride). The sample was loaded onto the column at 5 ml/min. The sample was eluted using 0.1 M acetic acid pH 4.0. The elution was monitored by absorbance at 280 nm and the elution peaks were pooled for analysis. For anti-CH1 purification, a 1 mL CaptureSelect™ XL column (ThermoFisher) was equilibrated with PBS. The sample was loaded onto the column at 5 ml/min. The sample was eluted using 0.1 M acetic acid pH 4.0. The elution was monitored by absorbance at 280 nm and the elution peaks were pooled for analysis.

### 6.13.1.3. SDS-Page Analysis

Samples containing the various separated antigen-binding proteins were analyzed by reducing and non-reducing SDS-PAGE for the presence of complete product, incomplete product, and overall purity. 2 μg of each sample was added to 15 μL SDS loading buffer. Reducing samples were incubated in the presence of 10 mM reducing agent at 75° C. for 10 minutes. Non-reducing samples were incubated at 95° C. for 5 minutes without reducing agent. The reducing and non-reducing samples were loaded into a 4-15% gradient TGX gel (BioRad) with running buffer and run for 30 minutes at 250 volts. Upon completion of the run, the gel was washed with DI water and stained using GelCode Blue Safe Protein Stain (ThermoFisher). The gels were destained with DI water prior to analysis. Densitometry analysis of scanned images of the destained gels was performed using standard image analysis software to calculate the relative abundance of bands in each sample.

### 6.13.1.4. IEX Chromatography

Samples containing the various separated antigen-binding proteins were analyzed by cation exchange chromatography for the ratio of complete product to incomplete product and impurities. Cleared supernatants were analyzed with a 5-ml MonoS (GE Lifesciences) on an AKTA Purifier FPLC. The MonoS column was equilibrated with buffer A 10 mM MES pH 6.0. The samples were loaded onto the column at 2 ml/min. The sample was eluted using a 0-30% gradient with buffer B (10 mM MES pH 6.0, 1 M sodium chloride) over 6 CV. The elution was monitored by absorbance at 280 nm and the purity of the samples were calculated by peak integration to identify the abundance of the monomer peak and contaminants peaks. The monomer peak and contaminant peaks were separately pooled for analysis by SDS-PAGE as described above.

### 6.13.1.5. Analytical SEC Chromatography

Samples containing the various separated antigen-binding proteins were analyzed by analytical size exclusion chromatography for the ratio of monomer to high molecular weight product and impurities. Cleared supernatants were analyzed with an industry standard TSK G3000SW×1 column (Tosoh Bioscience) on an Agilent 1100 HPLC. The TSK column was equilibrated with PBS. 25 μL of each sample at 1 mg/mL was loaded onto the column at 1 ml/min. The sample was eluted using an isocratic flow of PBS for 1.5 CV. The elution was monitored by absorbance at 280 nm and the elution peaks were analyzed by peak integration.

### 6.13.1.6. Mass Spec

Samples containing the various separated antigen-binding proteins were analyzed by mass spectrometry to confirm the correct species by molecular weight. All analysis was performed by a third-party research organization. Briefly, samples were treated with a cocktail of enzymes to remove glycosylation. Samples were both tested in the reduced format to specifically identify each chain by molecular weight. Samples were all tested under non-reducing conditions to identify the molecular weights of all complexes in the samples. Mass spec analysis was used to identify the number of unique products based on molecular weight.

### 6.13.1.7. Antibody Discovery by Phage Display

Phage display of human Fab libraries is carried out using standard protocols. Biotinylated antigen of interest are purchased or synthesized. Phage clones are screened for the ability to bind antigens of interest by phage ELISA using standard protocols. Briefly, Fab-formatted phage libraries are constructed using expression vectors capable of replication and expression in phage (also referred to as a phagemid). Both the heavy chain and the light chain were encoded for in the same expression vector, where the heavy chain was fused to a truncated variant of the phage coat protein pIII. The light chain and heavy chain-pIII fusion are expressed as separate polypeptides and assemble in the bacterial periplasm, where the redox potential enables disulfide bond formation, to form the phage display antibody containing the candidate ABS. The phage display heavy chain (SEQ ID NO:74) and light chain (SEQ ID NO:75) scaffolds used in the library are listed below, where a lower case “x” represents CDR amino acids that were varied to create the library, and bold italic represents the CDR sequences that were constant.

Specific libraries are generated by introducing diversity into VL and VH CDR sequences. Diversity is created through Kunkel mutagenesis using primers to introduce diversity into VL CDR3 and VH CDR1 (H1), CDR2 (H2) and CDR3 (H3) to mimic the diversity found in the natural antibody repertoire, as described in more detail in Kunkel, T A (PNAS Jan. 1, 1985. 82 (2) 488-492), herein incorporated by reference in its entirety. Briefly, single-stranded DNA are prepared from isolated phage using standard procedures and Kunkel mutagenesis carried out. Chemically synthesized DNA are then electroporated into TG1 cells, followed by recovery. Recovered cells are sub-cultured and infected with M13K07 helper phage to produce the phage library.

Phage panning is performed using standard procedures. Briefly, the first round of phage panning are performed with target immobilized on streptavidin magnetic beads which are subjected to ˜5×10¹² phages from the prepared library in a volume of 1 mL in PBST-2% BSA. After a one-hour incubation, the bead-bound phage are separated from the supernatant using a magnetic stand. Beads are washed three times to remove non-specifically bound phage and are then added to ER2738 cells (5 mL) at OD₆₀₀˜0.6. After 20 minutes, infected cells are sub-cultured in 25 mL 2×YT+Ampicillin and M13K07 helper phage and allowed to grow overnight at 37° C. with vigorous shaking. The next day, phage are prepared using standard procedures by PEG precipitation. Pre-clearance of phage specific to SAV-coated beads is performed prior to panning. The second round of panning is performed using the KingFisher magnetic bead handler with 100 nM bead-immobilized antigen using standard procedures. In total, 3-4 rounds of phage panning are performed to enrich in phage displaying Fabs specific for the target antigen. Target-specific enrichment is confirmed using polyclonal and monoclonal phage ELISA. DNA sequencing is used to determine isolated Fab clones containing a candidate ABS.

To measure binding affinity in antigen binder discovery campaigns, the VL and VH domains identified in the phage screen described above were formatted into a bivalent monospecific native human full-length IgG1 architecture and immobilized to a biosensor on an Octet (Pall ForteBio) biolayer interferometer. Soluble antigens of interest are then added to the system and binding measured.

For experiments performed using the B-Body format, VL variable regions of individual clones are formatted into Domain A and/or H, and VH region into Domain F and/or L of a bivalent 1×1 B-Body “BC1” scaffold shown below and with reference to FIG. 3.

“BC1” Scaffold:
1^{st} polypeptide chain (SEQ ID NO:78)
   Domain A=Antigen 1 B-Body Domain A/H Scaffold (SEQ ID NO:76)
   Domain B=CH3 (T366K; 445K, 446S, 447C tripeptide insertion)
   Domain D=CH2
   Domain E=CH3 (T366W, S354C)
2^{nd} polypeptide chain (SEQ ID NO:79):
   Domain F=Antigen 1 B-Body Domain F/L Scaffold (SEQ ID NO:77)
   Domain G=CH3 (L351D; 445G, 446E, 447C tripeptide insertion)
3^{rd} polypeptide chain (SEQ ID NO:80):
   Domain H=Antigen 2 B-Body Domain A/H Scaffold (SEQ ID NO:76)
   Domain I=CL (Kappa)
   Domain J=CH2
   Domain K=CH3 (Y349C, D356E, L358M, T366S, L368A, Y407V)
4^{th} polypeptide chain (SEQ ID NO:81):
   Domain L=Antigen 2 B-Body Domain F/L Scaffold (SEQ ID NO:77)
   Domain M=CH1.

For BC1 1×2 formats, the variable domains are formatted into Chains 1, 2, and 4 above, as well as the Chain 3 scaffold with the sequence of SEQ ID NO:82, where the junction between domain S and domain H is a 10 amino acid linker having the sequence TASSGGSSSG (SEQ ID NO:83). Polypeptide Chain 2 and Chain 6 are identical in the 1×2 format.

### 6.13.1.8. NFκB GFP Jurkat T Cell Stimulation Assay

The NFκB/Jurkat/GFP transcriptional reporter cell line was purchased from System Biosciences (Cat #TR850-1). The anti-CD28 antibody used for co-stimulation was purchased from BD Pharmingen (Cat 555725). The Solution C background suppression dye was purchased from Life Technologies (K1037). Briefly, the Jurkat cells (effector cells, E) were mixed with the tumor cells (T) at an E:T ratio of 2:1 to 4:1 in the presence of a dilution series of B-body™ antibodies and an anti-CD28 antibody at 1 ug/mL in a 96 well black walled clear bottom plate. The plate was incubated at 37° C./5% CO2 for 6 hours, following which a 6× solution of Solution C background suppressor was added to the plate and GFP fluorescence was read out on a plate reader. EC50 values, referring to the concentration of antibody that gives the half-maximal response, were determined from the dilution series.

### 6.13.1.9. Primary T Cell Cytotoxicity Assay

Cells expressing the target tumor antigen (T) and effector cells (E) were mixed at an E:T ratio ranging from 3:1 to 10:1. Effector cells used include PBMCs or isolated cytotoxic CD8+ T cells. The candidate redirecting T cell antibody was added in a dilution series to the cells. Controls included media only controls, tumor cell only controls, and untreated E:T cell controls. The mixed cells and control conditions were incubated at 37° C./5% CO2 for 40-50 hours. The Cytotoxicity Detection Kit Plus (LDH) was purchased from Sigma (Cat 4744934001) and the manufacturer's directions were followed. Briefly, lysis solution added to tumor cells served as the 100% cytotoxicity control and untreated E:T cells served as the 0% cytotoxicity control. The level of lactate dehydrogenase (LDH) in each sample was determined via absorbance at 490 nm and normalize to the 100% and 0% controls. EC50 values, referring to the concentration of antibody that gives the half-maximal response, were determined from the dilution series.

### 6.13.2. Example 1: Bivalent Monospecific Construct and Bivalent Bispecific Construct

A bivalent monospecific B-Body recognizing TNFα was constructed with the following architecture (VL(Certolizumab)-CH3(Knob)-CH2-CH3/VH(Certolizumab)-CH3(Hole)) using standard molecular biology procedures. In this construct,
1^{st} polypeptide chain (SEQ ID NO:1)
   Domain A=VL (certolizumab)
   Domain B=CH3 (IgG1) (knob: S354C+T366W)
   Domain D=CH2 (IgG1)
   Domain E=CH3 (IgG1)
2^{nd} polypeptide chain (SEQ ID NO:2)
   Domain F=VH (certolizumab)
   Domain G=CH3 (IgG1) (hole:Y349C, T366S, L368A, Y407V)
3^{rd} polypeptide chain:
   identical to the 1^{st} polypeptide chain
4^{th} polypeptide chain:
   identical to the 2^{nd} polypeptide chain.

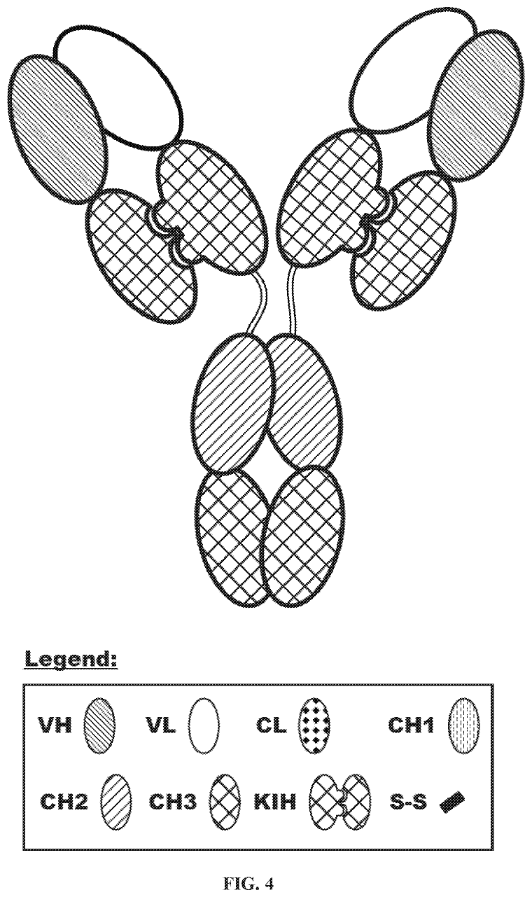

Domain and polypeptide chain references are in accordance with FIG. 3. The overall construct architecture is illustrated in FIG. 4. The sequence of the first polypeptide chain, with domain A identified in shorthand as “(VL)”, is provided in SEQ ID NO:1. The sequence of the second polypeptide chain, with domain F identified in shorthand as “(VH)”, is provided in SEQ ID NO:2.

The full-length construct was expressed in an E. coli cell free protein synthesis expression system for ˜18 hours at 26° C. with gentle agitation. Following expression, the cell-free extract was centrifuged to pellet insoluble material and the supernatant was diluted 2× with 10× Kinetic Buffer (Forte Bio) and used as the analyte for biolayer interferometry.

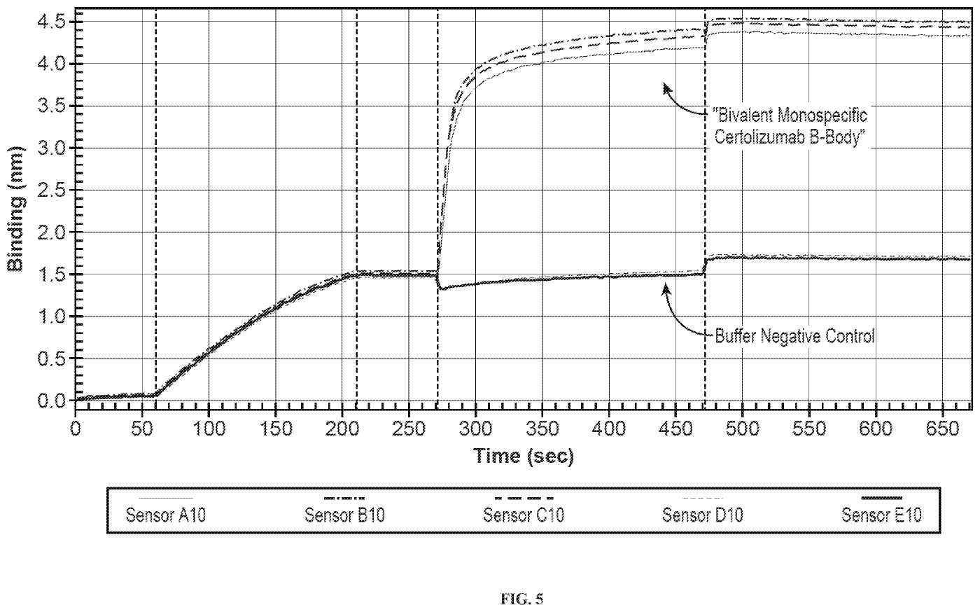

Biotinylated TNFα was immobilized on a streptavidin sensor to give a wave shift response of ˜1.5 nm. After establishing a baseline with 10× kinetic buffer, the sensor was dipped into the antibody construct analyte solution. The construct gave a response of ˜3 nm, comparable to the traditional IgG format of certolizumab, demonstrating the ability of the bivalent monospecific construct to assemble into a functional, full-length antibody. Results are shown in FIG. 5.

We also constructed a bivalent bispecific antibody with the following domain architecture:
1^{st} polypeptide chain: VL-CH3-CH2-CH3(Knob)
2nd polypeptide chain: VH-CH3
3^{rd} polypeptide chain: VL-CL-CH2-CH3(Hole)
4^{th} polypeptide chain VH-CH1.

The sequences (except for the variable region sequences) are provided respectively in SEQ ID NO:3 (1st polypeptide chain), SEQ ID NO:4 (2nd polypeptide chain), SEQ ID NO:5 (3rd polypeptide chain), SEQ ID NO:6 (4th polypeptide chain).

### 6.13.3. Example 2: Bivalent Bispecific B-Body “BC1”

We constructed a bivalent bispecific construct, termed “BC1”, specific for PD1 and a second antigen, “Antigen A”). Salient features of the “BC1” architecture are illustrated in FIG. 6.

In greater detail, with domain and polypeptide chain references in accordance with FIG. 3 and modifications from native sequence indicated in parentheses, the architecture was:
1^{st} polypeptide chain (SEQ ID NO:8)
   Domain A=VL (“Antigen A”)
   Domain B=CH3 (T366K; 445K, 446S, 447C tripeptide insertion)
   Domain D=CH2
   Domain E=CH3 (T366W, S354C)
2^{nd} polypeptide chain (SEQ ID NO:9):
   Domain F=VH (“Antigen A”)
   Domain G=CH3 (L351D; 445G, 446E, 447C tripeptide insertion)
3^{rd} polypeptide chain (SEQ ID NO:10):
   Domain H=VL (“Nivo”)
   Domain I=CL (Kappa)
   Domain J=CH2
   Domain K=CH3 (Y349C, D356E, L358M, T366S, L368A, Y407V)
4^{th} polypeptide chain (SEQ ID NO:11):
   Domain L=VH (“Nivo”)
   Domain M=CH1.

The A domain (SEQ ID NO: 12) and F domain (SEQ ID NO: 16) form an antigen binding site (A:F) specific for “Antigen A”. The H domain has the VH sequence from nivolumab and the L domain has the VL sequence from nivolumab; H and L associate to form an antigen binding site (H:L) specific for human PD1.

The B domain (SEQ ID NO:13) has the sequence of human IgG1 CH3 with several mutations: T366K, 445K, 446S, and 447C insertion. The T366K mutation is a charge pair cognate of the L351D residue in Domain G. The “447C” residue on domain B comes from the C-terminal KSC tripeptide insertion.

Domain D (SEQ ID NO: 14) has the sequence of human IgG1 CH2

Domain E (SEQ ID NO: 15) has the sequence of human IgG1 CH3 with the mutations T366W and S354C. The 366W is the “knob” mutation. The 354C introduces a cysteine that is able to form a disulfide bond with the cognate 349C mutation in Domain K.

Domain G (SEQ ID NO:17) has the sequence of human IgG1 CH3 with the following mutations: L351D, and 445G, 446E, 447C tripeptide insertion. The L351D mutation introduces a charge pair cognate to the Domain B T366K mutation. The “447C” residue on domain G comes from the C-terminal GEC tripeptide insertion.

Domain I (SEQ ID NO: 19) has the sequence of human C kappa light chain (Cκ)

Domain J [SEQ ID NO: 20] has the sequence of human IgG1 CH2 domain, and is identical to the sequence of domain D.

Domain K [SEQ ID NO: 21] has the sequence of human IgG1 CH3 with the following changes: Y349C, D356E, L358M, T366S, L368A, Y407V. The 349C mutation introduces a cysteine that is able to form a disulfide bond with the cognate 354C mutation in Domain E. The 356E and L358M introduce isoallotype amino acids that reduce immunogenicity. The 366S, 368A, and 407V are “hole” mutations.

Domain M [SEQ ID NO: 23] has the sequence of the human IgG1 CH1 region.

“BC1” could readily be expressed at high levels using mammalian expression at concentrations greater than 100 μg/ml.

We found that the bivalent bispecific “BC1” protein could easily be purified in a single step using a CH1-specific CaptureSelect™ affinity resin from ThermoFisher.

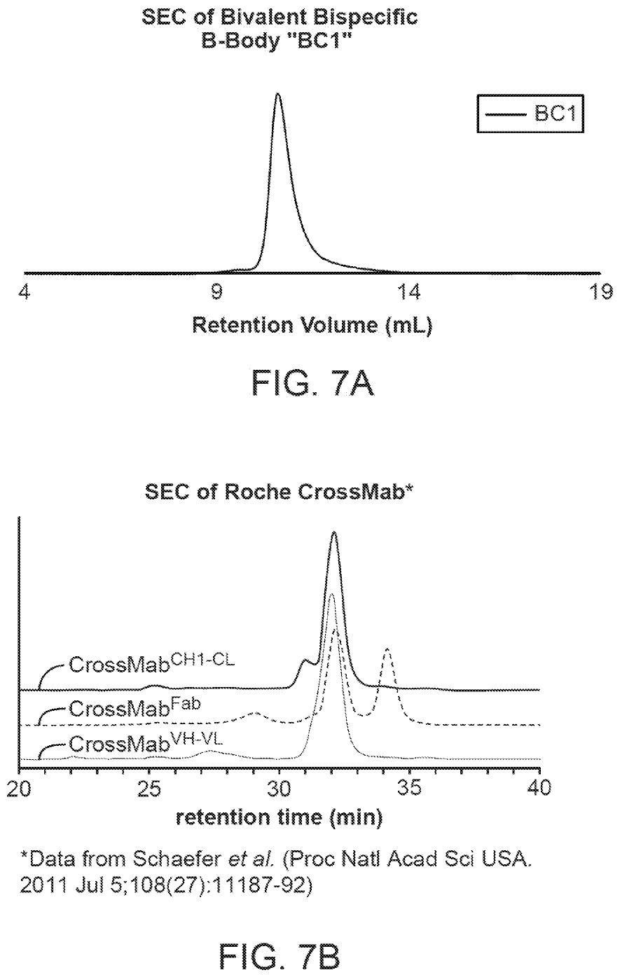

As shown in FIG. 7A, SEC analysis demonstrates that a single-step CH1 affinity purification step yields a single, monodisperse peak via gel filtration in which >98% is monomer. FIG. 7B shows comparative literature data of SEC analysis of a CrossMab bivalent antibody construct.

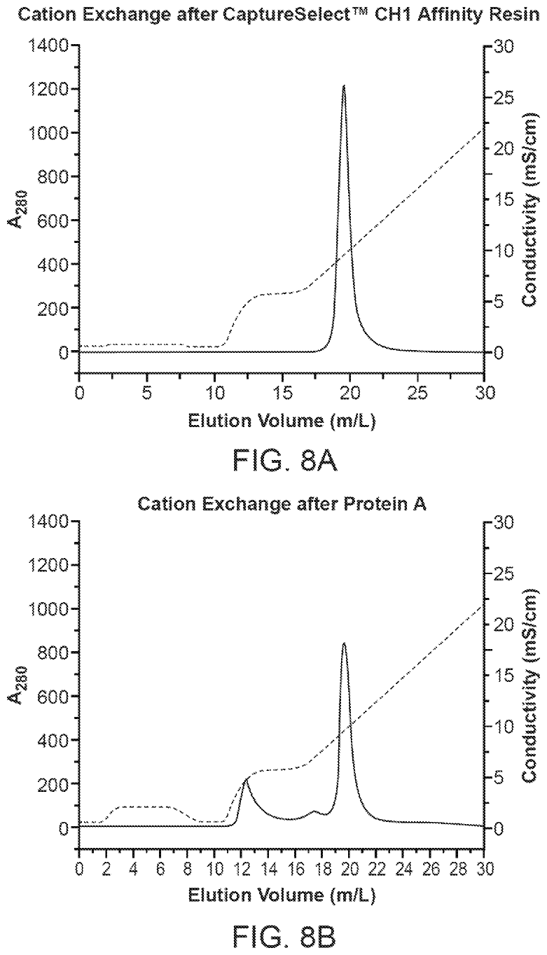

FIG. 8A is a cation exchange chromatography elution profile of “BC1” following one-step purification using the CaptureSelect™ CH1 affinity resin, showing a single tight peak. FIG. 8B is a cation exchange chromatography elution profile of “BC1” following purification using standard Protein A purification, showing additional elution peaks consistent with the co-purification of incomplete assembly products.

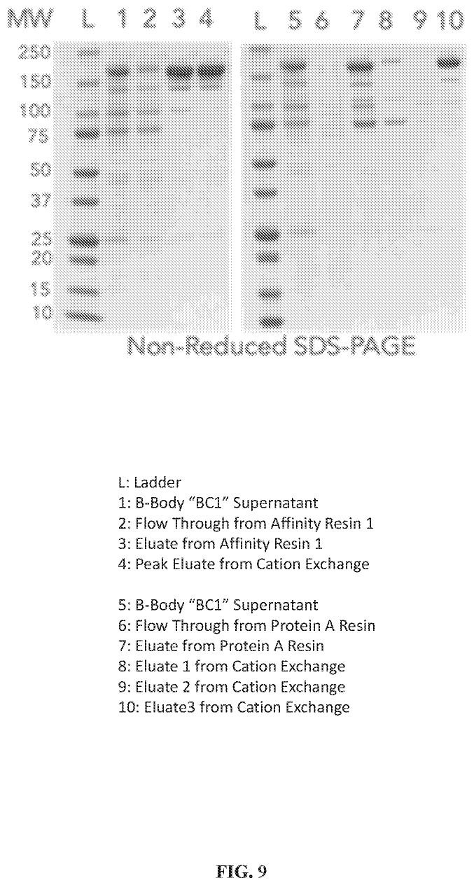

FIG. 9 shows SDS-PAGE gels under non-reducing conditions. As seen in lane 3, single-step purification of “BC1” with CH1 affinity resin provides a nearly homogeneous single band, with lane 4 showing minimal additional purification with a subsequent cation exchange polishing step. Lane 7, by comparison, shows less substantial purification using standard Protein A purification, with lanes 8-10 demonstrating further purification of the Protein A purified material using cation exchange chromatography.

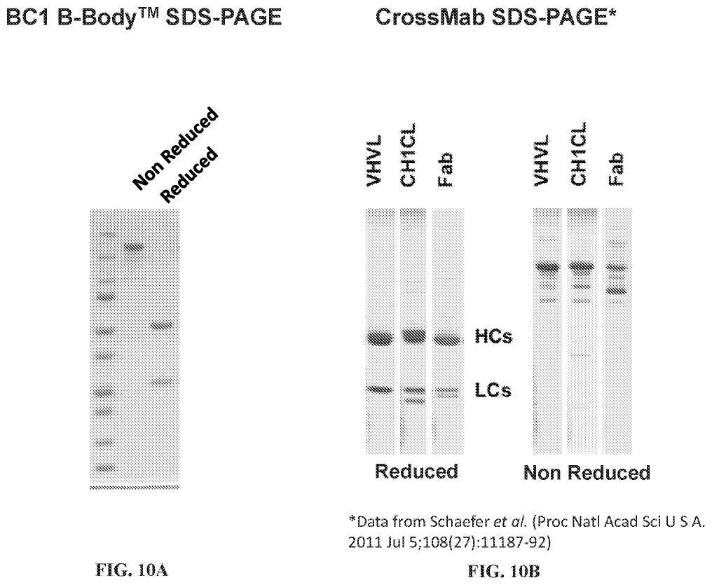

FIG. 10 compares SDS-PAGE gels of “BC1” after single-step CH1-affinity purification, under both non-reducing and reducing conditions (Panel A) with SDS-PAGE gels of a CrossMab bispecific antibody under non-reducing and reducing conditions as published in the referenced literature (Panel B).

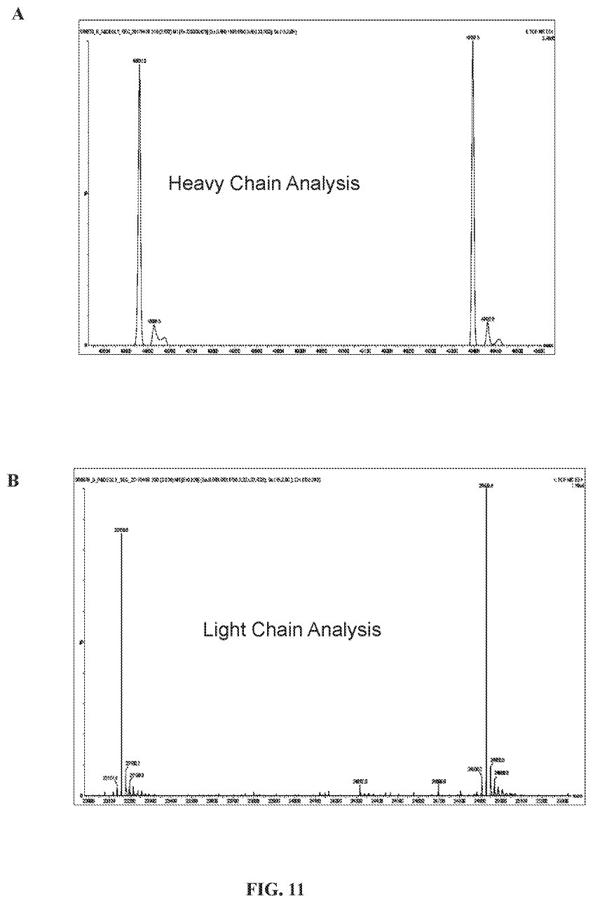

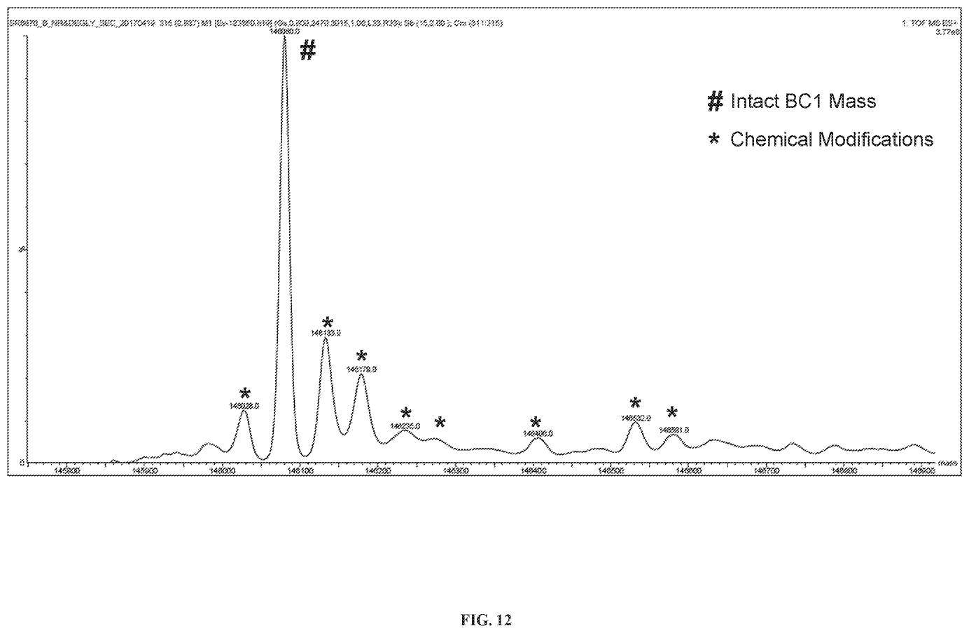

FIG. 11 shows mass spec analysis of “BC1”, demonstrating two distinct heavy chains (FIG. 11A) and two distinct light chains (FIG. 11B) under reducing conditions. The mass spectrometry data in FIG. 12 confirms the absence of incomplete pairing after purification.

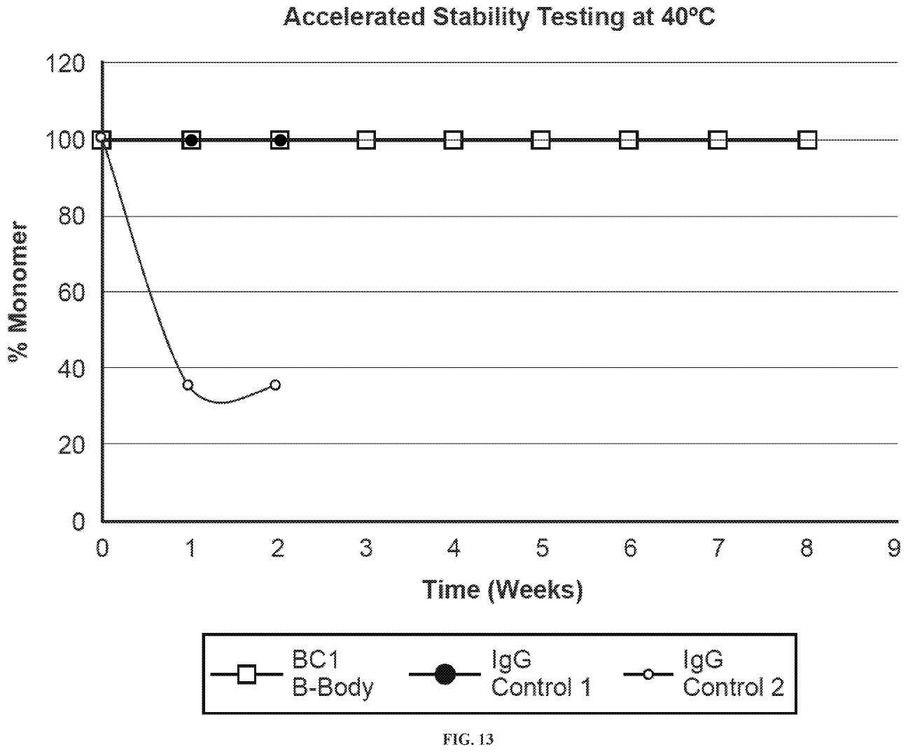

Accelerated stability testing was performed to evaluate the long-term stability of the “BC1” B-Body design. The purified B-Body was concentrated to 8.6 mg/ml in PBS buffer and incubated at 40° C. The structural integrity was measured weekly using analytical size exclusion chromatography (SEC) with a Shodex KW-803 column. The structural integrity was determined by measuring the percentage of intact monomer (% Monomer) in relation to the formation of aggregates. Data are shown in FIG. 13. The IgG Control 1 is a positive control with good stability properties. IgG Control 2 is a negative control that is known to aggregate under the incubation conditions. The “BC1” B-Body has been incubated for 8 weeks without any loss of structural integrity as determined by the analytical SEC.

We have also determined that “BC1” has high thermostability, with a TM of the bivalent construct of ˜72° C.

Table 1 compares “BC1” to CrossMab in key developability characteristics:

TABLE 1
BC1 and CrossMab Purification Analysis

| | | Roche | |
|---|---|---|---|
| Parameter | Unit | CrossMab* | “BC1” |
| Purification yield after | mg/L | 58.5 | 300 |
| protein A/SEC | | | |
| Homogeneity After purification | % SEC Area | 50-85 | 98 |
| Denaturation Temp (Tm) | degrees C. | 69.2 | 72 |

*Data from Schaefer et al. (Proc Natl Acad Sci USA. 2011 Jul. 5; 108(27): 11187-92)

### 6.13.4. Example 3: Bivalent Bispecific B-Body “BC6”

We constructed a bivalent bispecific B-Body, termed “BC6”, that is identical to “BC1” but for retaining wild type residues in Domain B at residue 366 and Domain G at residue 351. “BC6” thus lacks the charge-pair cognates T366K and L351D that had been designed to facilitate correct pairing of domain B and domain G in “BC1”. Salient features of the “BC6” architecture are illustrated in FIG. 14.

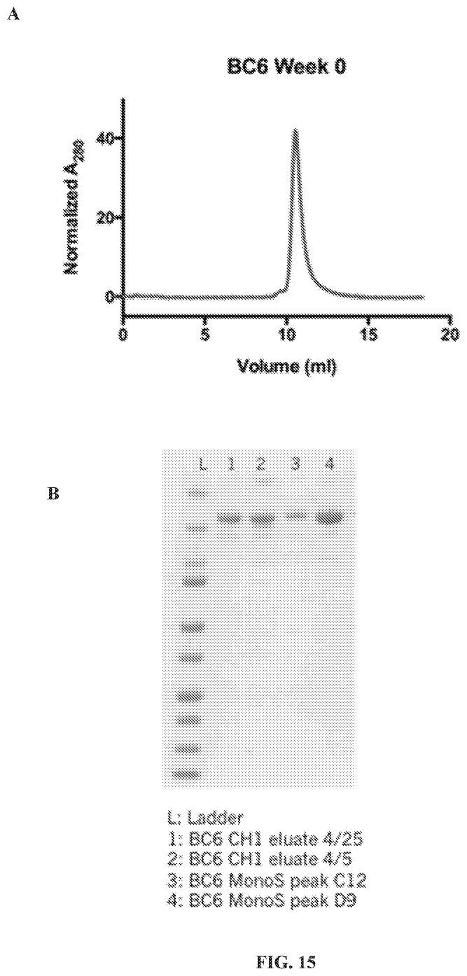

Notwithstanding the absence of the charge-pair residues present in “BC1”, we found that a single step purification of “BC6” using CH1 affinity resin resulted in a highly homogeneous sample. FIG. 15A shows SEC analysis of “BC6” following one-step purification using the CaptureSelect™ CH1 affinity resin. The data demonstrate that the single step CH1 affinity purification yields a single monodisperse peak, similar to what we observed with “BC1”, demonstrating that the disulfide bonds between polypeptide chains 1 and 2 and between polypeptide chains 3 and 4 are intact. The chromatogram also shows the absence of non-covalent aggregates.

FIG. 15B shows a SDS-PAGE gel under non-reducing conditions, with lane 1 loaded with a first lot of “BC6” after a single-step CH1 affinity purification, lane 2 loaded with a second lot of “BC6” after a single-step CH1 affinity purification. Lanes 3 and 4 demonstrate further purification can be achieved with ion exchange chromatography subsequent to CH1 affinity purification.

### 6.13.5. Example 4: Bivalent Bispecific B-Bodies “BC28”, “BC29”, “BC30”, “BC31”

We constructed bivalent 1×1 bispecific B-Bodies “BC28”, “BC29”, “BC30” and “BC31” having an engineered disulfide within the CH3 interface in Domains B and G as an alternative S-S linkage to the C-terminal disulfide present in “BC1” and “BC6”. Literature indicates that CH3 interface disulfide bonding is insufficient to enforce orthogonality in the context of Fc CH3 domains. The general architecture of these B-Body constructs is schematized in FIG. 16 with salient features of “BC28” summarized below:
Polypeptide chain 1: “BC28” chain 1 (SEQ ID NO:24)
   Domain A=VL (Antigen “A”)
   Domain B=CH3 (Y349C; 445P, 446G, 447K insertion)
   Domain D=CH2
   Domain E=CH3 (S354C, T366W)
Polypeptide chain 2: “BC28” chain 2 (SEQ ID NO:25)
   Domain F=VH (Antigen “A”)
   Domain G=CH3 (S354C; 445P, 446G, 447K insertion)
Polypeptide chain 3: “BC1” chain 3 (SEQ ID NO:10)
   Domain H=VL (“Nivo”)
   Domain I=CL (Kappa)
   Domain J=CH2
   Domain K=CH3 (Y349C, D356E, L358M, T366S, L368A, Y407V)
Polypeptide chain 4: “BC1” chain 4 (SEQ ID NO:11)
   Domain L=VH (“Nivo”)
   Domain M=CH1.

The “BC28” A:F antigen binding site is specific for “Antigen A”. The “BC28” H:L antigen binding site is specific for PD1 (nivolumab sequences). “BC28” domain B has the following changes as compared to wild type CH3: Y349C; 445P, 446G, 447K insertion. “BC28” domain E has the following changes as compared to wild type CH3: S354C, T366W. “BC28” domain G has the following changes as compared to wild type: S354C; 445P, 446G, 447K insertion.

“BC28” thus has an engineered cysteine at residue 349C of Domain B and engineered cysteine at residue 354C of domain G (“349C-354C”).

“BC29” has engineered cysteines at residue 351C of Domain B and 351C of Domain G (“351C-351C”). “BC30” has an engineered cysteine at residue 354C of Domain B and 349C of Domain G (“354C-349C”). BC31 has an engineered cysteine at residue 394C and engineered cysteine at 394C of Domain G (“394C-394C”). BC32 has engineered cysteines at residue 407C of Domain B and 407C of Domain G (“407C-407C”).

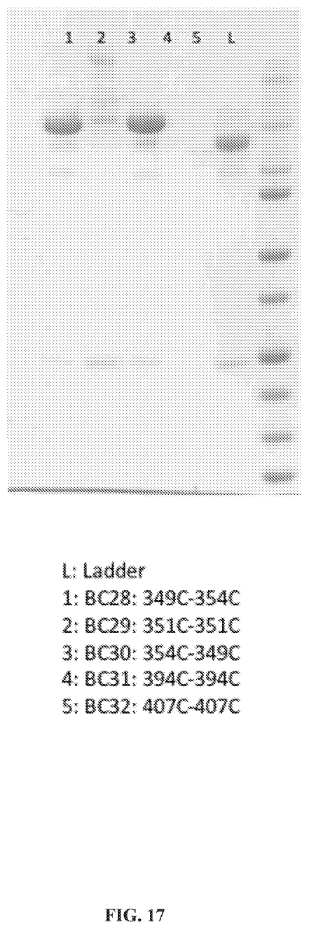

FIG. 17 shows SDS-PAGE analysis under non-reducing conditions following one-step purification using the CaptureSelect™ CH1 affinity resin. Lanes 1 and 3 show high levels of expression and substantial homogeneity of intact “BC28” (lane 1) and “BC30” (lane 3). Lane 2 shows oligomerization of BC29. Lanes 4 and 5 show poor expression of BC31 and BC32, respectively, and insufficient linkage in BC32. Another construct, BC9, which had cysteines introduced at residue 392 in domain B and 399 in Domain G (“392C-399C”), a disulfide pairing reported by Genentech, demonstrated oligomerization on SDS PAGE (data not shown).

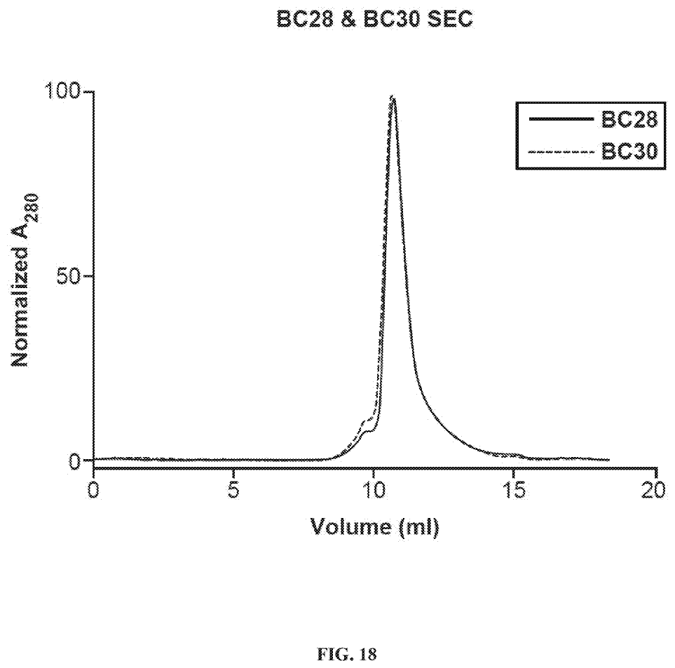

FIG. 18 shows SEC analysis of “BC28” and “BC30” following one-step purification using the CaptureSelect™ CH1 affinity resin. We have also demonstrated that “BC28” can readily be purified using a single step purification using Protein A resin (results not shown).

### 6.13.6. Example 5: Bivalent Bispecific B-Body “BC44”

FIG. 19 shows the general architecture of the bivalent bispecific 1×1 B-Body “BC44”, our currently preferred bivalent bispecific 1×1 construct.
first polypeptide chain (′BC44″ chain 1) (SEQ ID NO:32)
   Domain A=VL (Antigen “A”)
   Domain B=CH3 (P343V; Y349C; 445P, 446G, 447K insertion)
   Domain E=CH2
   Domain E=CH3 (S354C, T366W)
second polypeptide chain (=“BC28” polypeptide chain 2) (SEQ NO:25)
   Domain F=VH (Antigen “A”)
   Domain G=CH3 (S354C; 445P, 446G, 447K insertion)
third polypeptide chain (=“BC1” polypeptide chain 3) (SEQ ID NO:10)
   Domain H=VL (“Nivo”)
   Domain I=CL (Kappa)
   Domain J=CH2
   Domain K=CH3 (Y349C, D356E, L358M, T366S, L368A, Y407V)
fourth polypeptide chain (=“BC1” polypeptide chain 4) (SEQ ID NO:11)
   Domain L=VH (“Nivo”)
   Domain M=CH1.

### 6.13.7. Example 6: Variable-CH3 Junction Engineering

We produced a series of variants in which we mutated the VL-CH3 junction between Domains A and B and the VH-CH3 junction between domains F and G to assess the expression level, assembly and stability of bivalent 1×1 B-Body constructs. Although there are likely many solutions, to reduce introduction of T cell epitopes we chose to only use residues found naturally within the VL, VH and CH3 domains. Structural assessment of the domain architecture further limits desirable sequence combinations. Table 2 and Table 3 below show junctions for several junctional variants based on “BC1” and other bivalent constructs.

Variants of Variable Domain/Constant Domain Junctions for 1^{st} Polypeptide Chain

| | VL | | | | | | CH3 | | | | |
|---|---|---|---|---|---|---|---|---|---|---|---|
| Variant | 106 | 107 | 108 | 109 | 110 | 111 | 343 | 344 | 345 | 346 | Sequence |
| BC1 | I | K | R | T | | | P | R | E | P | IKRTPREP |
| | | | | | | | | | | | (SEQ ID NO: 57) |
| BC13 | I | K | R | T | | | P | R | E | P | IKRTPREP |
| | | | | | | | | | | | (SEQ ID NO: 57) |
| BC14 | I | K | R | T | | | P | R | E | P | IKRTPREP |
| | | | | | | | | | | | (SEQ ID NO: 57) |
| BC15 | I | K | R | T | V | | | R | E | P | IKRTVREP |
| | | | | | | | | | | | (SEQ ID NO: 58) |
| BC16 | I | K | R | T | | | | R | E | P | IKRTREP |
| | | | | | | | | | | | (SEQ ID NO: 59) |
| BC17 | I | K | R | T | V | | P | R | E | P | IKRTVPREP |
| | | | | | | | | | | | (SEQ ID NO: 60) |
| BC24 | I | K | R | T | | | P | R | E | P | IKRTPREP |
| | | | | | | | | | | | (SEQ ID NO: 57) |
| BC25 | I | K | R | T | | | P | R | E | P | IKRTPREP |
| | | | | | | | | | | | (SEQ ID NO: 57) |
| BC26 | I | K | R | T | V | A | | | E | P | IKRTVAEP |
| | | | | | | | | | | | (SEQ ID NO: 61) |
| BC27 | I | K | R | T | V | A | P | R | E | P | IKRTVAPREP |
| | | | | | | | | | | | (SEQ ID NO: 62) |
| BC44 | I | K | R | T | | | V | R | E | P | IKRTVREP |
| | | | | | | | | | | | (SEQ ID NO: 58) |
| BC45 | I | K | R | T | | | P | R | E | P | IKRTPREP |
| | | | | | | | | | | | (SEQ ID NO: 57) |
| BC5 | I | K | R | T | | | P | R | E | P | IKRTPREP |
| | | | | | | | | | | | (SEQ ID NO: 57) |
| BC6 | I | K | R | T | | | P | R | E | P | IKRTPREP |
| | | | | | | | | | | | (SEQ ID NO: 57) |
| BC28 | I | K | R | T | | | P | R | E | P | IKRTPREP |
| | | | | | | | | | | | (SEQ ID NO: 57) |
| BC30 | I | K | R | T | | | P | R | E | P | IKRTPREP |
| | | | | | | | | | | | (SEQ ID NO: 57) |

TABLE 3
Variants of Variable Domain/Constant Domain Junctions for 2^{nd} Polypeptide Chain

| | VH | | | | | | | CH3 | | | | |
|---|---|---|---|---|---|---|---|---|---|---|---|---|
| Variant | 112 | 113 | 114 | 115 | 116 | 117 | 118 | 343 | 344 | 345 | 346 | |
| BC1 | S | S | A | S | | | | P | R | E | P | SSASPREP |
| | | | | | | | | | | | | (SEQ ID NO: 63) |
| BC13 | S | S | A | S | T | | | R | E | P | | SSASTREP |
| | | | | | | | | | | | | (SEQ ID NO: 64) |
| BC14 | S | S | A | S | T | | | P | R | E | P | SSASTPREP |
| | | | | | | | | | | | | (SEQ ID NO: 65) |
| BC15 | S | S | A | S | | | | P | R | E | P | SSASPREP |
| | | | | | | | | | | | | (SEQ ID NO: 63) |
| BC16 | S | S | A | S | | | | P | R | E | P | SSASPREP |
| | | | | | | | | | | | | (SEQ ID NO: 63) |
| BC17 | S | S | A | S | | | | P | R | E | P | SSASPREP |
| | | | | | | | | | | | | (SEQ ID NO: 63) |
| BC24 | S | S | A | S | T | K | G | | | E | P | SSASTKGEP |
| | | | | | | | | | | | | (SEQ ID NO: 66) |
| BC25 | S | S | A | S | T | K | G | | R | E | P | SSASTKGREP |
| | | | | | | | | | | | | (SEQ ID NO: 67) |
| BC26 | S | S | A | S | | | | P | R | E | P | SSASPREP |
| | | | | | | | | | | | | (SEQ ID NO: 63) |
| BC27 | S | S | A | S | | | | P | R | E | P | SSASPREP |
| | | | | | | | | | | | | (SEQ ID NO: 63) |
| BC44 | S | S | A | S | | | | P | R | E | P | SSASPREP |
| BC45 | S | S | A | S | | | | P | R | E | P | SSASPREP |
| | | | | | | | | | | | | (SEQ ID NO: 63) |
| BC5 | S | S | A | S | | | | P | R | E | P | SSASPREP |
| | | | | | | | | | | | | (SEQ ID NO: 63) |
| BC6 | S | S | A | S | | | | P | R | E | P | SSASPREP |
| | | | | | | | | | | | | (SEQ ID NO: 63) |
| BC28 | S | S | A | S | | | | P | R | E | P | SSASPREP |
| | | | | | | | | | | | | (SEQ ID NO: 63) |
| BC30 | S | S | A | S | | | | P | R | E | P | SSASPREP |
| | | | | | | | | | | | | (SEQ ID NO: 63) |

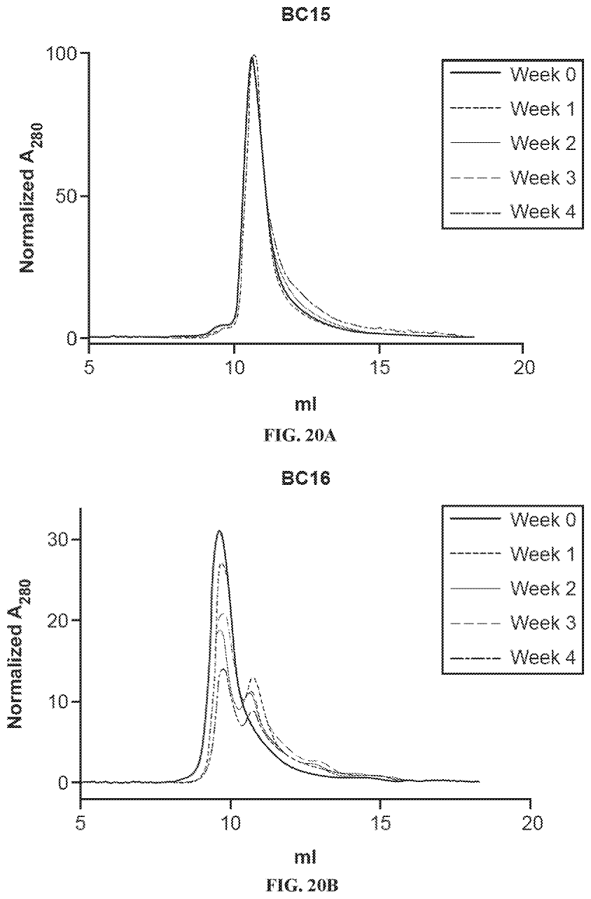

FIG. 20 shows size exclusion chromatography of “BC15” and “BC16” samples at the indicated week of an accelerated stability testing protocol at 40° C. “BC15” remained stable; “BC16” proved to be unstable over time.

### 6.13.8. Example 7: Trivalent 2×1 Bispecific B-Body Construct (“BC1-2×1”)

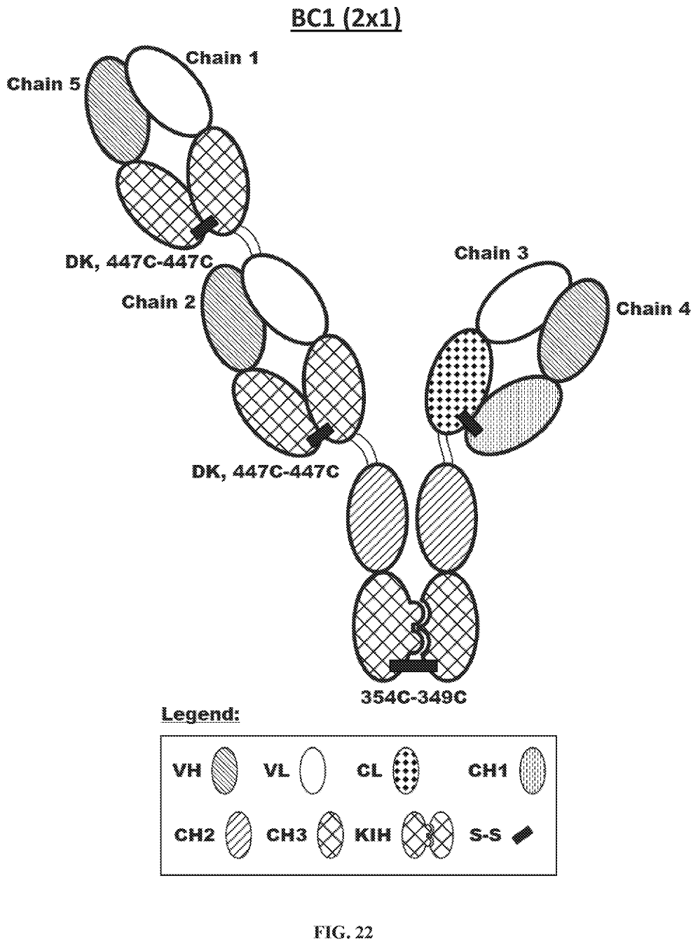

We constructed a trivalent 2×1 bispecific B-Body “BC1-2×1” based on “BC1”. Salient features of the architecture are illustrated in FIG. 22.

In greater detail, using the domain and polypeptide chain references summarized in FIG. 21,
1^{st} polypeptide chain
   Domain N=VL (“Antigen A”)
   Domain O=CH3 (T366K, 447C)
   Domain A=VL (“Antigen A”)
   Domain B=CH3 (T366K, 447C)
   Domain D=CH2
   Domain E=CH3 (Knob, 354C)
5^{th} polypeptide chain (=“BC1” chain 2)
   Domain P=VH (“Antigen A”)
   Domain Q=CH3 (L351D, 447C)
2^{nd} polypeptide chain (=“BC1” chain 2)
   Domain F=VH (“Antigen A”)
   Domain G=CH3 (L351D, 447C)
3^{rd} polypeptide chain (=“BC1” chain 3)
   Domain H=VL (“Nivo”)
   Domain I=CL (Kappa)
   Domain J=CH2
   Domain K=CH3 (Hole, 349C)
4^{th} polypeptide chain (=“BC1” chain 4)
   Domain L=VH (“Nivo”)
   Domain M=CH1.

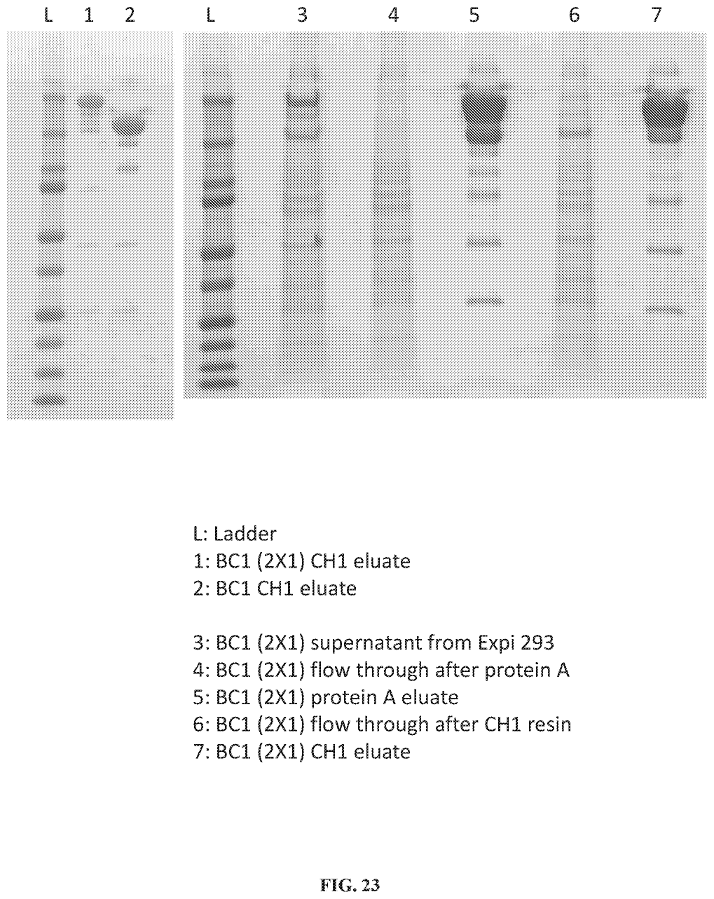

FIG. 23 shows non-reducing SDS-PAGE of protein expressed using the ThermoFisher Expi293 transient transfection system.

Lane 1 shows the eluate of the trivalent 2×1 “BC1-2×1” protein following one-step purification using the CaptureSelect™ CH1 affinity resin. Lane 2 shows the lower molecular weight, faster migrating, bivalent “BC1” protein following one-step purification using the CaptureSelect™ CH1 affinity resin. Lanes 3-5 demonstrate purification of “BC1-2×1” using protein A. Lanes 6 and 7 show purification of “BC1-2×1” using CH1 affinity resin.

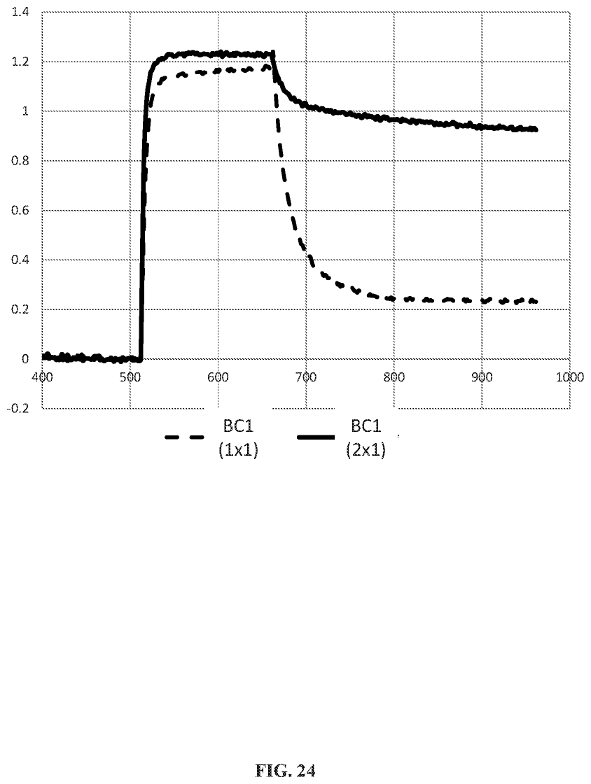

FIG. 24 compares the avidity of the bivalent “BC1” construct to the avidity of the trivalent 2×1 “BC1-2×1” construct using an Octet (Pall ForteBio) analysis. Biotinylated antigen “A” is immobilized on the surface, and the antibody constructs are passed over the surface for binding analysis.

### 6.13.9. Example 8: Trivalent 2×1 Trispecific B-Body Construct (“TB111”)

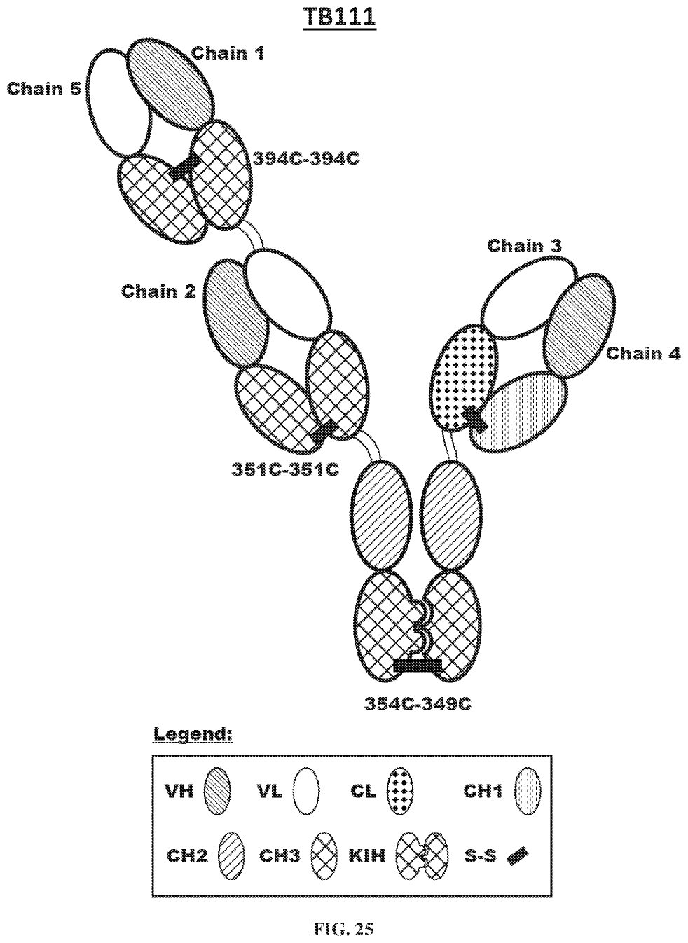

We designed a trivalent 2×1 trispecific molecule, “TB111”, having the architecture schematized in FIG. 25. With reference to the domain naming conventions set forth in FIG. 21, TB111 has the following architecture (“Ada” indicates a V region from adalimumab):
polypeptide chain 1
   Domain N: VH (“Ada”)
   Domain O: CH3 (T366K, 394C)
   Domain A: VL (“Antigen A”)
   Domain B: CH3 (T366K, 349C)
   Domain D: CH2
   Domain E: CH3 (Knob, 354C)
polypeptide chain 5
   Domain P: VL (“Ada”)
   Domain Q: CH3 (L351D, 394C)
polypeptide chain 2
   Domain F: VH (“Antigen A”)
   Domain G: CH3 (L351D, 351C)
polypeptide chain 3
   Domain H: VL (“Nivo”)
   Domain I: CL (kappa)
   Domain J: CH2
   Domain K: CH3 (Hole, 349C)
polypeptide chain 4 (=“BC1” chain 4)
   Domain L: VH (“Nivo”)
   Domain M: CH1
      This construct did not express.

### 6.13.10. Example 9: Trivalent 1×2 Bispecific Construct (“BC28-1×2”)

We constructed a trivalent 1×2 bispecific B-Body having the following domain structure:
1^{st} polypeptide chain (=“BC28” chain 1) (SEQ ID NO:24)
   Domain A=VL (Antigen “A”)
   Domain B=CH3 (Y349C; 445P, 446G, 447K insertion)
   Domain D=CH2
   Domain E=CH3 (S354C, T366W)
2^{nd} polypeptide chain (=“BC28” chain 2) (SEQ ID NO:25)
   Domain F=VH (Antigen “A”)
   Domain G=CH3 (S354C; 445P, 446G, 447K insertion)
3^{rd} polypeptide chain (SEQ ID NO:37)
   Domain R=VL (Antigen “A”)
   Domain S=CH3 (Y349C; 445P, 446G, 447K insertion)
   Linker=GSGSGS
   Domain H=VL (“Nivo”)
   Domain I=CL
   Domain J=CH2
   Domain K=CH3 (Y349C, D356E, L358M, T366S, L368A, Y407V)
4^{th} polypeptide chain (=“BC1” chain 4) (SEQ ID NO:11):
   Domain L=VH (“Nivo”)
   Domain M=CH1.
6^{th} polypeptide chain (=“BC28” chain 2) (SEQ ID NO:25)
   Domain T=VH (Antigen “A”)
   Domain U=CH3 (S354C; 445P, 446G, 447K insertion)

The A:F antigen binding site is specific for “Antigen A”, as is the H:L binding antigen binding site. The R:T antigen binding site is specific for PD. The specificity of this construct is thus Antigen “A”×(PD1-Antigen “A”).

### 6.13.11. Example 10: Trivalent 1×2 Bispecific Construct (“CTLA4-4×Nivo×CTLA4-4”)

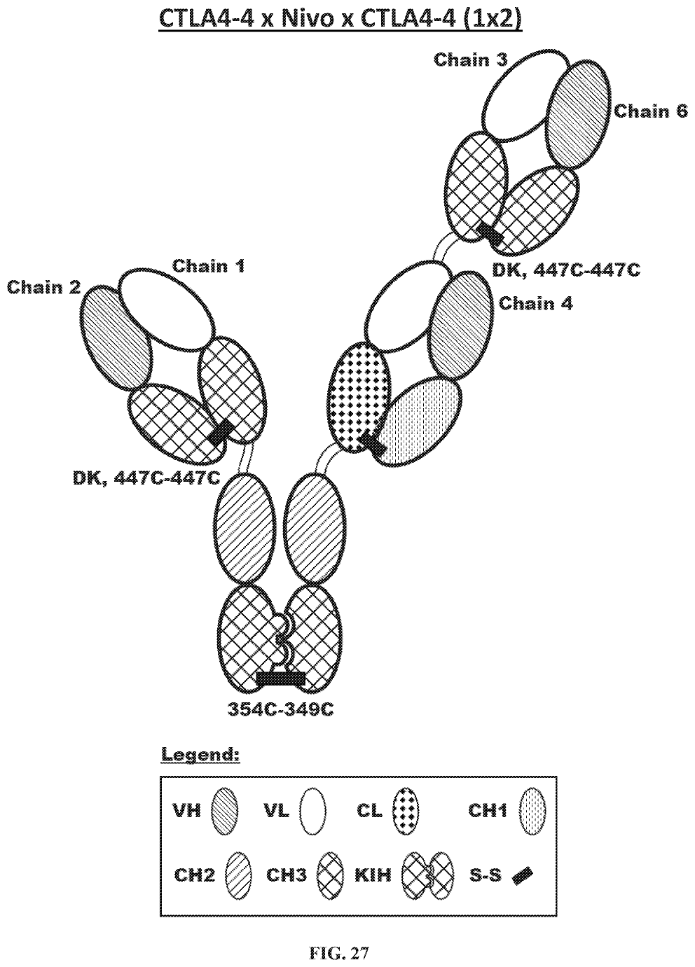

We constructed a trivalent 1×2 bispecific molecule having the general structure schematized in FIG. 27 (“CTLA4-4×Nivo×CTLA4-4”). Domain nomenclature is set forth in FIG. 26.

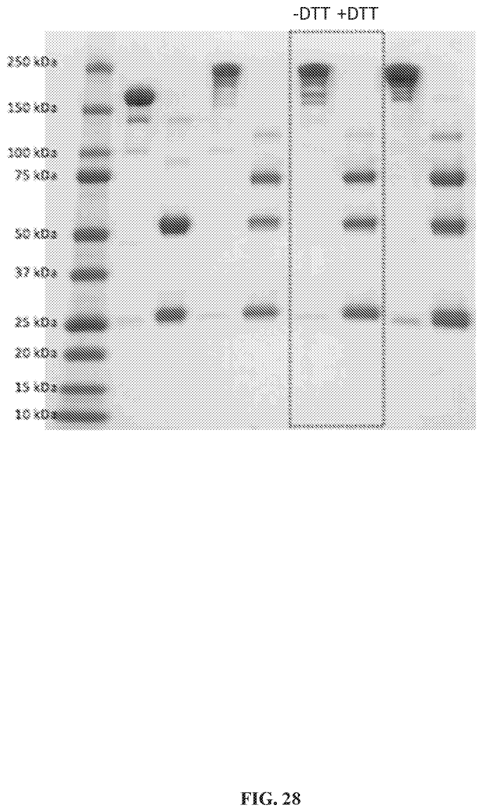

FIG. 28 is a SDS-PAGE gel in which the lanes showing the “CTLA4-4×Nivo×CTLA4-4” construct under non-reducing and reducing conditions have been boxed.

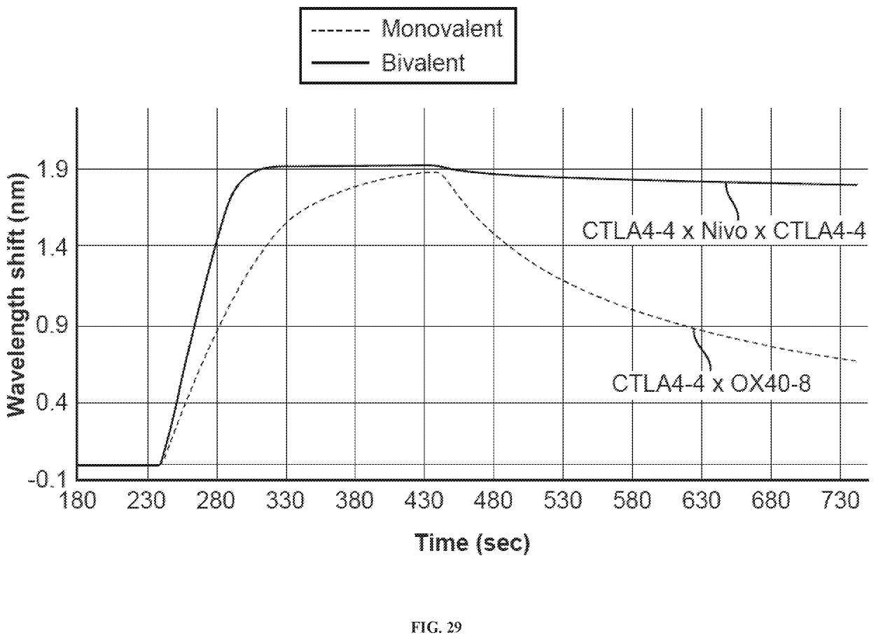

FIG. 29 compares antigen binding of two antibodies: “CTLA4-4×OX40-8” and “CTLA4-4×Nivo×CTLA4-4”. “CTLA4-4×OX40-8” binds to CTLA4 monovalently; while “CTLA4-4×Nivo×CTLA4-4” bind to CTLA4 bivalently.

### 6.13.12. Example 11: Trivalent 1×2 Trispecific Construct “BC28-1×1×1a”

We constructed a trivalent 1×2 trispecific molecule having the general structure schematized in FIG. 30. With reference to the domain nomenclature set forth in FIG. 26,
1^{st} polypeptide chain (=“BC28” chain1) [SEQ ID NO:24]
   Domain A=VL (Antigen “A”)
   Domain B=CH3 (Y349C; 445P, 446G, 447K insertion)
   Domain D=CH2
   Domain E=CH3 (S354C, T366W)
2^{nd} polypeptide chain (=“BC28” chain 2) (SEQ ID NO:25)
   Domain F=VH (Antigen “A”)
   Domain G=CH3 (S354C; 445P, 446G, 447K insertion)
3^{rd} polypeptide chain (SEQ ID NO:45)
   Domain R=VL (CTLA4-4)
   Domain S=CH3 (T366K; 445K, 446S, 447C insertion)
   Linker=GSGSGS
   Domain H=VL (“Nivo”)
   Domain I=CL
   Domain J=CH2
   Domain K=CH3 (Y349C, D356E, L358M, T366S, L368A, Y407V)
4^{th} polypeptide chain (=“BC1” chain 4) (SEQ ID NO:11)
   Domain L=VH (“Nivo”)
   Domain M=CH1.
6^{th} polypeptide chain (=hCTLA4-4 chain2) (SEQ ID NO:53)
   Domain T=VH (CTLA4)
   Domain U=CH3 (L351D, 445G, 446E, 447C insertion)

The antigen binding sites of this trispecific construct were:
Antigen binding site A:F was specific for “Antigen A”
Antigen binding site H:L was specific for PD1 (nivolumab sequence)
Antigen binding site R:T was specific for CTLA4.

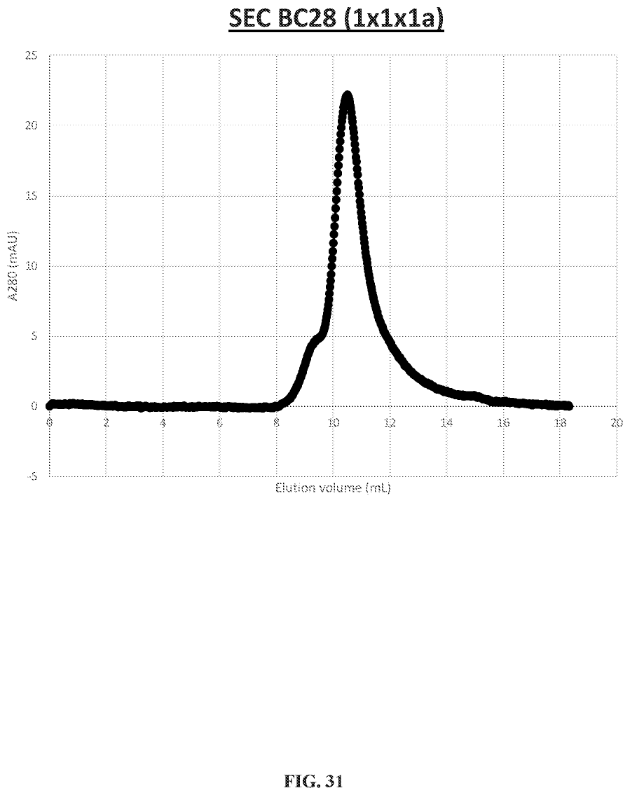

FIG. 31 shows size exclusion chromatography with “BC28-1×1×1a” following transient expression and one-step purification using the CaptureSelect™ CH1 affinity resin, demonstrating a single well-defined peak.

### 6.13.13. Example 12: SDS-PAGE Analysis of Bivalent and Trivalent Constructs

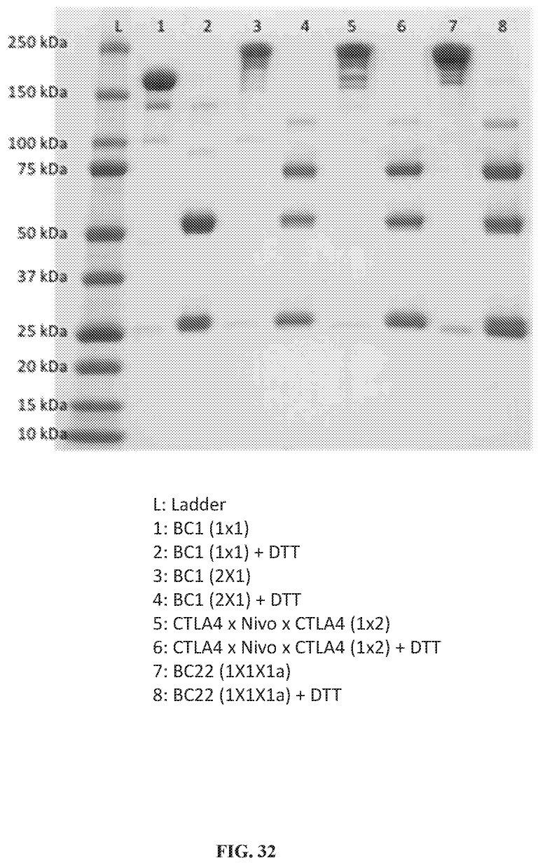

FIG. 32 shows a SDS-PAGE gel with various constructs, each after transient expression and one-step purification using the CaptureSelect™ CH1 affinity resin, under non-reducing and reducing conditions.

Lanes 1 (nonreducing conditions) and 2 (reducing conditions, +DTT) are the bivalent 1×1 bispecific construct “BC1”. Lanes 3 (nonreducing) and 4 (reducing) are the trivalent bispecific 2×1 construct “BC1-2×1” (see Example 7). Lanes 5 (nonreducing) and 6 (reducing) are the trivalent 1×2 bispecific construct “CTLA4-4×Nivo×CTLA4-4” (see Example 10). Lanes 7 (nonreducing) and 8 (reducing) are the trivalent 1×2 trispecific “BC28-1×1×1a” construct described in Example 11.

The SDS-PAGE gel demonstrates the complete assembly of each construct, with the predominant band in the non-reducing gel appearing at the expected molecular weight for each construct.

### 6.13.14. Example 13: Binding Analysis

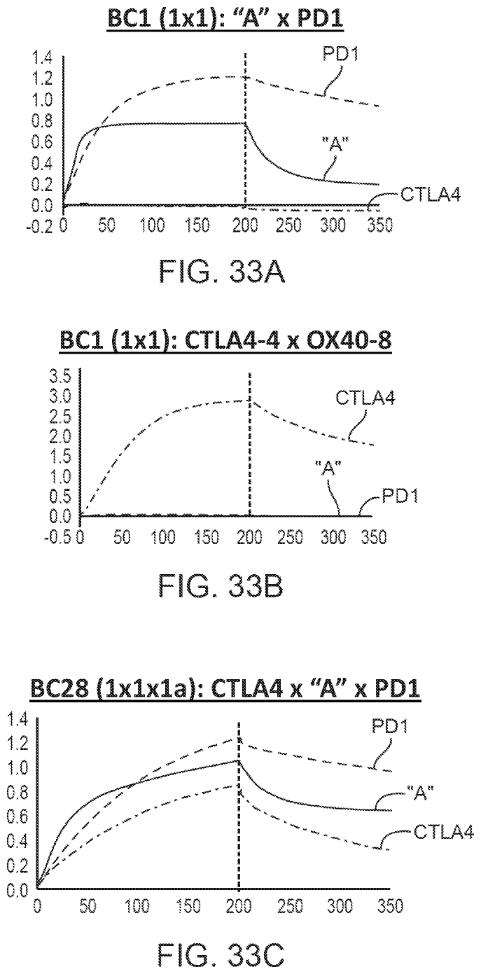

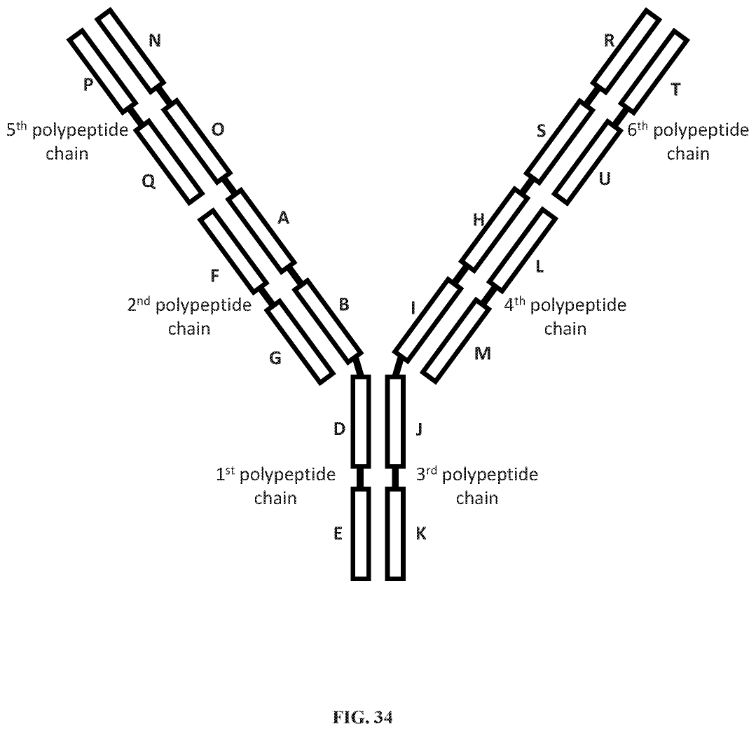

FIG. 33 shows Octet binding analyses to 3 antigens: PD1, Antigen “A”, and CTLA-4. In each instance, the antigen is immobilized and the B-Body is the analyte. For reference, 1×1 bispecifics “BC1” and “CTLA4-4×OX40-8” were also compared to demonstrate 1×1 B-Bodies bind specifically only to antigens for which the antigen binding sites were selected.

FIG. 33A shows binding of “BC1” to PD1 and to Antigen “A”, but not CTLA4. FIG. 33B shows binding of a bivalent bispecific 1×1 construct “CTLA4-4×OX40-8” to CTLA4, but not to Antigen “A” or PD1. FIG. 33C shows the binding of the trivalent trispecific 1×2 construct, “BC28-1×1×1a” to PD1, Antigen “A”, and CTLA4.

### 6.13.15. Example 14: Tetravalent Constructs

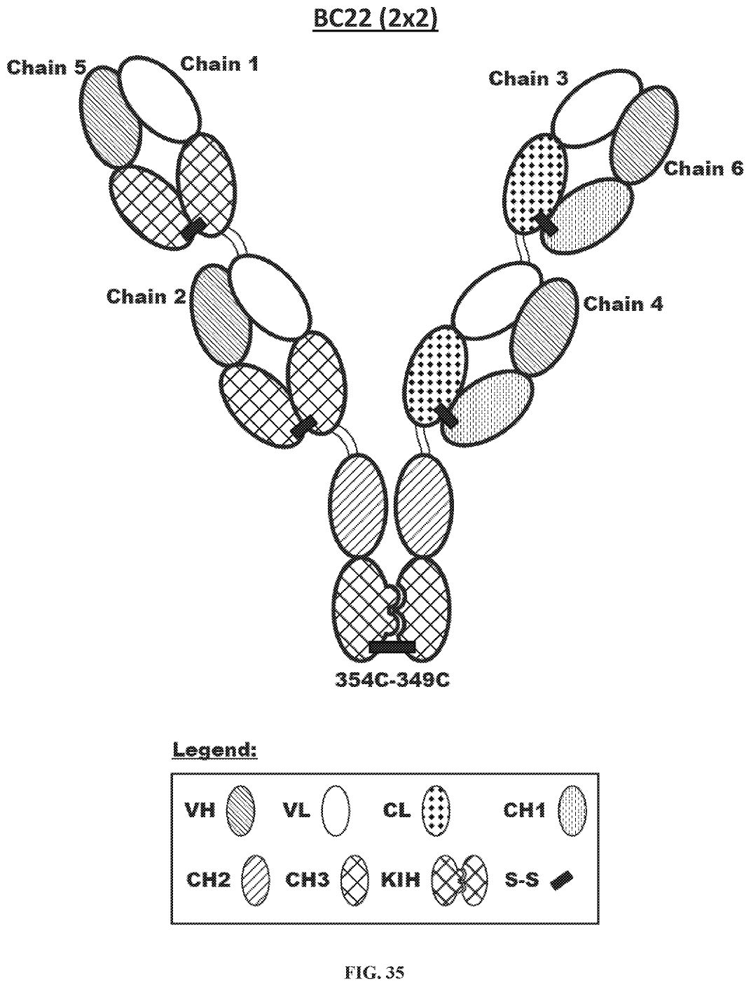

FIG. 35 shows the overall architecture of a 2×2 tetravalent bispecific construct “BC22-2×2”. The 2×2 tetravalent bispecific was constructed with “BC1” scaffold by duplicating each variable domain-constant domain segment. Domain nomenclature is schematized in FIG. 34.

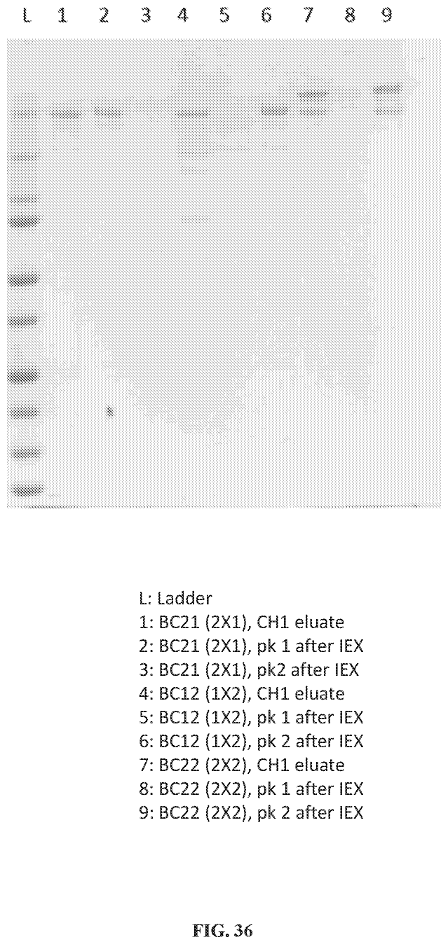

FIG. 36 is a SDS-PAGE gel. Lanes 7-9 show the “BC22-2×2” tetravalent construct respectively following one-step purification using the CaptureSelect™ CH1 affinity resin (“CH1 eluate”), and after an additional ion exchange chromatography purification (lane 8, “pk 1 after IEX”; lane 9, “pk 2 after IEX”). Lanes 1-3 are the trivalent 2×1 construct “BC21-2×1” after CH1 affinity purification (lane 1) and, in lanes 2 and 3, subsequent ion exchange chromatography. Lanes 4-6 are the 1×2 trivalent construct “BC12-1×2”.

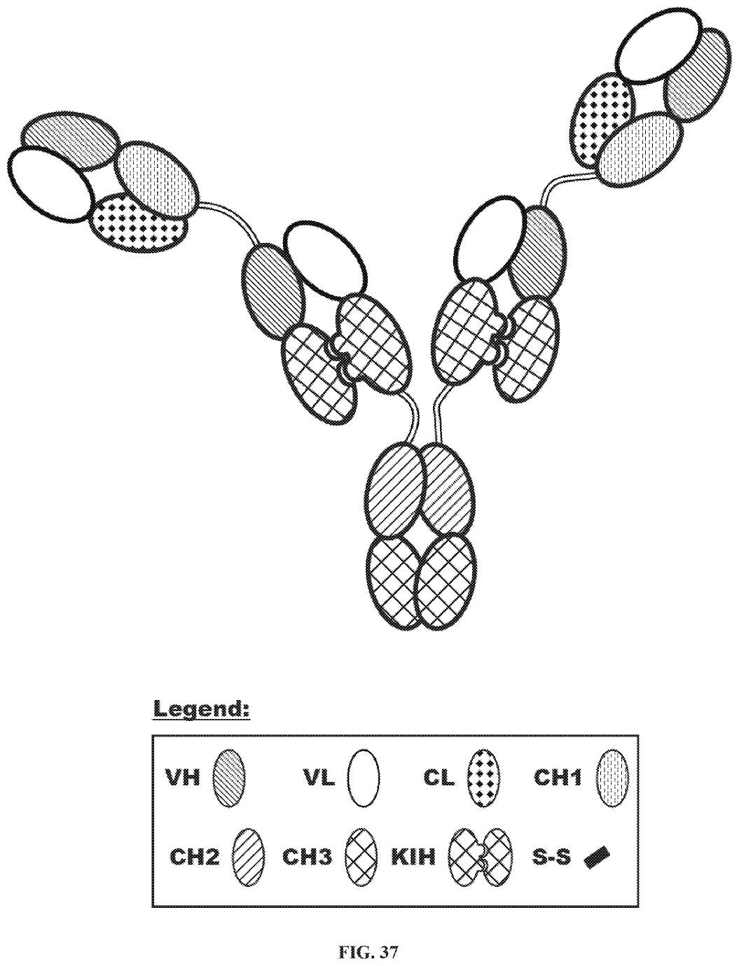

FIG. 37 shows the overall architecture of a 2×2 tetravalent construct.

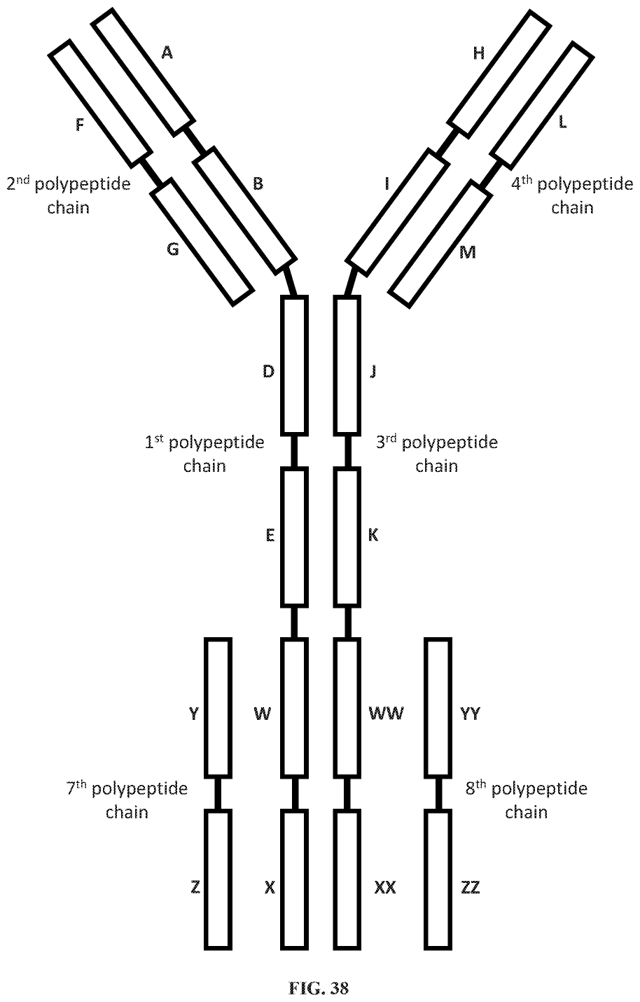

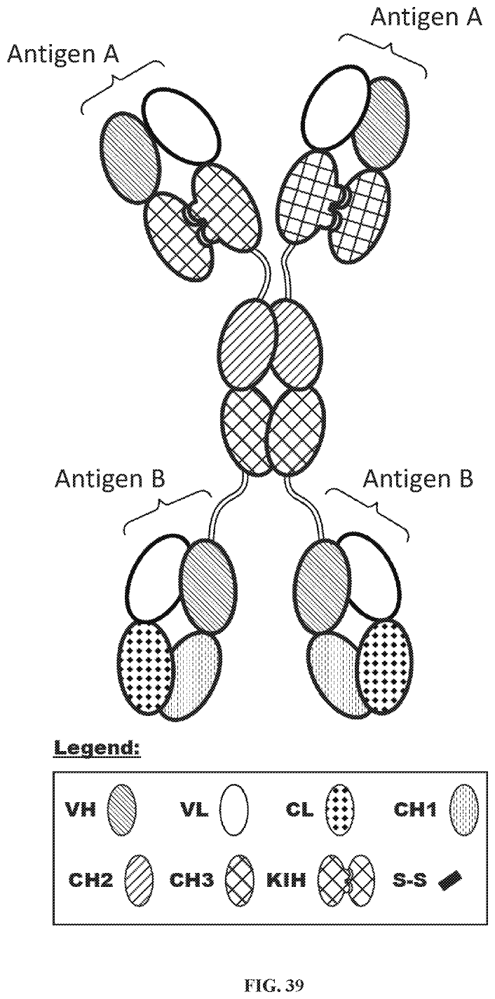

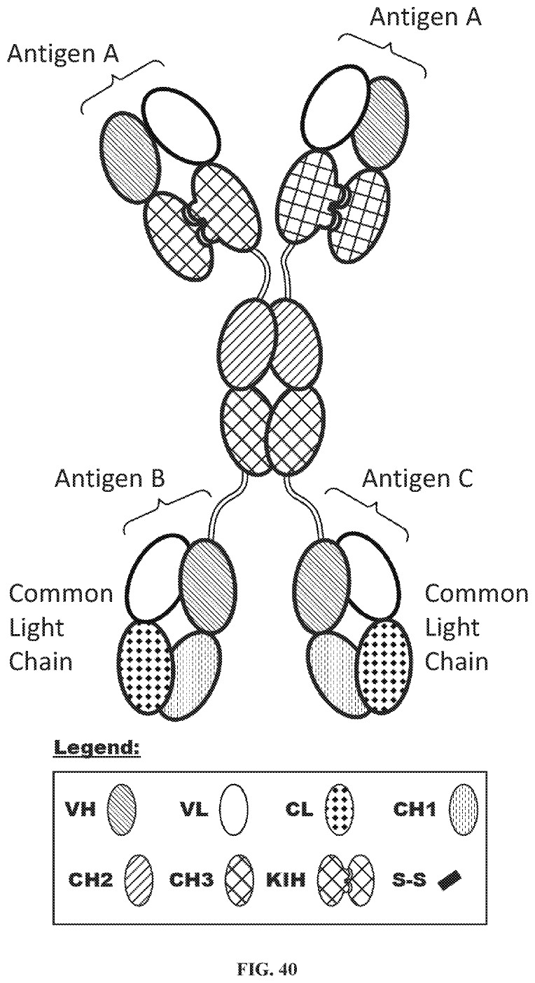

FIGS. 39 and 40 schematize tetravalent constructs having alternative architectures. Domain nomenclature is presented in FIG. 38.

### 6.13.16. Example 15: Bispecific Antigen Engagement by B-Body

A tetravalent bispecific 2×2 B-Body “B-Body-IgG 2×2” was constructed. In greater detail, using the domain and polypeptide chain references summarized in FIG. 38,
1^{st} polypeptide chain
   Domain A=VL (Certolizumab)
   Domain B=CH3 (IgG1, knob)
   Domain D=CH2 (IgG1)
   Domain E=CH3 (IgG1)
   Domain W=VH (Antigen “A”)
   Domain X=CH1 (IgG1)
3^{rd} polypeptide chain (identical to first polypeptide chain)
   Domain H=VL (Certolizumab)
   Domain I=CH3 (IgG1, knob)
   Domain J=CH2 (IgG1)
   Domain K=CH3 (IgG1)
   Domain WW=VH (Antigen “A”)
   Domain XX=CH1 (IgG1)
2^{nd} polypeptide chain
   Domain F=VH (Certolizumab)
   Domain G=CH3 (IgG1, hole)
4^{th} polypeptide chain (identical to third polypeptide chain)
   Domain F=VH (Certolizumab)
   Domain G=CH3 (IgG1, hole)
7^{th} polypeptide chain
   Domain Y=VH (“Antigen A”)
   Domain Z=CL Kappa
8^{th} polypeptide chain (identical to seventh polypeptide chain)
   Domain YY=VH (“Antigen A”)
   Domain ZZ=CL Kappa.

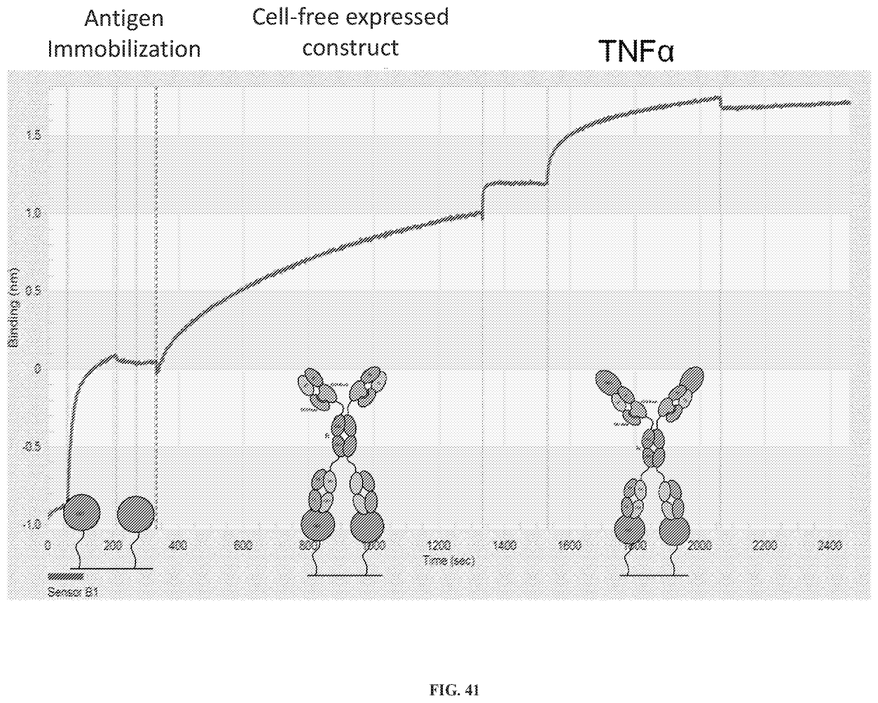

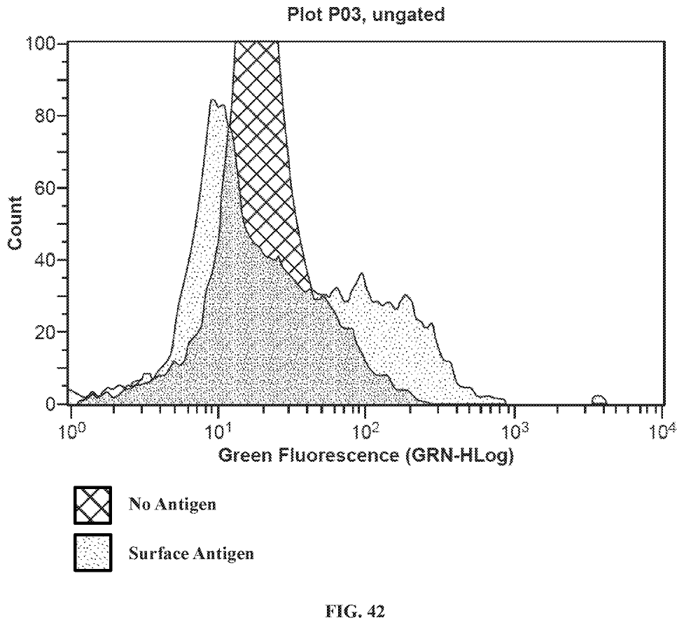

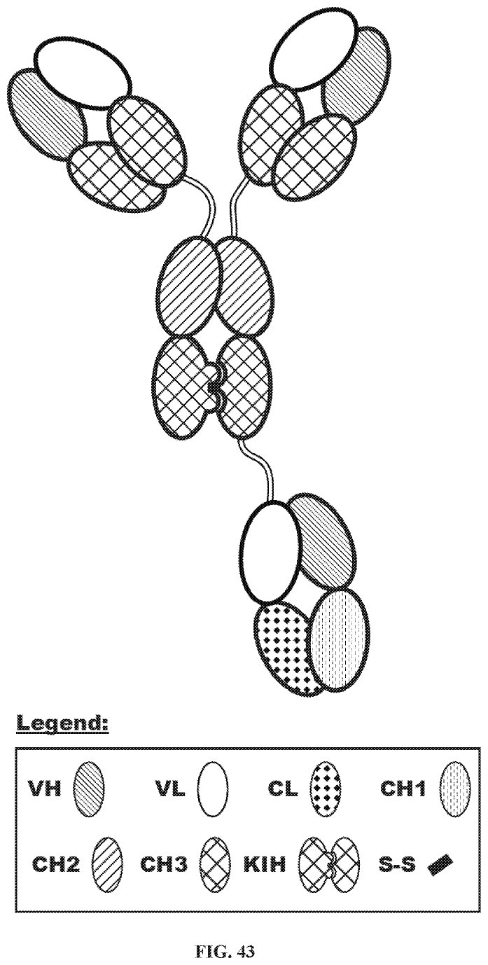

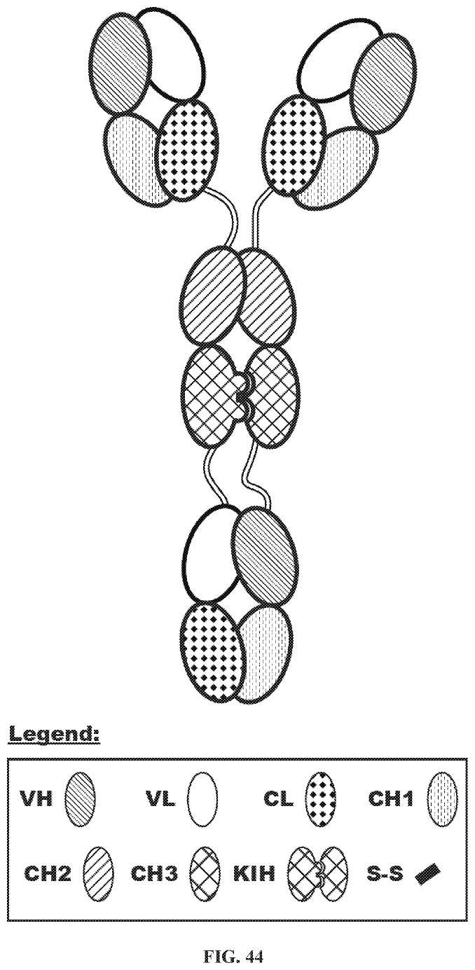

This was cloned and expressed as described in Example 1. Here, the BLI experiment consisted of immobilization of biotinylated antigen “A” on a streptavidin sensor, followed by establishing baseline with 10× kinetic buffer. The sensor was then dipped in cell-free expressed “B-Body-IgG 2×2” followed by establishment of a new baseline. Finally, the sensor was dipped in 100 nM TNFα where a second binding event was observed, confirming the bispecific binding of both antigens by a single “B-Body-IgG 2×2” construct. Results are shown in FIG. 41.

### 6.13.17. Example 16: Antigen-Specific Cell Binding of “BB-IgG 2×2”

Expi-293 cells were either mock transfected or transiently transfected with Antigen “B” using the Expi-293 Transfection Kit (Life Technologies). Forty-eight hours after transfection, the Expi-293 cells were harvested and fixed in 4% paraformaldehyde for 15 minutes at room temperature. The cells were washed twice in PBS. 200,000 Antigen B or Mock transfected Expi-293 cells were placed in a V-bottom 96 well plate in 100 μL of PBS. The cells were incubated with the “B-Body-IgG 2×2” at a concentration of 3 ug/mL for 1.5 hours at room temperature. The cells were centrifuged at 300×G for 7 minutes, washed in PBS, and incubated with 100 μL of FITC labeled goat-anti human secondary antibody at a concentration of 8 μg/mL for 1 hour at room temperature. The cells were centrifuged at 300×G for 7 minutes, washed in PBS, and cell binding was confirmed by flow cytometry using a Guava easyCyte. Results are shown in FIG. 42.

### 6.13.18. Example 17: SDS-PAGE Analysis of Bivalent and Trivalent Constructs

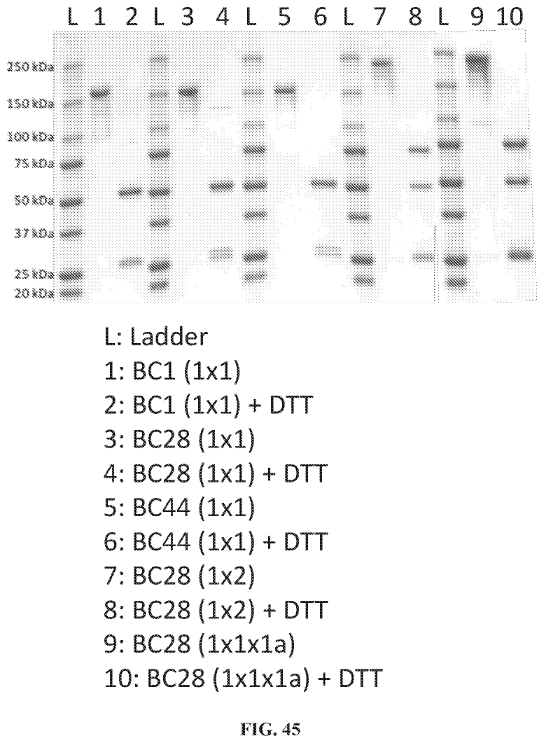

FIG. 45 shows a SDS-PAGE gel with various constructs, each after transient expression and one-step purification using the CaptureSelect™ CH1 affinity resin, under non-reducing and reducing conditions.

Lanes 1 (nonreducing conditions) and 2 (reducing conditions, +DTT) are the bivalent 1×1 bispecific construct “BC1”. Lanes 3 (nonreducing) and 4 (reducing) are the bivalent 1×1 bispecific construct “BC28” (see Example 4). Lanes 5 (nonreducing) and 6 (reducing) are the bivalent 1×1 bispecific construct “BC44” (see Example 5). Lanes 7 (nonreducing) and 8 (reducing) are the trivalent 1×2 bispecific “BC28-1×2” construct (see Example 9). Lanes 9 (nonreducing) and 10 (reducing) are the trivalent 1×2 trispecific “BC28-1×1×1a” construct described in Example 11.

The SDS-PAGE gel demonstrates the complete assembly of each construct, with the predominant band in the non-reducing gel appearing at the expected molecular weight for each construct.

### 6.13.19. Example 18: Stability Analysis of Variable-CH3 Junction Engineering

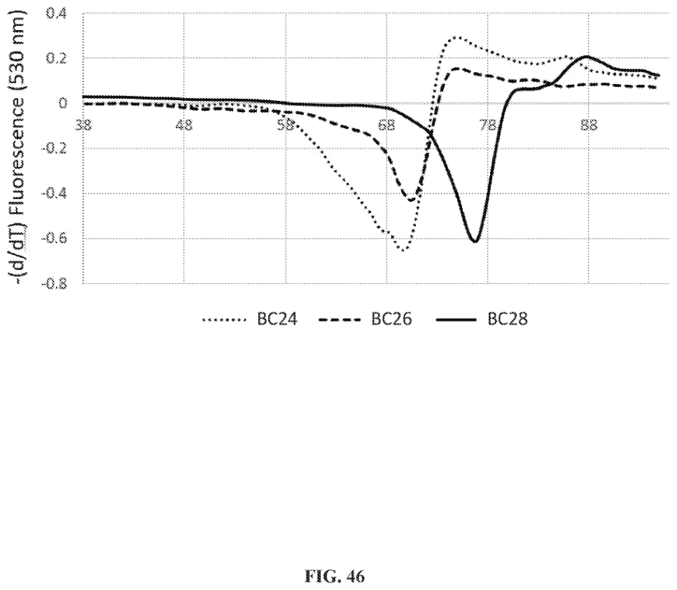

Pairing stability between various junctional variant combinations was assessed. Differential scanning fluorimetry was performed to determine the melting temperature of various junctional variant pairings between VL-CH3 polypeptides from Chain 1 (domains A and B) and VH-CH3 polypeptides from 2 (domains F and G). Junctional variants “BC6jv”, “BC28jv”, “BC30jv”, “BC44jv”, and “BC45jv”, each having the corresponding junctional sequences of “BC6”, “BC28”, “BC30”, “BC44”, and “BC45” found in Table 2 and Table 3 above, demonstrate increased pairing stability with Tm's in the 76-77 degree range (see Table 4). FIG. 46 shows differences in the thermal transitions for “BC24jv”, “BC26jv”, and “BC28jv”, with “BC28jv” demonstrating the greatest stability of the three. The x-axis of the figure is temperature and the y-axis is the change in fluorescence divided by the change in temperature (−dFluor/dTemp). Experiments were performed as described in Niesen et al. (Nature Protocols, (2007) 2, 2212-2221), which is hereby incorporated by reference for all it teaches.

TABLE 4
Melting Temperatures of Junctional Variant Pairs

| JUNCTIONAL | MELTING TEMP | MELTING TEMP |
|---|---|---|
| VARIANT PAIR | #1 (° C.) | #2 (° C.) |
| BC1jv | 69.7 | 55.6 |
| BC5jv | 71.6 | |
| BC6jv | 77 | |
| BC15jv | 68.2 | 54 |
| BC16jv | 65.9 | |
| BC17jv | 68 | |
| BC24jv | 69.7 | |
| BC26jv | 70.3 | |
| BC28jv | 76.7 | |
| BC30jv | 76.8 | |
| BC44jv | 76.2 | |
| BC45jv | 76 | |

### 6.13.20. Example 19: CD3 Candidate Binding Molecules

Various CD3 antigen binding sites were constructed and tested as described below.

### 6.13.20.1. CD3 Binding Arm

A series of CD3 binding arm variants based on a humanized version of the SP34 anti-CD3 antibody (SP34-89, SEQ ID NOs:68 and 69) were engineered with point mutations in either the VH or VL amino acid sequences (SEQ ID Nos:70-73). The various VH and VL sequences were paired together as described in Table 5.

TABLE 5
Anti-CD3 SP34 Binding Arm Variants

| | SP34-89 VL-WT | SP34-89 VL-W57G |
|---|---|---|
| VL/VH Variants | (SEQ ID NO: 69) | (SEQ ID NO: 73) |
| SP34-89 VH-WT | SP34-89 VL-WT/ | SP34-89 VL-W57G/ |
| SEQ ID NO: 68 | SP34-89 VH-WT | SP34-89 VH-WT |
| SP34-89 VH-N30S | SP34-89 VL-WT/ | SP34-89 VL-W57G/ |
| SEQ ID NO: 70 | SP34-89 VH-N30S | SP34-89 VH-N30S |
| SP34-89 VH-G65D | SP34-89 VL-WT/ | SP34-89 VL-W57G/ |
| SEQ ID NO: 71 | SP34-89 VH-G65D | SP34-89 VH-G65D |
| SP34-89 VH-S68T | SP34-89 VL-WT/ | SP34-89 VL-W57G/ |
| SEQ ID NO: 72 | SP34-89 VH-S68T | SP34-89 VH-S68T |

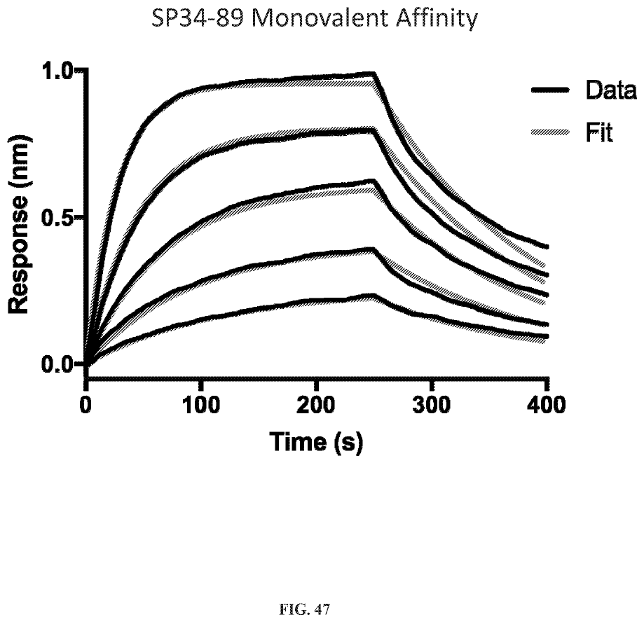

The VL and VH variants were cloned into one arm of a 1×1 BC1 B-Body, while the other arm contained an irrelevant antigen binding site. FIG. 47 demonstrates binding affinity of the non-mutagenized SP34-89 monovalent B-Body as determined by Octet (Pall ForteBio) biolayer interferometry analysis. A two-fold serial dilution (200-12.5 nM) of the construct was used to determine a binding affinity of 23 nM for SP34-89 (kₒₙ=3×10⁵M⁻¹ s⁻¹, k_{off}=7.1×10⁻³ s⁻¹), matching the affinity for other SP34 variants in the literature. The kinetic affinity also matched the equilibrium binding affinity.

### 6.13.20.2. CD3 Binding Arm Discovery

A chemically synthetic Fab phage library with diversity introduced into the Fab CDRs was screened against CD3 antigens using a monoclonal phage ELISA format where plate-immobilized CD3 variants were assessed for binding to phage, as described above. Phage clones expressing Fabs that recognized CD3 antigens were sequenced. Table A lists CD3 antigen binding site candidates. CD3-8 interestingly was cross-reactive with human and cyno CD3 antigen.

TABLE A
CD3 Antigen Binding Site Candidates

| ABS | CDRH1 | CDR H2 | CDR H3 | CDR L1 | CDR L2 | CDR L3 |
|---|---|---|---|---|---|---|
| 12B-4 | FYTYSI | YISSYSSYTY | IRLDVL | RASQSVSSAVA | SASSLYS | STSTPY |
| 12B-5 | FSSYYI | SIASLSGQTS | GAEGGM | RASQSVSSAVA | SASSLYS | WGSSLA |
| 12B-6 | FSYYFI | SIDPDFGSTY | AFFTVM | RASQSVSSAVA | SASSLYS | SSSTPR |
| 12B-8 | FKSYYI | GITSYDGYTS | GYYGGM | RASQSVSSAVA | SASSLYS | WGRLLW |
| 12B-9 | FSLYYI | GIWPHAGYTY | SYSISVL | RASQSVSSAVA | SASSLYS | AYTYPY |
| 12B-11 | FSHYSI | YIYPQDDYTE | SIGYGAM | RASQSVSSAVA | SASSLYS | FRSYLR |
| 12B-12 | FYSYYI | SIDPYFGDTT | AHFLAYGL | RASQSVSSAVA | SASSLYS | RSSDLY |
| 12B-13 | FSSYLI | WIYPYDDYTY | DFGFMHGF | RASQSVSSAVA | SASSLYS | WYSSLS |
| 12B-14 | FSSYYI | YIYPYDGYTK | YSYGSFGL | RASQSVSSAVA | SASSLYS | WYSSPV |
| 12B-16 | FLFYRI | QIYPQSGYTS | ADSYRSAF | RASQSVSSAVA | SASSLYS | SWDDLR |
| 12B-17 | FSSYYI | WIYSSGSYTS | GIFSGYGL | RASQSVSSAVA | SASSLYS | RYTSLV |
| 12B-18 | FSYYYI | YISSYRGSTA | SSSYLRIRY | RASQSVSSAVA | SASSLYS | AITSLL |
| 12B-19 | FSSYRI | YIAPYAGYTY | GYYLGQGAF | RASQSVSSAVA | SASSLYS | AISTLW |
| 12B-20 | FVYYYI | WIYSTGGGTS | GYYLTYTGL | RASQSVSSAVA | SASSLYS | LDDSLF |
| 12B-21 | FSYYSI | YISPYKGYTY | YTSYSREAM | RASQSVSSAVA | SASSLYS | SKRVPL |
| 12B-22 | FGYYDI | YISPGGGSTG | LGSLRYYVF | RASQSVSSAVA | SASSLYS | WYSSLL |
| 12B-24 | FSSYGI | GIDPYGTYTS | HFGTGRYGGL | RASQSVSSAVA | SASSLYS | SDSVPL |
| 12B-25 | FSSYGI | YIYPSWGYTV | YRPGVYMYGL | RASQSVSSAVA | SASSLYS | AHSSLP |
| 12B-26 | FTGYYI | SIYPDGGSTI | GGAGSRLVV | RASQSVSSAVA | SASSLYS | LYSSLW |
| 12B-27 | FSSYYI | WISSSGSHTS | GSSHTFFDAL | RASQSVSSAVA | SASSLYS | RGSSLL |
| 12B-29 | FRFYDI | AIYPTRSYTW | GAPFSGYSGM | RASQSVSSAVA | SASSLYS | ASRIPL |
| 12B-30 | FKSYYI | LIDPYSGITT | PSGASALQAM | RASQSVSSAVA | SASSLYS | APSWLA |
| 12B-31 | FPYYLI | VIQPYSSYTA | ESAGYFYGGL | RASQSVSSAVA | SASSLYS | FGSTLY |
| 12B-33 | FPGYSI | YIYPYGGYTY | FSRSRYGVGM | RASQSVSSAVA | SASSLYS | TDSLPL |
| 12B-34 | FDSYII | YITPDIDITY | ASSWTFFEAF | RASQSVSSAVA | SASSLYS | SITSLS |
| 12B-35 | FRSYYI | EISPYTGYTY | GRLVTYSGAL | RASQSVSSAVA | SASSLYS | FDFTLA |
| 12B-37 | FSSYYI | YIYSYDRYTY | GGYYYVVRVM | RASQSVSSAVA | SASSLYS | SSSGLR |
| 12B-42 | FTRYII | SIDPSRGYTK | GLVYYYHYGL | RASQSVSSAVA | SASSLYS | LTLHLS |
| 12B-43 | FSRYAI | YIWPYTGTTI | VAHSSHVGQAM | RASQSVSSAVA | SASSLYS | GKTSPL |
| CD3-3 | FSKYVI | AISSSGGYTL | TIYVPGM | RASQSVSSAVA | SASSLYS | LDYSPW |
| CD3-8 | FPWYA | GISPGTGYTY | GGRYYAM | RASQSVSSAVA | SASSLYS | AWETPL |

### 6.13.21. Example 20: Bispecific B-Body Single Step Purification

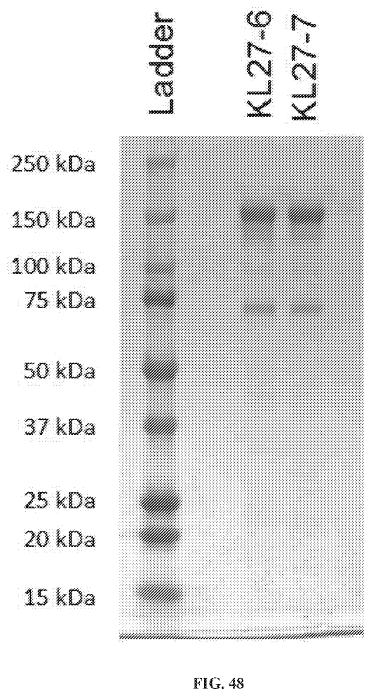

Anti-CH1 purification efficiency of bispecific antibodies was also tested for binding molecules having only standard knob-hole orthogonal mutations introduced into CH3 domains found in their native positions within the Fc portion of the bispecific antibody with no other domain modifications. Therefore, the two antibodies tested, KL27-6 and KL27-7, each contained two CH1 domains, one on each arm of the antibody. As described in more detail in Section 6.13.1, each bispecific antibody was expressed, purified from undesired protein products on an anti-CH1 column, and run on an SDS-PAGE gel. As shown in FIG. 48, a significant band at 75 kDa representing an incomplete bispecific antibody was present, interpreted as a complex containing only (i) a first and second or (ii) third and fourth polypeptide chains with reference to FIG. 3. Thus, methods using anti-CH1 to purify complete bispecific molecules that have a CH1 domain in each arm resulted in background contamination due to incomplete antibody complexes.

### 6.13.22. Example 21: Fc Mutations Reducing Effector Function

A series of engineered Fc variants were generated in the monoclonal IgG1 antibody trastuzumab (Herceptin, “WT-IgG1”) with mutations at positions L234, L235, and P329 of the CH2 domain. The specific mutations for the variants tested are described in Table 6 below and include sFc1 (PALALA), sFc7 (PGLALA), and sFc10 (PKLALA). All variants were produced by Expi293 expression as described herein.

TABLE 6
Fc variant analysis

| | | | | TM1 | TM2 | |
|---|---|---|---|---|---|---|
| Variant | L234 | L235 | P329 | (° C.) | (° C.) | FcγRIa Binding |
| sFc1 | A | A | P | 68.1 | 81.8 | Yes (weak) |
| sFc2 | G | G | P | 69.7 | 81.7 | No |
| sFc3 | L | L | A | 65.6 | 81.7 | Yes (strong) |
| sFc4 | A | A | A | 66.4 | 81.9 | No |
| sFc5 | G | G | A | 67.5 | 81.5 | No |
| sFc6 | L | L | G | 64.6 | 81.4 | Yes (strong) |
| sFc7 | A | A | G | 65.7 | 81.8 | No |
| sFc8 | G | G | G | 66.2 | 81.8 | No |
| sFc9 | L | L | K | 64.5 | 81.2 | Yes (weak) |
| sFc10 | A | A | K | 65.3 | 81.1 | No |
| sFc11 | G | G | K | 66.2 | 81.5 | No |
| wt IgG1 | L | L | P | 66.2 | 81.1 | Yes (strong) |

The protein melting temperature was determined using the Protein Thermal Shift Dye Kit (Thermo Fisher). Briefly, proteins of interest were brought to a concentration of 1 mg/ml. Thermal shift dye mix (water, Thermal shift buffer, and Thermal Shift Dye) was added to the protein of interest. The protein/thermal dye mix was added to glass capillary tubes and analyzed using a thermal gradient on a Roche Light Cycler. Proteins were incubated at 37° C. for 2 minutes before initiating a thermal gradient from 37° C. to 99° C. with a temperature increase rate of 0.1° C./sec. Fluorescence increase was measured over time and used to calculate the thermal melting temperature. Table 6 depicts results from the Protein Thermal Shift experiment above. All variants showed comparable stability as the wild-type IgG.

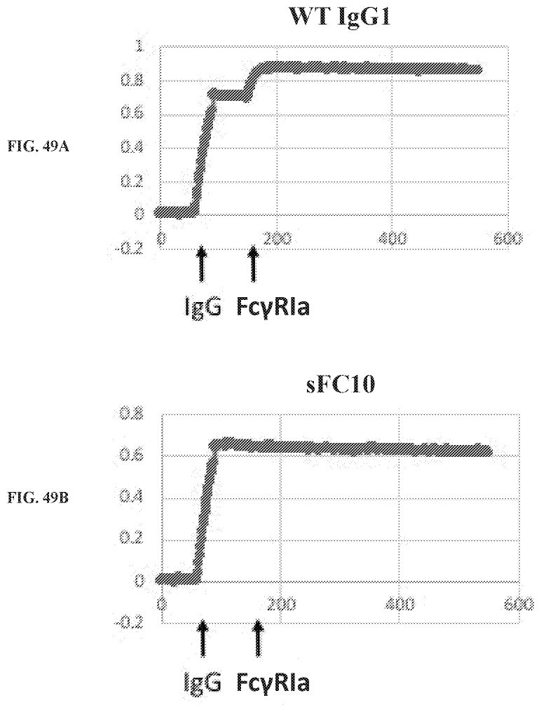

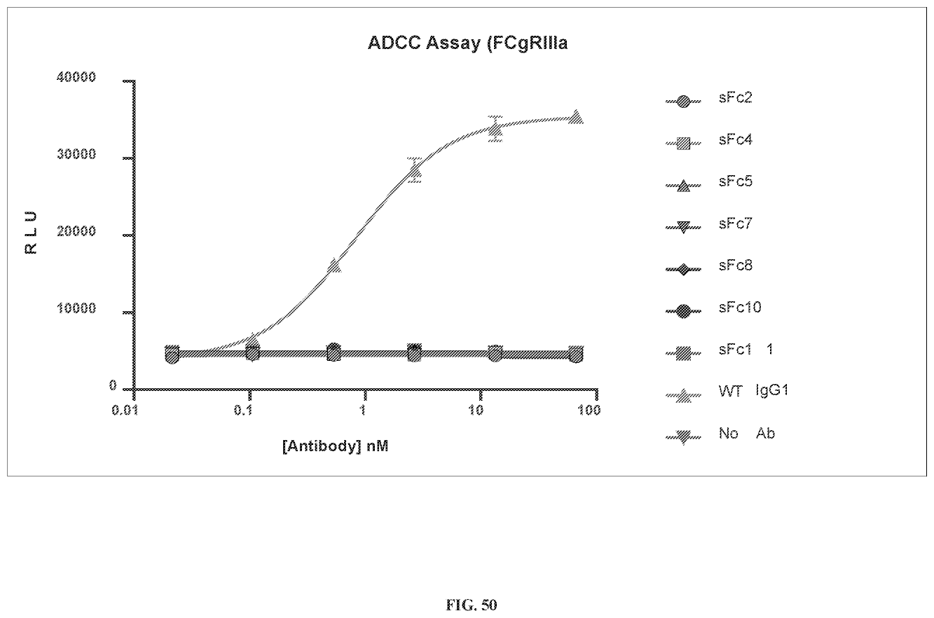

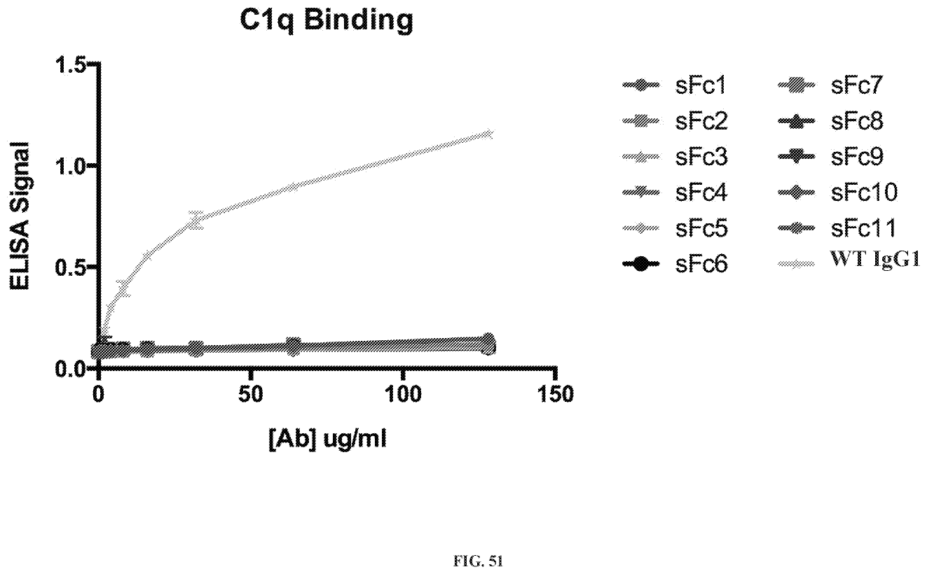

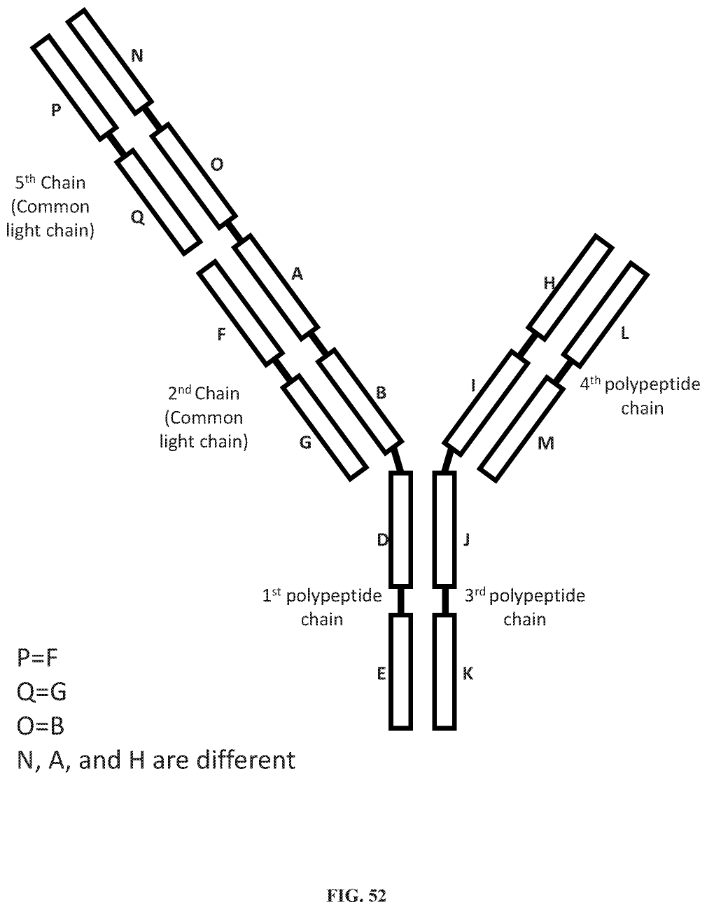

WT-IgG1 and the Fc variants were immobilized to the Octet biosensor and soluble FcγRIa was added to the system to determine binding. FIG. 49 shows Octet (Pall ForteBio) biolayer interferometry analysis demonstrating FcγRIa binding to trastuzumab (FIG. 49A “WT IgG1”), but not sFc10 (FIG. 49B). Upon addition of FcγRIa, an increase in signal was seen for trastuzumab, but no observable signal increase was detected for sFc10 demonstrating FcγRIa no longer binds the antibody with the engineered mutations. Binding summaries for the variants tested presented in Table 6. In addition, all variants retained strong binding to HER2 (not shown).

WT-IgG1 and the Fc variants were tested in an antibody dependent cellular cytotoxicity (ADCC) assay as another measure of FcγR binding. Briefly, the impact of selected Fc mutations on FCγRIIIa effector function was assessed using the ADCC Bioreporter Assay Kit (Promega). A serial dilution of each variant was incubated with SKBR3 cells. The reactions were then incubated at 37° C. in a humidified CO2 incubator with the ADCC Bioassay effector cells according to the manufacturer's protocol and incubated for 6 to 24 hrs. After incubation, the Bio-Glo™ Luciferase Assay Reagent was added to each sample and the luminescent signal was measured with a plate reader with glow-type luminescence read capabilities.

As shown in FIG. 50, trastuzumab (Herceptin, “WT-IgG1”) demonstrated killing, while none of the Fc variants tested resulted in detectable levels of killing.

WT-IgG1 and the Fc variants were also tested for complement component C1q binding by ELISA. Briefly, up to 128 μg/ml IgG was immobilized for each of the variants. The ELISA was performed with 12 μg/ml C1q and 1/400 dilution of the C1q-HRP secondary antibody. Washes and samples were diluted in PBST-BSA (1%).

As shown in FIG. 51, trastuzumab (Herceptin, “WT-IgG1”) demonstrated C1q binding, while neither sFc1, sFc7, nor sFc10 resulted in detectable C1q binding. Thus, the results demonstrate that the Fc variants tested have reduced levels of Fc effector function.

### 6.13.23. Example 22: Discovery of New ABSs with Common VL Sequences Using a Common Light Chain Library

Trivalent trispecific binding molecules are identified, de novo, that have two new antigen binding sites (ABSs) that share a common light chain variable sequence. The common light chain library used restricts the diversification of CDRs to the heavy chain variable domain (VH). Common light chain libraries are created for in vitro display (phage display, yeast display, mammalian display, etc) or in humanized animal models. Selections performed with common light chain libraries produce trivalent trispecific binding molecules with diversity in the VH domain for two ABSs but a single sequence in the light chain variable domain (VL) common to both ABSs.

### 6.13.23.1. Common Light Chain Phage Library Construction

The common light chain library is created using sequences derived from a specific heavy chain variable domain (e.g., VH3-23) and a specific light chain variable domain (e.g., Vk-1). Phage display libraries can be created through a number of strategies known in the art. Here, Fab-formatted phage libraries are constructed using expression vectors capable of replication and expression in phage (also referred to as a phagemid). Both the heavy chain and the light chain are encoded for in the same expression vector, where the heavy chain is fused to a truncated variant of the phage coat protein pIII. The light chain and heavy chain are expressed as a separate polypeptides, and the light chain and heavy chain-pIII fusion assemble in the bacterial periplasm, where the redox potential enables disulfide bond formation, to form the antibody containing the candidate ABS.

To construct common light chain libraries, a single light chain variable domain is chosen where the common light chain CDR1 (L1) and CDR2 (L2) remain the human germline sequence and the CDR3 (L3) is chosen from a consensus sequence that is able to support binding to a large variety of antigens. Libraries can also be constructed wherein all VL CDRs in the common light chain are varied to represent the full human diversity of light chain variable sequences. For a given common light chain, all CDR positions of the VH domain are diversified to match the positional amino acid frequency by CDR length found in the human antibody repertoire. Diversity can be created through a number of strategies known in the art. Here, Kunkel mutagenesis is performed with primers introducing diversity into VH CDRs H1, H2 and H3 to mimic the diversity found in the natural antibody repertoire, as described in more detail in Kunkel, TA (PNAS Jan. 1, 1985. 82 (2) 488-492), herein incorporated by reference in its entirety. Briefly, single-stranded DNA is prepared from isolated phage using standard procedures and Kunkel mutagenesis carried out. Chemically synthesized DNA is then electroporated into TG1 cells, followed by recovery. Recovered cells are sub-cultured and infected with M13K07 helper phage to produce the phage library.

### 6.13.23.2. Common Light Chain Phage Screening

Phage panning is performed using standard procedures. Briefly, the first round of phage panning is performed mixing target antigens immobilized on streptavidin magnetic beads with ˜5×10¹² phages from the prepared library described above in a volume of 1 mL in PBST-2% BSA. After a one-hour incubation, the bead-bound phage are separated from the supernatant using a magnetic stand. Beads are washed three times to remove non-specifically bound phage and then added to ER2738 cells (5 mL) at OD₆₀₀˜0.6. After 20 minutes, infected cells are sub-cultured in 25 mL 2×YT+Ampicillin and M13K07 helper phage and allowed to grow overnight at 37° C. with vigorous shaking. The next day, phage are prepared using standard procedures by PEG precipitation. Pre-clearance of phage specific to SAV-coated beads is performed prior to panning. The second round of panning is performed using the KingFisher magnetic bead handler with 100 nM bead-immobilized antigen using standard procedures. In total, 3-4 rounds of phage panning are performed to enrich in phage displaying Fabs specific for the target antigen. Target-specific enrichment is then confirmed using polyclonal and monoclonal phage ELISA.

### 6.13.23.3. Trivalent Trispecific Antibody Discovery Using Common Light Chain Libraries

A trivalent trispecific antibody having two new ABSs that share a common light chain variable region (VL), where each recognizes a different antigen or different epitope of the same antigen, is identified in a discovery campaign. The trivalent trispecific antibody also has a third ABS, which does not share the common VL region, which is specific for a third distinct antigen.

A phage display campaign using a common light chain library, described above, is used to separately identify candidate ABSs that bind Antigen 1 (A1) or Antigen 2 (A2). ABSs that share a common VL but with different VHs that impart specificity for Antigen 1 or Antigen 2 are identified, with affinities ranging from 1 μM to below 1 nM. ABSs are reformatted into full length human bivalent monospecific native IgG1 architecture for characterization. Candidates are evaluated for binding affinity, epitope, and generally biophysical qualities (expression, purity, developability, etc.). ABSs having individual binding affinities ranging from 10 nM-1 μM, or preferably 50 nM-250 nM, which bind both Antigen 1 and Antigen 2 are identified.

VL and VH domains from the parental IgG candidates for Antigen 1 and Antigen 2 are reformatted into the 1×2 antibody format below, along with the third ABS specific for Antigen 3 (A3). The combinations of candidates are expressed via transient mammalian expression, purified, and tested for the ability to simultaneously co-engage both antigens on the cell surface. Candidates have the following binding properties:
Monovalent KD to Antigen 1: 50 to 100 nM
Monovalent KD to Antigen 2: 50 to 100 nM
Monovalent KD to Antigen 3: <100 nM
Bivalent avidity to double-positive cell: <10 nM

Chain Architecture of the Candidate (with reference to FIG. 55):
Chain 1: VH_{A1}-CH3-CH2-CH3_{Hole}
Chain 2 and Chain 6: VL_{A1/A2-common}-CH3
Chain 3: VH_{A2}-CH3-VH_{A3}-CH1-CH2-CH3_{Knob}
Chain 4: VL_{A3}-CL1
(note: VL_{A3}-CL1 and VH_{A3}-CH1 can be swapped, and all domains may possess any of the orthogonal mutations previously described)

### 6.13.23.4. T Cell Redirecting Trivalent Trispecific Antibody Discovery Using Common Light Chain Libraries

A trivalent trispecific antibody having two new ABSs that share a common light chain variable region (VL), where each recognizes a different tumor antigen or different epitope of the same tumor antigen, is identified in a discovery campaign. The trivalent trispecific antibody also has a third ABS, which does not share the common VL region, which is specific for a T cell molecule useful for T cell redirection therapy, such as CD3 epsilon. This trivalent trispecific antibody can also be designed to utilize low monovalent affinities to the two tumor antigens yet achieve strong bivalent binding to tumor cells that present both antigens on the cell surface.

A phage display campaign using a common light chain library, described above, is used to separately identify candidate ABSs that bind Tumor Antigen 1 (TA1) or Tumor Antigen 2 (TA2). ABSs that share a common VL but with different VHs that impart specificity for Tumor Antigen 1 or Tumor Antigen 2 are identified, with affinities ranging from 1 μM to below 1 nM. ABSs are reformatted into full length human bivalent monospecific native IgG1 architecture for characterization. Candidates are evaluated for binding affinity, epitope, and generally biophysical qualities (expression, purity, developability, etc.). ABSs having individual binding affinities ranging from 10 nM-1 μM, or preferably 50 nM-250 nM, which bind both Antigen 1 and Antigen 2 are identified.

VL and VH domains from the parental IgG candidates for Tumor Antigen 1 and Tumor Antigen 2 are reformatted into the 1×2 antibody format below, along with a third known ABS specific for CD3 (e.g., SP34, OKT3, etc., and humanized variants thereof). The combinations of candidates are expressed via transient mammalian expression, purified, and tested for the ability to simultaneously co-engage both antigens on the cell surface. Additional functional assays, such as T cell killing and proliferation assays, are performed to characterize antibody efficacy. Candidates have the following binding properties:
Monovalent KD to Antigen 1: 50 to 100 nM
Monovalent KD to Antigen 2: 50 to 100 nM
Monovalent KD to CD3 epsilon: 20 to 100 nM
Bivalent avidity to double-positive tumor cell: <10 nM

Chain Architecture of the Candidate (with reference to FIG. 55):
Chain 1: VH_{TA1}-CH3-CH2-CH3_{Hole}
Chain 2 and Chain 6: VL_{TA1/TA2-common}-CH3
Chain 3: VH_{TA2}-CH3-VH_{CD3}-CH1-CH2-CH3_{Knob}
Chain 4: VL_{CD3}-CL1
(note: VL_{CD3}-CL1 and VH_{CD3}-CH1 can be swapped, and all domains may possess any of the orthogonal mutations previously described)

### 6.13.24. Example 23: Discovery of ABSs with Common VH Sequences Using a Common Heavy Chain Library

Trivalent trispecific binding molecules are identified, de novo, that have two new antigen binding sites (ABSs) that share a common heavy chain variable sequence. The common light chain library used restricts the diversification of CDRs to the light chain variable domain (VL). Common heavy chain libraries are created for in vitro display (phage display, yeast display, mammalian display, etc) or in humanized animal models. Selections performed with common heavy chain libraries produce trivalent trispecific binding molecules with diversity in the VL domain for two ABSs but a single sequence in the heavy chain variable domain (VH) common to both ABSs.

### 6.13.24.1. Common Heavy Chain Phage Library Construction

The common heavy chain library is created using sequences derived from a specific heavy chain variable domain (e.g., human VH3-23) and a specific light chain variable domain (e.g., human Vk-1). Phage display libraries can be created through a number of strategies known in the art. Here, Fab-formatted phage libraries are constructed using expression vectors capable of replication and expression in phage (also referred to as a phagemid). Both the heavy chain and the light chain are encoded for in the same expression vector, where the heavy chain is fused to a truncated variant of the phage coat protein pIII. The light chain and heavy chain are expressed as a separate polypeptides, and the light chain and heavy chain-pIII fusion assemble in the bacterial periplasm, where the redox potential enables disulfide bond formation, to form the antibody containing the candidate ABS.

To construct common heavy chain libraries, a single heavy chain variable domain is chosen where the common heavy chain CDR1 (H1) and CDR2 (H2) remain the human germline sequence and the CDR3 (H3) is chosen from a consensus sequence that is able to support binding to a large variety of antigens. Libraries can also be constructed wherein all VH CDRs in the common heavy chain are varied to represent the full human diversity of heavy chain variable sequences. For a given common heavy chain, all CDR positions of the VL domain are diversified to match the positional amino acid frequency by CDR length found in the human antibody repertoire. Diversity can be created through a number of strategies known in the art. Here, Kunkel mutagenesis is performed with primers introducing diversity into VL CDRs L1, L2 and L3 to mimic the diversity found in the natural antibody repertoire, as described in more detail in Kunkel, T A (PNAS Jan. 1, 1985. 82 (2) 488-492), herein incorporated by reference in its entirety. Briefly, single-stranded DNA is prepared from isolated phage using standard procedures and Kunkel mutagenesis carried out. Chemically synthesized DNA is then electroporated into TG1 cells, followed by recovery. Recovered cells are sub-cultured and infected with M13K07 helper phage to produce the phage library.

### 6.13.24.2. Common Heavy Chain Phage Screening

Phage panning is performed using standard procedures. Briefly, the first round of phage panning is performed mixing target antigens immobilized on streptavidin magnetic beads with ˜5×10¹² phages from the prepared library described above in a volume of 1 mL in PBST-2% BSA. After a one-hour incubation, the bead-bound phage are separated from the supernatant using a magnetic stand. Beads are washed three times to remove non-specifically bound phage and then added to ER2738 cells (5 mL) at OD₆₀₀˜0.6. After 20 minutes, infected cells are sub-cultured in 25 mL 2×YT+Ampicillin and M13K07 helper phage and allowed to grow overnight at 37° C. with vigorous shaking. The next day, phage are prepared using standard procedures by PEG precipitation. Pre-clearance of phage specific to SAV-coated beads is performed prior to panning. The second round of panning is performed using the KingFisher magnetic bead handler with 100 nM bead-immobilized antigen using standard procedures. In total, 3-4 rounds of phage panning are performed to enrich in phage displaying Fabs specific for the target antigen. Target-specific enrichment is then confirmed using polyclonal and monoclonal phage ELISA.

### 6.13.24.3. Trivalent Trispecific Antibody Discovery Using Common Heavy Chain Libraries

A trivalent trispecific antibody having two new ABSs that share a common heavy chain variable region (VH), where each recognizes a different antigen or different epitope of the same antigen, is identified in a discovery campaign. The trivalent trispecific antibody also has a third ABS, which does not share the common VH region, which is specific for a third distinct antigen.

A phage display campaign using a common heavy chain library, described above, is used to separately identify candidate ABSs that bind Antigen 1 (A1) or Antigen 2 (A2). ABSs that share a common VH but with different VLs that impart specificity for Antigen 1 or Antigen 2 are identified, with affinities ranging from 1 μM to below 1 nM. ABSs are reformatted into full length human bivalent monospecific native IgG1 architecture for characterization. Candidates are evaluated for binding affinity, epitope, and generally biophysical qualities (expression, purity, developability, etc.). ABSs having individual binding affinities ranging from 10 nM-1 μM, or preferably 50 nM-250 nM, which bind both Antigen 1 and Antigen 2 are identified.

VL and VH domains from the parental IgG candidates for Antigen 1 and Antigen 2 are reformatted into the 1×2 B-Body format below, along with the third ABS specific for Antigen 3 (A3). Exemplary 1×2 B-body scaffold chains are described by SEQ ID NOs: 78, 79, 81, and 82. The combinations of candidates are expressed via transient mammalian expression, purified, and tested for the ability to simultaneously co-engage both antigens on the cell surface. Candidates have the following binding properties:
Monovalent KD to Antigen 1: 50 to 100 nM
Monovalent KD to Antigen 2: 50 to 100 nM
Monovalent KD to Antigen 3: <100 nM
Bivalent avidity to double-positive cell: <10 nM

Chain Architecture of the Candidate (with reference to FIG. 55):
Chain 1: VL_{A1}-CH3-CH2-CH3_{Hole}
Chain 2 and Chain 6: VH_{A1/A2-common}-CH3
Chain 3: VL_{A2}-CH3-VL_{A3}-CL1-CH2-CH3_{Knob}
Chain 4: VH_{A3}-CH1
(note: VL_{A3}-CL1 and VH_{A3}-CH1 can be swapped, and all domains may possess any of the orthogonal mutations previously described)

### 6.13.24.4. T Cell Redirecting Trivalent Trispecific Antibody Discovery Using Common Heavy Chain Libraries

A trivalent trispecific antibody having two new ABSs that share a common heavy chain variable region (VH), where each recognizes a different tumor antigen or different epitope of the same tumor antigen, is identified in a discovery campaign. The trivalent trispecific antibody also has a third ABS, which does not share the common VH region, which is specific for a T cell molecule useful for T cell redirection therapy, such as CD3 epsilon. This trivalent trispecific antibody can also be designed to utilize low monovalent affinities to the two tumor antigens yet achieve strong bivalent binding to tumor cells that present both antigens on the cell surface.

A phage display campaign using a common heavy chain library, described above, is used to separately identify candidate ABSs that bind Tumor Antigen 1 (TA1) or Tumor Antigen 2 (TA2). ABSs that share a common VH but with different VLs that impart specificity for Tumor Antigen 1 or Tumor Antigen 2 are identified, with affinities ranging from 1 μM to below 1 nM. ABSs are reformatted into full length human bivalent monospecific native IgG1 architecture for characterization. Candidates are evaluated for binding affinity, epitope, and generally biophysical qualities (expression, purity, developability, etc.). ABSs having individual binding affinities ranging from 10 nM-1 μM, or preferably 50 nM-250 nM, which bind both Antigen 1 and Antigen 2 are identified.

VL and VH domains from the parental IgG candidates for Tumor Antigen 1 and Tumor Antigen 2 are reformatted into the 1×2 B-Body format below, along with the third ABS specific for CD3 (e.g., SP34, OKT3, etc., and humanized variants thereof). Exemplary 1×2 B-body scaffold chains are described by SEQ ID NOs: 78, 79, 81, and 82. The combinations of candidates are expressed via transient mammalian expression, purified, and tested for the ability to simultaneously co-engage both antigens on the cell surface. Additional functional assays, such as T cell killing and proliferation assays, are performed to characterize antibody efficacy. Candidates have the following binding properties:
Monovalent KD to Antigen 1: 50 to 100 nM
Monovalent KD to Antigen 2: 50 to 100 nM
Monovalent KD to CD3 epsilon: 20 to 100 nM
Bivalent avidity to double-positive tumor cell: <10 nM

Chain Architecture of the Candidate (with reference to FIG. 55):
Chain 1: VL_{TA1}-CH3-CH2-CH3_{Hole}
Chain 2 and Chain 6: VH_{TA1/TA2-common}-CH3
Chain 3: VL_{TA2}-CH3-VL_{CD3}-CL1-CH2-CH3_{Knob}
Chain 4: VH_{CD3}-CH1
(note: VL_{CD3}-CL1 and VH_{CD3}-CH1 can be swapped, and all domains may possess any of the orthogonal mutations previously described)

### 6.13.25. Example 24: Discovery of New ABSs with Common VL or VH Sequences Based on Known ABS Sequences

Trivalent trispecific binding molecules are identified, starting from a parent ABS sequence with known specificity, that have two new antigen binding sites (ABSs) that share either a common light chain variable sequence or a common heavy chain variable sequence. A common light chain library restricts the diversification of CDRs to the heavy chain variable domain (VH), while common heavy chain library restricts the diversification of CDRs to the heavy chain variable domain (VL). Common light or heavy chain libraries are created for in vitro display (phage display, yeast display, mammalian display, etc) or in humanized animal models.

While other libraries start de novo from VH or VL sequences, including germline sequences, that are agnostic regarding binding specificity, the screen performed here starts from an ABS with a known specificity that can include CDR sequences other than germline sequences. Starting from the known ABS sequences, the parent VH or VL sequence is paired, respectively, with either VL or VH sequences having diversity introduced into their CDRs, as previously described. Non-cognate VH/VL pairs (i.e., VH and VL pairs not from the parent ABS) are screened for binding to two antigens, Antigen 1 and Antigen 2, as previously described. One of the antigens can be the same antigen bound by the parent ABS. Additional reformatting of the VH and VL sequences to further characterize the candidate ABSs is performed, as previously described.

The screening and characterization results in trivalent trispecific binding molecules with two new ABSs, each specific for a different antigen or epitope, that share a common VL region or VH region sequence. The trivalent trispecific antibody also has a third ABS, which does not share the common VL or VH region, that is specific for a third distinct antigen.

### 6.13.25.1. Trivalent Trispecific Antibody Discovery Using Common Heavy Chains from Known ABSs

New antigen binding sites specific for the antigen human OX40 were determined starting from parent ABS sequences with known specificity for human OX40.

Briefly, a VH domain isolated from a phage panning campaign against human OX40 was used as a common heavy chain variable domain sequence and paired with the VL domains of 39 other ABS candidates, as well as its parent cognate VL domain, isolated from an OX40 campaign. In the campaign, the diversity of the VL domains was limited to the CDR3 sequence, keeping CDR1 and CDR2 constant. The VL CDR3 sequences of the candidates are presented in Table 7. The VH of OX40-13 was initially chosen for its relative lack of bulky residues in positions L92-L94 (CDRH1: GFTFSSYIIHW; CDRH2: WVAYIFPYSGETYYADS; CDRH3: CARGAYYYTDLVFDYW). ABS candidates were expressed in small-scale as monoclonal antibodies in Expi293 cells in a volume of 2 mL. After 5 days of expression, cleared supernatants were diluted 3-fold in PBST-BSA and tested for retained binding to biotinylated human OX40 via biolayer interferometry (Octet). Here, streptavidin sensors were immobilized with biotinylated OX40 until a binding response of ˜0.8 nm was reached. After establishing the baseline, the diluted supernatants were assessed for antigen binding.

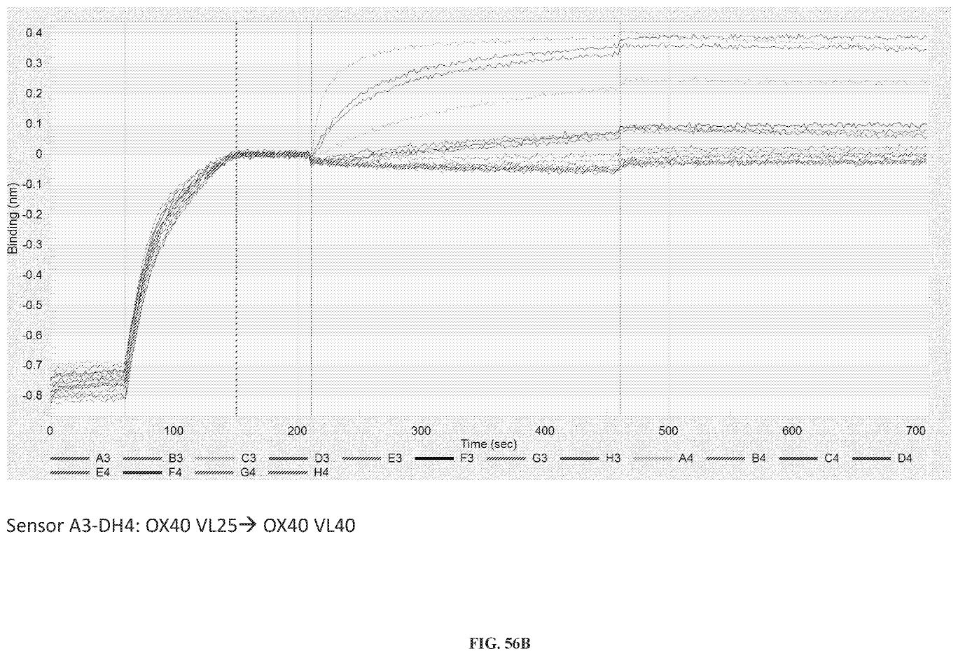

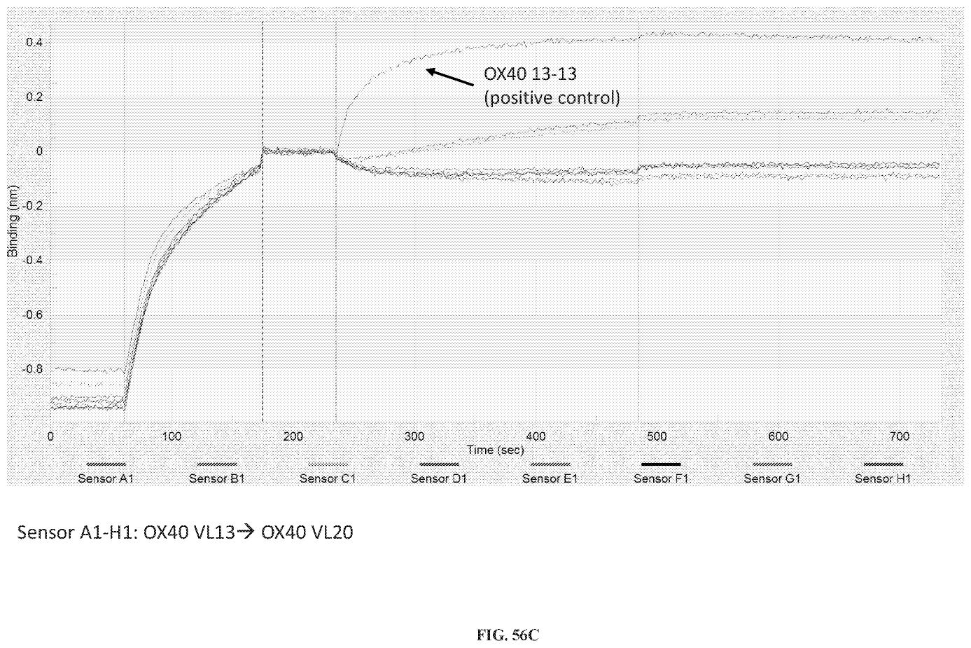

FIG. 56 shows the Octet binding analysis for the VL domains paired with the OX40-13 VH domain, with non-cognate VL domains 1-12 and 21-24 shown in FIG. 56A, non-cognate VL domains 25-40 shown in FIG. 56B, and non-cognate VL domains 14-20 and cognate VL domain VL13 shown in FIG. 56C. Several of the non-cognate VL domains demonstrated binding comparable to parent OX40-13 ABS, including the VL of OX40-1 and OX40-27. Others did not demonstrate detectable binding comparable to parent OX40-13 ABS. While still others demonstrated a range of intermediate binding between the parent OX40-13 ABS and the limit of detectable binding. Interestingly, several of the sequences, such as OX40-1 and OX40-27, diverge noticeably from the parent OX40-13 VL sequence suggesting many VL sequences may retain antigen recognition of the parent VH.

Two of the new OX40 ABSs discovered here, each paired with the common heavy chain variable sequence from OX40-13, are formatted into a trivalent trispecific antibody where the new ABS candidates do not cause significant loss in expression, yield, or binding properties.

TABLE 7
Candidate VLSequences

| Candidate VL | CDR3 |
|---|---|
| VL1 | QQFQDSPVTF |
| VL2 | QQYIYGPLTF |
| VL3 | QQYIYSPATF |
| VL4 | QQWYSDPETF |
| VL5 | QQYIYDPSTF |
| VL6 | QQYARPPRTF |
| VL7 | QQYYFWPWTF |
| VL8 | QQYVSSPETF |
| VL9 | QQYDYSPATF |
| VL10 | QQVDSTPVTF |
| VL11 | QQYTSHPGTF |
| VL12 | QQFYSSPETF |
| VL13 | QQYSSSPVTF |
| VL14 | QQWSAKLYTF |
| VL15 | QQYTSSPYTF |
| VL16 | QQADYSLTTF |
| VL17 | QQASWGLTTF |
| VL18 | QQYERIPYTF |
| VL19 | QQYYGSLYTF |
| VL20 | QQVTYTPLTF |
| VL21 | QQYYTSPETF |
| VL22 | QQLSSWPLTF |
| VL23 | QQSDSSPWTF |
| VL24 | QQWDDSPYTF |
| VL25 | QQLFSHPYTF |
| VL26 | QQWYSTPYTF |
| VL27 | QQAYGDLRTF |
| VL28 | QQGYSDPQTF |
| VL29 | QQGSSSPLTF |
| VL30 | QQYSDWPYTF |
| VL31 | QQHSSSLETF |
| VL32 | QQVDTSLGTF |
| VL33 | QQADTQPLTF |
| VL34 | QQWSSSPETF |
| VL35 | QQYHTSLHTF |
| VL36 | QQYYGGLPTF |
| VL37 | QQSYSSPYTF |
| VL38 | QQYYSEPVTF |
| VL39 | QQVHSYPSTF |
| VL40 | QQWTRSLTTF |

### 6.13.26. Example 25: Discovery of New ABS with Common VL or VH Sequence from Known ABS

Trivalent trispecific binding molecules are identified, starting from a parent ABS sequence with known specificity, which have a new antigen binding site (ABS) that shares either a common light chain variable sequence or a common heavy chain variable sequence with the parent ABS. A common light chain library restricts the diversification of CDRs to the heavy chain variable domain (VH), while common heavy chain library restricts the diversification of CDRs to the heavy chain variable domain (VL). Common light or heavy chain libraries are created for in vitro display (phage display, yeast display, mammalian display, etc) or in humanized animal models.

Other libraries start de novo from VH or VL sequences, including germline sequences, which are agnostic regarding binding specificity. However, the screen performed here starts from a parent ABS with a known specificity to discover new ABSs having a different antigen specificity while sharing a common VH or VL sequence with the parent ABS. Starting from the known ABS sequences, the parent VH or VL sequence is paired, respectively, with either VL or VH sequences having diversity introduced into their CDRs, as previously described. The pairs are screened for binding to an antigen of interest, as previously described. Additional reformatting of the VH and VL sequences to further characterize the candidate ABSs is performed, as previously described.

The screening and characterization results in trivalent trispecific binding molecules with a known parent ABS specific for a known antigen and a new ABS specific for a different antigen, where the ABSs share a common VL region or VH region sequence. The trivalent trispecific antibody also has a third ABS, which does not share the common VL or VH region, that is specific for a third distinct antigen.

### 6.13.26.1. Discovery of New ABS with Common VL Shared with Trastuzumab

A phage display campaign using a common light chain library is created using the light chain VL sequence of Trastuzumab specific for HER2. Human VH3-23 CDR sequences are diversified to match the positional amino acid frequency by CDR length found the in the human antibody repertoire and phage expressing paired VL/VH sequences are screened for binding to an antigen of interest (A2), as described previously. ABSs are reformatted into full length human bivalent monospecific native IgG1 architecture for characterization. Candidates are evaluated for binding affinity, epitope, and generally biophysical qualities (expression, purity, developability, etc.). ABSs having individual binding affinities ranging from 10 nM-1 μM, or preferably 50 nM-250 nM, which bind both Antigen 1 and Antigen 2 are identified.

VL and VH domains from the parental IgG candidates for the antigen of interest are reformatted into the 1×2 antibody format below, along with the third ABS specific for CD3 (e.g., SP34, OKT3, etc., and humanized variants thereof). The combinations of candidates are expressed via transient mammalian expression, purified, and tested for the ability to simultaneously co-engage both antigens on the cell surface. Additional functional assays, such as T cell killing and proliferation assays, are performed to characterize antibody efficacy. Candidates have the following binding properties:
Monovalent KD to Trastuzumab 1: 7 nM
Monovalent KD to Antigen of Interest (A2): 50 to 100 nM
Monovalent KD to CD3 epsilon: 20 to 100 nM
Bivalent avidity to double-positive tumor cell: <10 nM

Example Chain Architecture of the Candidate:
Chain 1: VH_{trastuzumab}-CH3-CH2-CH3_{Hole}
Chain 2 and Chain 6: VL_{trastuzumab}-CH3
Chain 3: VH_{A2}-CH3-VH_{CD3}-CH1-CH2-CH3_{Knob}
Chain 4: VL_{CD3}-CL1
(note: VL_{CD3}-CL1 and VH_{CD3}-CH1 can be swapped, and all domains may possess any of the orthogonal mutations previously described)

6.14. Sequences

>Example 1, bivalent monospecific construct CHAIN 1 [SEQ ID NO: 1]
(VL)~VEIKRTPREPQVYTLPPCRDELTKNQVSLWCLVKGFYPSDIAVEWESNGQPENNYKTTPPVLDSDGSFFLYSKL
TVDKSRWQQGNVFSCSVMHEALHNHYTQKSLSLSPGKDKTHTCPPCPAPELLGGPSVFLFPPKPKDTLMISRTPEVTCV
VVDVSHEDPEVKFNWYVDGVEVHNAKTKPREEQYNSTYRVVSVLTVLHQDWLNGKEYKCKVSNKALPAPIEKTISKAKG
QPREPQVYTLPPCRDELTKNQVSLWCLVKGFYPSDIAVEWESNGQPENNYKTTPPVLDSDGSFFLYSKLTVDKSRWQQG
NVFSCSVMHEALHNHYTQKSLSLSPGK
>Example 1, bivalent monospecific construct CHAIN 2 [SEQ ID NO: 2]
(VH)~VTVSSASPREPQVYTLPPCRDELTKNQVSLWCLVKGFYPSDIAVEWESNGQPENNYKTTPPVLDSDGSFFLYSK
LTVDKSRWQQGNVFSCSVMHEALHNHYTQKSLSLSPGK
>Example 1, bivalent, bispecific construct CHAIN 1 [SEQ ID NO: 3]
(VL)~VEIKRTPREPQVYTLPPCRDELTKNQVSLWCLVKGFYPSDIAVEWESNGQPENNYKTTPPVLDSDGSFFLYSKL
TVDKSRWQQGNVFSCSVMHEALHNHYTQKSLSLSPGKDKTHTCPPCPAPELLGGPSVFLFPPKPKDTLMISRTPEVTCV
VVDVSHEDPEVKFNWYVDGVEVHNAKTKPREEQYNSTYRVVSVLTVLHQDWLNGKEYKCKVSNKALPAPIEKTISKAKG
QPREPQVYTLPPCRDELTKIVQVSLWCLVKGFYPSDIAVEWESNGQPENNYKTTPPVLDSDGSFFLYSKLTVDKSRWQQ
GNVFSCSVMHEALHNHYTQKSLSLSPGK
VL- CH3- Hinge- CH2- CH3(knob)
>Example 1, bivalent, bispecific construct CHAIN 2 [SEQ ID NO: 4]
(VH)~VTVSSASPREPQVYTLPPCRDELTKNQVSLWCLVKGFYPSDIAVEWESNGQPENNYKTTPPVLDSDGSFFLYSK
LTVDKSRWQQGNVFSCSVMHEALHNHYTQKSLSLSPGK
VH- CH3
>Example 1, bivalent, bispecific construct CHAIN 3_[SEQ ID NO: 5]
(VL)~VEIKRTVAAPSVFIFPPSDEQLKSGTASVVCLLNNFYPREAKVQWKVDNALQSGNSQESVTEQDSKDSTYSLSS
TLTLSKADYEKHKVYACEVTHQGLSSPVTKSFNRGECDKTHTCPPCPAPELLGGPSVFLFPPKPKDTLMISRTPEVTCV
VVDVSHEDPEVKFNWYVDGVEVHNAKTKPREEQYNSTYRVVSVLTVLHQDWLNGKEYKCKVSNKALPAPIEKTISKAKG
QPREPQVCTLPPSREEMTKNQVSLSCAVKGFYPSDIAVEWESNGQPENNYKTTPPVLDSDGSFFLVSKLTVDKSRWQQG
NVFSCSVMHEALHNHYTQKSLSLSPGK
VL- CL- Hinge- CH2- CH3(hole)
>Example 1, bivalent, bispecific construct CHAIN4 [SEQ ID NO: 6]
(VH)~VTVSSASTKGPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPPKSC
VH- CH1
>Fc Fragment of Human IgG1 [SEQ ID NO: 7]
GQPREPQVYTLPPSRDELTKNQVSLTCLVKGFYPSDIAVEWESNGQPENNYKTTPPVLDSDGSFFLYSKLTVDKSRWQQ
GNVFSCSVMHEALHNHYTQKSLSLSP
>BC1 chain 1 [SEQ ID NO: 8]
DIQMTQSPSSLSASVGDRVTITCRASQDVNTAVAWYQQKPGKAPKLLIYSASFLYSGVPSRFSGSRSGTDFTLTISSLQ
PEDFATYYCQQHYTTPPTFGQGTKVEIKRTPREPQVYTLPPSRDELTKNQVSLKCLVKGFYPSDIAVEWESNGQPENNY
KTTPPVLDSDGSFFLYSKLTVDKSRWQQGNVFSCSVMHEALHNHYTQKSLSLSKSCDKTHTCPPCPAPELLGGPSVFLF
PPKPKDTLMISRTPEVTCVVVDVSHEDPEVKFNWYVDGVEVHNAKTKPREEQYNSTYRVVSVLTVLHQDWLNGKEYKCK
VSNKALPAPIEKTISKAKGQPREPQVYTLPPCRDELTKNQVSLWCLVKGFYPSDIAVEWESNGQPENNYKTTPPVLDSD
GSFFLYSKLTVDKSRWQQGNVFSCSVMHEALHNHYTQKSLSLSPGK
Domain arrangement:
A- B- Hinge- D- E
VL- CH3- Hinge- CH2- CH3(knob,)
Mutations in first CH3 (Domain B):
T366K; 445K, 446S, 447C insertion
Mutations in second CH3 (Domain E):
S354C, T366W
>BC1 chain 2 [SEQ ID NO: 9]
EVQLVESGGGLVQPGGSLRLSCAASGFTFSDYDIHWVRQAPGKGLEWVGDITPYDGTTNYADSVKGRFTISADTSKNTA
YLQMNSLRAEDTAVYYCARLVGEIATGFDYWGQGTLVTVSSASPREPQVYTDPPSRDELTKNQVSLTCLVKGFYPSDIA
VEWESNGQPENNYKTTPPVLDSDGSFFLYSKLTVDKSRWQQGNVFSCSVMHEALHNHYTQKSLSLSGEC
Domain arrangement:
F- G
VH- CH3
Mutations in CH3 (Domain G):
L351D; 445G, 446E, 447C insertion
>BC1 chain 3 [SEQ ID NO: 10]
EIVLTQSPATLSLSPGERATLSCRASQSVSSYLAWYQQKPGQAPRLLIYDASNRATGIPARFSGSGSGTDFTLTISSLE
PEDFAVYYCQQSSNWPRTFGQGTKVEIKRTVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKSFNRGECDKTHTCPPCPAPELLGGPSVFLF
PPKPKDTLMISRTPEVTCVVVDVSHEDPEVKFNWYVDGVEVHNAKTKPREEQYNSTYRVVSVLTVLHQDWLNGKEYKCK
VSNKALPAPIEKTISKAKGQPREPQVCTLPPSREEMTKNQVSLSCAVKGFYPSDIAVEWESNGQPENNYKTTPPVLDSD
GSFFLVSKLTVDKSRWQQGNFFSCSVMHEALHNHYTQKSLSLSPGK
Domain arrangement:
H- I- Hinge- J- K
VL- CL- Hinge- CH2- CH3(hole,)
Mutations in CH3 (domain K):
Y349C, D356E, L358M, T366S, L368A, Y407V
>BC1 chain 4 [SEQ ID NO: 11]
QVQLVESGGGVVQPGRSLRLDCKASGITFSNSGMHWVRQAPGKGLEWVAVIWYDGSKRYYADSVKGRFTISRDNSKNTL
FLQMNSLRAEDTAVYYCATNDDYWGQGTLVTVSSASTKGPSVFPLAPSSKSTSGGTAALGCLVKDYFPEPVTVSWNSGA
LTSGVHTFPAVLQSSGLYSLSSVVTVPSSSLGTQTYICNVNHKPSNTKVDKKVEPPKSC
Domain arrangement:
L- M
VH- CH1
>BC1 Domain A [SEQ ID NO: 12]
DIQMTQSPSSLSASVGDRVTITCRASQDVNTAVAWYQQKPGKAPKLLIYSASFLYSGVPSRFSGSRSGTDFTLTISSLQ
PEDFATYYCQQHYTTPPTFGQGTKVEIKRT
>BC1 Domain B [SEQ ID NO: 13]
PREPQVYTLPPSRDELTKNQVSLKCLVKGFYPSDIAVEWESNGQPENNYKTTPPVLDSDGSFFLYSKLTVDKSRWQQGN
VFSCSVMHEALHNHYTQKSLSLSKSC
>BC1 Domain D [SEQ ID NO: 14]
APELLGGPSVFLFPPKPKDTLMISRTPEVTCVVVDVSHEDPEVKFNWYVDGVEVHNAKTKPREEQYNSTYRVVSVLTVL
HQDWLNGKEYKCKVSNKALPAPIEKTISKAK
>BC1 Domain E [SEQ ID NO: 15]
GQPREPQVYTLPPCRDELTKNQVSLWCLVKGFYPSDIAVEWESNGQPENNYKTTPPVLDSDGSFFLYSKLTVDKSRWQQ
GNVFSCSVMHEALHNHYTQKSLSLSPGK
>BC1 Domain F [SEQ ID NO: 16]
EVQLVESGGGLVQPGGSLRLSCAASGFTFSDYDIHWVRQAPGKGLEWVGDITPYDGTTNYADSVKGRFTISADTSKNTA
YLQMNSLRAEDTAVYYCARLVGEIATGFDYWGQGTLVTVSSAS
>BC1 Domain G [SEQ ID NO: 17]
PREPQVYTDPPSRDELTKNQVSLTCLVKGFYPSDIAVEWESNGQPENNYKTTPPVLDSDGSFFLYSKLTVDKSRWQQGN
VFSCSVMHEALHNHYTQKSLSLSGEC
>BC1 Domain H [SEQ ID NO: 18]
EIVLTQSPATLSLSPGERATLSCRASQSVSSYLAWYQQKPGQAPRLLIYDASNRATGIPARFSGSGSGTDFTLTISSLE
PEDFAVYYCQQSSNWPRTFGQGTKVEIK
>BC1 Domain I [SEQ ID NO: 19]
RTVAAPSVFIFPPSDEQLKSGTASVVCLLNNFYPREAKVQWKVDNALQSGNSQESVTEQDSKDSTYSLSSTLTLSKADY
EKHKVYACEVTHQGLSSPVTKSFNRGEC
>BC1 Domain J [SEQ ID NO: 20]
APELLGGPSVFLFPPKPKDTLMISRTPEVTCVVVDVSHEDPEVKFNWYVDGVEVHNAKTKPREEQYNSTYRVVSVLTVL
HQDWLNGKEYKCKVSNKALPAPIEKTISKAK
>BC1 Domain K [SEQ ID NO: 21]
GQPREPQVCTLPPSREEMTKNQVSLSCAVKGFYPSDIAVEWESNGQPENNYKTTPPVLDSDGSFFLVSKLTVDKSRWQQ
GNVFSCSVMHEALHNHYTQKSLSLSPGK
>BC1 Domain L [SEQ ID NO: 22]
QVQLVESGGGVVQPGRSLRLDCKASGITFSNSGMHWVRQAPGKGLEWVAVIWYDGSKRYYADSVKGRFTISRDNSKNTL
FLQMNSLRAEDTAVYYCATNDDYWGQGTLVTVSS
>BC1 Domain M [SEQ ID NO: 23]
ASTKGPSVFPLAPSSKSTSGGTAALGCLVKDYFPEPVTVSWNSGALTSGVHTFPAVLQSSGLYSLSSVVTVPSSSLGTQ
TYICNVNHKPSNTKVDKKVEPPKSC
>BC28 chain 1 [SEQ ID NO: 24]
DIQMTQSPSSLSASVGDRVTITCRASQDVNTAVAWYQQKPGKAPKLLIYSASFLYSGVPSRFSGSRSGTDFTLTISSLQ
PEDFATYYCQQHYTTPPTFGQGTKVEIKRTPREPQVCTLPPSRDELTKNQVSLTCLVKGFYPSDIAVEWESNGQPENNY
KTTPPVLDSDGSFFLYSKLTVDKSRWQQGNVFSCSVMHEALHNHYTQKSLSLSPGKDKTHTCPPCPAPELLGGPSVFLF
PPKPKDTLMISRTPEVTCVVVDVSHEDPEVKFNWYVDGVEVHNAKTKPREEQYNSTYRVVSVLTVLHQDWLNGKEYKCK
VSNKALPAPIEKTISKAKGQPREPQVYTLPPCRDELTKNQVSLWCLVKGFYPSDIAVEWESNGQPENNYKTTPPVLDSD
GSFFLYSKLTVDKSRWQQGNVFSCSVMHEALHNHYTQKSLSLSPGK
Domain arrangement:
A- B- Hinge- D- E
VL- CH3- Hinge- CH2- CH3(knob)
Mutations in domain B:
Y349C; 445P, 446G, 447K insertion
Mutations in domain E:
S354C, T366W
>BC28 chain 2 [SEQ ID NO: 25]
EVQLVESGGGLVQPGGSLRLSCAASGFTFSDYDIHWVRQAPGKGLEWVGDITPYDGTTNYADSVKGRFTISADTSKNTA
YLQMNSLRAEDTAVYYCARLVGEIATGFDYWGQGTLVTVSSASPREPQVYTLPPCRDELTKNQVSLTCLVKGFYPSDIA
VEWESNGQPENNYKTTPPVLDSDGSFFLYSKLTVDKSRWQQGNVFSCSVMHEALHNHYTQKSLSLSPGK
Domain arrangement:
F- G
VH- CH3
Mutations in domain G:
S354C; 445P, 446G, 447K insertion
>BC28 domain A [SEQ ID NO: 26]
DIQMTQSPSSLSASVGDRVTITCRASQDVNTAVAWYQQKPGKAPKLLIYSASFLYSGVPSRFSGSRSGTDFTLTISSLQ
PEDFATYYCQQHYTTPPTFGQGTKVEIKRT
>BC28 domain B [SEQ ID NO: 27]
PREPQVCTLPPSRDELTKNQVSLTCLVKGFYPSDIAVEWESNGQPENNYKTTPPVLDSDGSFFLYSKLTVDKSRWQQGN
VFSCSVMHEALHNHYTQKSLSLSPGK
>BC28 domain D [SEQ ID NO: 28]
APELLGGPSVFLFPPKPKDTLMISRTPEVTCVVVDVSHEDPEVKFNWYVDGVEVHNAKTKPREEQYNSTYRVVSVLTVL
HQDWLNGKEYKCKVSNKALPAPIEKTISKAK
>BC28 domain E [SEQ ID NO: 29]
GQPREPQVYTLPPCRDELTKNQVSLWCLVKGFYPSDIAVEWESNGQPENNYKTTPPVLDSDGSFFLYSKLTVDKSRWQQ
GNVFSCSVMHEALHNHYTQKSLSLSPGK
>BC28 domain F [SEQ ID NO: 30]
EVQLVESGGGLVQPGGSLRLSCAASGFTFSDYDIHWVRQAPGKGLEWVGDITPYDGTTNYADSVKGRFTISADTSKNTA
YLQMNSLRAEDTAVYYCARLVGEIATGFDYWGQGTLVTVSSAS
>BC28 domain G [SEQ ID NO: 31]
PREPQVYTLPPCRDELTKNQVSLTCLVKGFYPSDIAVEWESNGQPENNYKTTPPVLDSDGSFFLYSKLTVDKSRWQQGN
VFSCSVMHEALHNHYTQKSLSLSPGK
>BC44 chain 1 [SEQ ID NO: 32]
DIQMTQSPSSLSASVGDRVTITCRASQDVNTAVAWYQQKPGKAPKLLIYSASFLYSGVPSRFSGSRSGTDFTLTISSLQ
PEDFATYYCQQHYTTPPTFGQGTKVEIKRTVREPQVCTLPPSRDELTKNQVSLTCLVKGFYPSDIAVEWESNGQPENNY
KTTPPVLDSDGSFFLYSKLTVDKSRWQQGNVFSCSVMHEALHNHYTQKSLSLSPGKDKTHTCPPCPAPELLGGPSVFLF
PPKPKDTLMISRTPEVTCVVVDVSHEDPEVKFNWYVDGVEVHNAKTKPREEQYNSTYRVVSVLTVLHQDWLNGKEYKCK
VSNKALPAPIEKTISKAKGQPREPQVYTLPPCRDELTKNQVSLWCLVKGFYPSDIAVEWESNGQPENNYKTTPPVLDSD
GSFFLYSKLTVDKSRWQQGNVFSCSVMHEALHNHYTQKSLSLSPGK
Domain arrangement:
A- B- Hinge- D- E
VL- CH3- Hinge- CH2- CH3(knob)
Mutations in domain B:
P343V, Y349C; 445P, 446G, 447K insertion
Mutations in domain E:
S354C, T366W
>BC44 Domain A [SEQ ID NO: 33]
DIQMTQSPSSLSASVGDRVTITCRASQDVNTAVAWYQQKPGKAPKLLIYSASFLYSGVPSRFSGSRSGTDFTLTISSLQ
PEDFATYYCQQHYTTPPTFGQGTKVEIKRT
>BC44 Domain B [SEQ ID NO: 34]
VREPQVCTLPPSRDELTKNQVSLTCLVKGFYPSDIAVEWESNGQPENNYKTTPPVLDSDGSFFLYSKLTVDKSRWQQ
GNVFSCSVMHEALHNHYTQKSLSLSPGK
>BC44 Domain D [SEQ ID NO: 35]
APELLGGPSVFLFPPKPKDTLMISRTPEVTCVVVDVSHEDPEVKFNWYVDGVEVHNAKTKPREEQYNSTYRVVSVLTVL
HQDWLNGKEYKCKVSNKALPAPIEKTISKAK
>BC44 Domain E [SEQ ID NO: 36]
GQPREPQVYTLPPCRDELTKNQVSLWCLVKGFYPSDIAVEWESNGQPENNYKTTPPVLDSDGSFFLYSKLTVDKSRWQQ
GNVFSCSVMHEALHNHYTQKSLSLSPGK
>BC28 bivalent chain 3 equivalent to SEQ ID NO: 10
>BC28 bivalent chain 4 equivalent to SEQ ID NO: 11
>BC28 1x2 chain 3 [SEQ ID NO: 37]
DIQMTQSPSSLSASVGDRVTITCRASQDVNTAVAWYQQKPGKAPKLLIYSASFLYSGVPSRFSGSRSGTDFTLTISSLQ
PEDFATYYCQQHYTTPPTFGQGTKVEIKRTPREPQVCTLPPSRDELTKNQVSLTCLVKGFYPSDIAVEWESNGQPENNY
KTTPPVLDSDGSFFLYSKLTVDKSRWQQGNVFSCSVMHEALHNHYTQKSLSLSPGKGSGSGS

[symbol]

[symbol]

[symbol]

[symbol]

[symbol]

RTVAAPSVFIFPPSDEQLKSGTASVVCLLNNFYPREAKVQWKV
DNALQSGNSQESVTEQDSKDSTYSLSSTLTLSKADYEKHKVYACEVTHQGLSSPVTKSFNRGECDKTHTCPPCPAPELL
GGPSVFLFPPKPKDTLMISRTPEVTCVVVDVSHEDPEVKFNWYVGVEVHNAKTKPREEQYNSTYRVVSVLTVLHQDWLN
GKEYKCKVSNKALPAPIEKTISKAKGQPREPQVCTLPPSREEMTKNQVSLSCAVKGFYPSDIAVEWESNGQPENNYKTT
PPVLDSGSFFLVSKLTVDKSRWQQGNVFSCSVMHEALHNHYTQKSLSLSPGK
Domain arrangement:
R- S- linker- H- I- Hinge- J- K-
VL- CH3- linker- VL- CL- Hinge- CH2- CH3(hole)
Mutations in domain S:
Y349C; 445P, 446G, 447K insertion
Six amino acids linker insertion: GSGSGS
Mutations in domain K:
Y349C, D356E, L358M, T366S, L368A, Y407V
>BC28 1x2 domain R [SEQ ID NO: 38]
DIQMTQSPSSLSASVGDRVTITCRASQDVNTAVAWYQQKPGKAPKLLIYSASFLYSGVPSRFSGSRSGTDFTLTISSLQ
PEDFATYYCQQHYTTPPTEGQGTKVEIKRT
>BC28 1x2 domain S [SEQ ID NO: 39]
PREPQVCTLPPSRDELTKNQVSLTCLVKGFYPSDIAVEWESNGQPENNYKTTPPVLDSDGSFFLYSKLTVDKSRWQQGN
VFSCSVMHEALHNHYTQKSLSLSPGK
>BC28 1x2 linker [SEQ ID NO: 40]
GSGSGS
>BC28 1x2 domain H [SEQ ID NO: 41]
EIVLTQSPATLSLSPGERATLSCRASQSVSSYLAWYQQKPGQAPRLLIYDASNRATGIPARFSGSGSGTDFTLTISSLE
PEDFAVYYCQQSSNWPRTFGQGTKVEIK
>BC28 1x2 domain I [SEQ ID NO: 42]
RTVAAPSVFIFPPSDEQLKSGTASVVCLLNNFYPREAKVQWKVDNALQSGNSQESVTEQDSKDSTYSLSSTLTLSKADY
EKHKVYACEVTHQGLSSPVTKSFNRGEC
>BC28 1x2 domain J [SEQ ID NO: 43]
APELLGGPSVFLEPPKPKDTLMISRTPEVTCVVVDVSHEDPEVKFNWYVDGVEVHNAKTKPREEQYNSTYRVVSVLTVL
HQDWLNGKEYKCKVSNKALPAPIEKTISKAK
>BC28 1x2 domain K [SEQ ID NO: 44]
GQPREPQVCTLPPSREEMTKNQVSLSCAVKGFYPSDIAVEWESNGQPENNYKTTPPVLDSDGSFFLVSKLTVDKSRWQQ
GNVFSCSVMHEALHNHYTQKSLSLSPGK
>BC28-1x1xla chain 3 [SEQ ID NO: 45]
DIQMTQSPSSLSASVGDRVTITCRASQSVSSAVAWYQQKPGKAPKLLIYSASSLYSGVPSRFSGSRSGTDFTLTISSLQ
PEDFATYYCQQRDSYLWTFGQGTKVEIKRTPREPQVYTLPPSRDELTKNQVSLKCLVKGFYPSDIAVEWESNGQPENNY
KTTPPVLDSDGSFFLYSKLTVDKSRWQQGNVFSCSVMHEALHNHYTQKSLSLSKSCGSGSGS

[symbol]

[symbol]

[symbol]

[symbol]

[symbol]

RTVAAPSVFIFPPSDEQLKSGTASVVCLLNNFYPREAKVQWKVD
NALQSGNSQESVTEQDSKDSTYSLSSTLTLSKADYEKHKVYACEVTHQGLSSPVTKSFNRGECDKTHTCPPCPAPELLG
GPSVFLFPPKPKDTLMISRTPEVTCVVVDVSHEDPEVKFNWYVDGVEVHNAKTKPREEQYNSTYRVVSVLTVLHQDWLN
GKEYKCKVSNKALPAPIEKTISKAKGQPREPQVCTLPPSREEMTKNQVSLSCAVKGFYPSDIAVEWESNGQPENNYKTT
PPVLDSDGSFFLVSKLTVDKSRWGGQNVFSCSVMHEALHNHYTQKSLSLSPGK
Domain arrangement:
R- S- linker- H- I- Hinge- J- K-
VL- CH3- linker- VL- CL- Hinge- CH2- CH3(hole)
Mutations in domain S:
T366K; 445K, 446S, 447C insertion
Six amino acids linker insertion: GSGSGS
Mutations in domain K:
Y349C, D356E, L358M, T366S, L368A, Y407V
>BC28-1x1x1a domain R [SEQ ID NO: 46]
DIQMTQSPSSLSASVGDRVTITCRASQSVSSAVAWYQQKPGKAPKLLIYSASSLYSGVPSRFSGSRSGTDFTLTISSLQ
PEDFATYYCQQRDSYLWTEGQGTKVEIKRT
>BC28-1x1x1a domain S[SEQ ID NO: 47]
PREPQVYTLPPSRDELTKNQVSLKCLVKGFYPSDIAVEWESNGQPENNYKTTPPVLDSDGSFELYSKLTVDKSRWQQGN
VESCSVMHEALHNHYTQKSLSLSKSC
>BC28-1x1x1a linker [SEQ ID NO: 48]
GSGSGS
>BC28-1x1x1a domain H [SEQ ID NO: 49]
EIVLTQSPATLSLSPGERATLSCRASQSVSSYLAWYQQKPGQAPRLLIYDASNRATGIPARFSGSGSGTDFTLTISSLE
PEDFAVYYCQQSSNWPRTFGQGTKVEIK
>BC28-1x1x1a domain I [SEQ ID NO: 50]
RTVAAPSVFIFPPSDEQLKSGTASVVCLLNNEYPREAKVQWKVDNALQSGNSQESVTEQDSKDSTYSLSSTLTLSKADY
EKHKVYACEVTHQGLSSPVTKSENRGEC
>BC28-1x1x1a domain J [SEQ ID NO: 51]
APELLGGPSVFLFPPKPKDTLMISRTPEVTCVVVDVSHEDPEVKFNWYVDGVEVHNAKTKPREEQYNSTYRVVSVLTVL
HQDWLNGKEYKCKVSNKALPAPIEKTISKAK
>BC28-1x1x1a domain K[SEQ ID NO: 52]
GQPREPQVCTLPPSREEMTKNQVSLSCAVKGFYPSDIAVEWESNGQPENNYKTTPPVLDSDGSFFLVSKLTVDKSRWQQ
GNVFSCSVMHEALHNHYTQKSLSLSPGK
>hCTLA4-4.chain 2 [SEQ ID NO: 53]
EVQLVESGGGLVQPGGSLRLSCAASGFTFSTYYIEWVRQAPGKGLEWVAVIYPYTGFTYYADSVKGRFTISADTSKNTA
YLQMNSLRAEDTAVYYCARGEYTVLDYWGQGTLVTVSSASPREPQVYTDPPSRDELTKNQVSLTCLVKGFYPSDIAVEW
ESNGQPENNYKTTPPVLDSDGSFFLYSKLTVDKSRWQQGNVFSCSVMHEALHNHYTQKSLSLSGEC
Domain arrangement:
F- G
VH- CH3
Mutations in domain G
L351D, 445G, 446E, 447C insertion
>hCTLA4-4 domain F[SEQ ID NO: 54]
EVQLVESGGGLVQPGGSLRLSCAASGFTFSTYYIEWVRQAPGKGLEWVAVIYPYTGFTYYADSVKGRFTISADTSKNTA
YLQMNSLRAEDTAVYYCARGEYTVLDYWGQGTLVTVSSAS
>hCTLA4-4 domain G [SEQ ID NO: 55]
PREPQVYTDPPSRDELTKNQVSLTCLVKGFYPSDIAVEWESNGQPENNYKTTPPVLDSDGSFFLYSKLTVDKSRWQQGN
VFSCSVMHEALHNHYTQKSLSLSGEC
Other Sequences:
>Hinge: DKTHTCPPCP [SEQ ID NO: 56]
>BC1-Polypeptide 1 Domain Junction: IKRTPREP [SEQ ID NO: 57]
>BC15-Polypeptide 1 Domain Junction: IKRTVREP [SEQ ID NO: 58]
>BC16-Polypeptide 1 Domain Junction: IKRTREP [SEQ ID NO: 59]
>BC17-Polypeptide 1 Domain Junction: IKRTVPREP [SEQ ID NO: 60]
>BC26-Polypeptide 1 Domain Junction: IKRTVAEP [SEQ ID NO: 61]
>BC27-Polypeptide 1 Domain Junction: IKRTVAPREP [SEQ ID NO: 62]
>BC1-Polypeptide 2 Domain Junction: SSASPREP [SEQ ID NO: 63]
>BC13-Polypeptide 2 Domain Junction: SSASTREP [SEQ ID NO: 64]
>BC14-Polypeptide 2 Domain Junction: SSASTPREP [SEQ ID NO: 65]
>BC24-Polypeptide 2 Domain Junction: SSASTKGEP [SEQ ID NO: 66]
>BC25-Polypeptide 2 Domain Junction: SSASTKGREP SEQ ID NO: 67]
>SP34-89 VH [SEQ ID NO: 68]
EVQLVESGGGLVQPGGSLRLSCAASGFTFNTYAMNWVRQAPGKGLEWVARIRSKYNNYATYYADSVKGRFSISRDDSKN
TAYLQMNSLRAEDTAVYYCVRHGNFGNSYVSWFAYWGQGTLVTV
>SP34-89 VL [SEQ ID NO: 69]
QAVVTQEPSLTVSPGGTVTLTCRSSTGAVTTSNYANWVQQKPGQAPRGLIGGTNKRAPWTPARFSGSLLGGKAALTITG
AQAEDEADYYCALWYSNLWVFGGGTKLTVL
>SP34-89 VH-N30S VH [SEQ ID NO: 70] lower case denotes mutation
EVQLVESGGGLVQPGGSLRLSCAASGFTFsTYAMNWVRQAPGKGLEWVARIRSKYNNYATYYADSVKGRFSISRDDSKN
TAYLQMNSLRAEDTAVYYCVRHGNFGNSYVSWFAYWGQGTLVTV
>SP34-89 VH-G65D VH [SEQ ID NO: 71] lower case denotes mutation
EVQLVESGGGLVQPGGSLRLSCAASGFTFNTYAMNWVRQAPGKGLEWVARIRSKYNNYATYYADSVKdRFSISRDDSKN
TAYLQMNSLRAEDTAVYYCVRHGNFGNSYVSWFAYWGQGTLVTV
>SP34-89 VH-S68T VH [SEQ ID NO: 72] lower case denotes mutation
EVQLVESGGGLVQPGGSLRLSCAASGFTFNTYAMNWVRQAPGKGLEWVARIRSKYNNYATYYADSVKGRFtISRDDSKN
TAYLQMNSLRAEDTAVYYCVRHGNFGNSYVSWFAYWGQGTLVTV
>SP34-89 VL-W57G VL [SEQ ID NO: 73] lower case denotes mutation
QAVVTQEPSLTVSPGGTVTLTCRSSTGAVTTSNYANWVQQKPGQAPRGLIGGTNKRAPgTPARFSGSLLGGKAALTITG
AQAEDEADYYCALWYSNLWVFGGGTKLTVL
>Phage display heavy chain [SEQ ID NO: 74]:
EVQLVESGGGLVQPGGSLRLSCAASGFTExxxx [symbol] WVRQAPGKGLEWVAxxxxxxxxxxx [symbol] RFTISADTSKNT
AYLQMNSLRAEDTAVYYCARxxxxxxxxxxxxx [symbol] WGQGTLVTVSSASTKGPSVFPLAPSSKSTSGGTAALGCLVKDYF
PEPVTVSWNSGALTSGVHTFPAVLQSSGLYSLSSVVTVPSSSLGTQTYICNVNHKPSNTKVDKKVEPKSCDKTHTCPPC
PAPELLGGPSVFLEPPKPKDTLMISRTPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSRDELTKNQVSLTCLVKGEYPSDIAVEWESNGQP
ENNYKTTPPVLDSDGSFFLYSKLTVDKSRWQQGNVFSCSVMHEALHNHYTQKSLSLSPGK
>Phage display light chain [SEQ ID NO: 75]:
DIQMTQSPSSLSASVGDRVTITC [symbol] VAWYQQKPGKAPKLLIY [symbol] [symbol] GVPSRFSGSRSGTDFTLTISSLQ
PEDFATYYC [symbol] xxxxxx [symbol] GQGTKVEIKRTVAAPSVFIFPPSDSQLKSGTASVVCLLNNFYPREAKVQWKVDNALQSGN
SQESVTEQDSKDSTYSLSSTLTLSKADYEKHKVYACEVTHQGLSSPVTKSFNRGEC
>B-Body Domain A/H Scaffold [SEQ ID NO: 76]:
DIQMTQSPSSLSASVGDRVTITC [symbol] VAWYQQKPGKAPKLLIY [symbol] [symbol] GVPSRFSGSRSGTDFTLTISSLQ
PEDFATYYC [symbol] xxxxxx [symbol] GQGTKVEIKRT
>B-Body Domain F/L Scaffold [SEQ ID NO: 77]:
EVQLVESGGGLVQPGGSLRLSCAASGFTExxxx [symbol] WVRQAPGKGLEWVAxxxxxxxxxxx [symbol] RFTISADTSKNT
AYLQMNSLRAEDTAVYYCARxxxxxxxxxxxxx [symbol] WGQGTLVTVSSAS
>BC1 Chain 1 Scaffold [SEQ ID NO: 78]
DIQMTQSPSSLSASVGDRVTITC [symbol] VAWYQQKPGKAPKLLIY [symbol] [symbol] GVPSRFSGSRSGTDFTLTISSLQ
PEDFATYYC [symbol] xxxxxx [symbol] GQGTKVEIKRTPREPQVYTLPPSRDELTKNQVSLKCLVKGFYPSDIAVEWESNGQPENNY
KTTPPVLDSDGSFFLYSKLTVDKSRWQQGNVFSCSVMHEALHNHYTQKSLSLSKSCDKTHTCPPCPAPELLGGPSVFLF
PPKPKDTLMISRTPEVTCVVVDVSHEDPEVKFNWYVDGVEVHNAKTKPREEQYNSTYRVVSVLTVLHQDWLNGKEYKCK
VSNKALPAPIEKTISKAKGQPREPQVYTLPPCRDELTKNQVSLWCLVKGFYPSDIAVEWESNGQPENNYKTTPPVLDSD
GSFFLYSKLTVDKSRWQQGNFFSCSVMHEALHNHYTQKSLSLSPGK
“x” represents CDR amino acids that were varied to create the library, and bold
italic represents the CDR sequences that were constant Domain arrangement:
A- B- Hinge- D- E
VL- CH3- Hinge- CH2- CH3(knob)
Mutations in first CH3 (Domain B):
T366K; 445K, 446S, 447C insertion
Mutations in second CH3 (Domain E):
S354C, T366W
>BC1 Chain 2 Scaffold [SEQ ID NO: 79]
EVQLVESGGGLVQPGGSLRLSCAASGFTExxxx [symbol] WVRQAPGKGLEWVAxxxxxxxxxxx [symbol] RFTISADTSKNT
AYLQMNSLRAEDTAVYYCARxxxxxxxxxxxxx [symbol] WGQGTLVTVSSASPREPQVYTDPPSRDELTKNQVSLTCLVKGFY
PSDIAVEWESNGQPENNYKTTPPVLDSDGSFFLYSKLTVDKSRWQQGNVFSCSVMHEALHNHYTQKSLSLSGEC
“x” represents CDR amino acids that were varied to create the library, and bold
italic represents the CDR sequences that were constant Domain arrangement:
F- G
VH- CH3
Mutations in CH3 (Domain G):
L351D; 445G, 446E, 447C insertion
>BC1 Chain 3 Scaffold [SEQ ID NO: 80]
DIQMTQSPSSLSASVGDRVTITC [symbol] VAWYQQKPGKAPKLLIY [symbol] [symbol] GVPSRFSGSRSGTDFTLTISSLQ
PEDFATYYC [symbol] xxxxxx [symbol] GQGTKVEIKRTVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKSFNRGECDKTHTCPPCPAPELLGGPSVFLF
PPKPKDTLMISRTPEVTCVVVDVSHEDPEVKFNWYVDGVEVHNAKTKPREEQYNSTYRVVSVLTVLHQDWLNGKEYKCK
VSNKALPAPIEKTISKAKGQPREPQVCTLPPSREEMTKNQVSLSCAVKGFYPSDIAVEWESNGQPENNYKTTPPVLDSD
GSFFLVSKLTVDKSRWQQGNVFSCSVMHEALHNHYTQKSLSLSPGK
“x” represents CDR amino acids that were varied to create the library, and bold
italic represents the CDR sequences that were constant Domain arrangement:
H- I- Hinge- J- K
VL- CL- Hinge- CH2- CH3(hole)
Mutations in CH3 (domain K):
Y349C, D356E, L358M, T366S, L368A, Y407V
>BC1 Chain 4 Scaffold [SEQ ID NO: 81]
EVQLVESGGGLVQPGGSLRLSCAASGFTFxxxx [symbol] WVRQAPGKGLEWVAxxxxxxxxxxx [symbol] RFTISADTSKNT
AYLQMNSLRAEDTAVYYCARxxxxxxxxxxxxx [symbol] WGQGTLVTVSSASTKGPSVFPLAPSSKSTSGGTAALGCLVKDYF
PEPVTVSWNSGALTSGVHTFPAVLQSSGLYSLSSVVTVPSSSLGTQTYICNVNHKPSNTKVDKKVEPPKSC
“x” represents CDR amino acids that were varied to create the library, and bold
italic represents the CDR sequences that were constant Domain arrangement:
L- M
VH- CH1
>BC1 Chain 3 1(A)x2(B-A) SP34-89 Scaffold [SEQ ID NO: 82]
DIQMTQSPSSLSASVGDRVTITC [symbol] VAWYQQKPGKAPKLLIY [symbol] [symbol] GVPSRFSGSRSGTDFTLTISSLQ
PEDFATYYC [symbol] xxxxxx [symbol] GQGTKVEIKRTPREPQVYTLPPSRDELTKNQVSLKCLVKGFYPSDIAVEWESNGQPENNY
KTTPPVLDSDGSFFLYSKLTVDKSRWQQGNVFSCSVMHEALHNHYTQKSLSLSKSCTASSGGSSSGQAVVTQEPSLTVS
PGGTVTLTCRSSTGAVTTSNYANWVQQKPGQAPRGLIGGTNKRAPWTPARFSGSLLGGKAALTITGAQAEDEADYYCAL
WYSNLWVFGGGTKLTVLGRTVAAPSVFIFPPSDEQLKSGTASVVCLLNNFYPREAKVQWKVDNALQSGNSQESVTEQDS
KDSTYSLSSTLTLSKADYEKIIKVYACEVTHQGLSSPVTKSFNRGECDKTHTCPPCPAPEAAGGPSVFLFPPKPKDTLM
ISRTPEVTCVVVDVSHEDPEVKFNWYVDGVEVHNAKTKPREEQYNSTYRVVSVLTVLHQDWLNGKEYKCKVSNKALKAP
IEKTISKAKGQPREPQVCTLPPSREEMTKNQVSLSCAVKGFYPSDIAVEWESNGQPENNYKTTPPVLDSDGSFFLVSKL
TVDKSRWQQGNVFSCSVMHEALHNHYTQKSLSLSPGK
“x” represents CDR amino acids that were varied to create the library, and bold
italic represents the CDR sequences that were constant Domain arrangement:
R- S- linker- H- I- Hinge- J- K
VL- CH3- linker- SP34-CL- Hinge- CH2- CH3(hole)
Mutations in domain S:
T366K; 445K, 446S, 447C insertion
Ten amino acids linker insertion: TASSGGSSSG
Mutations in Domain J:
L234A, L235A, and P329K
Mutations in domain K:
Y349C, D356E, L358M, T366S, L368A, Y407V
>BC1 Chain 3 1(A)x2(B-A) SP34-89 S-H Junction [SEQ ID NO: 83]
TASSGGSSSG
>Human IgA CH3 [SEQ ID NO: 84]
TFRPEVHLLPPPSEELALNELVTLTCLARGFSPKDVLVRWLQGSQELPREKYLTWASRQEPSQGTTTFAVTSILRVAAE
DWKKGDTFSCMVGHEALPLAFTQKTIDRL

### 7. INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes.

### 8. EQUIVALENTS

While various specific embodiments have been illustrated and described, the above specification is not restrictive. It will be appreciated that various changes can be made without departing from the spirit and scope of the invention(s). Many variations will become apparent to those skilled in the art upon review of this specification.

What is claimed is:

1. A trivalent trispecific binding molecule, comprising:
a first, a second, a third, a fourth, and a fifth polypeptide chain, wherein:
(a) the first polypeptide chain comprises a domain A, a domain B, a domain D, a domain E, a domain N and a domain O,
wherein the domains are arranged, from N-terminus to C-terminus, in an N-O-A-B-D-E orientation, and
domain A has a variable region domain amino acid sequence, domain B has a constant region domain amino acid sequence, domain D has a CH2 amino acid sequence, domain E has a constant region domain amino acid sequence, domain N has a variable region domain amino acid sequence, and domain O has a constant region domain amino acid sequence;
(b) the second polypeptide chain comprises a domain F and a domain G,
wherein the domains are arranged, from N-terminus to C-terminus, in a F-G orientation, and
wherein domain F has a variable region domain amino acid sequence and domain G has a constant region domain amino acid sequence amino acid sequence;
(c) the third polypeptide chain comprises a domain H, a domain I, a domain J, and a domain K,
wherein the domains are arranged, from N-terminus to C-terminus, in a H-I-J-K orientation, and
wherein domain H has a variable region domain amino acid sequence, domain I has a constant region domain amino acid sequence, domain J has a CH2 amino acid sequence, and K has a constant region domain amino acid sequence;
(d) the fourth polypeptide chain comprises a domain L and a domain M,
wherein the domains are arranged, from N-terminus to C-terminus, in a L-M orientation, and
wherein domain L has a variable region domain amino acid sequence and domain M has a constant region domain amino acid sequence;
(e) the fifth polypeptide chain comprises a domain P and a domain Q,
wherein the domains are arranged, from N-terminus to C-terminus, in a P-Q orientation, and
wherein domain P has a variable region domain amino acid sequence and domain Q has a constant region domain amino acid sequence,
(f) the first and the second polypeptides are associated through an interaction between the A and the F domains and an interaction between the B and the G domains;
(g) the third and the fourth polypeptides are associated through an interaction between the H and the L domains and an interaction between the I and the M domains;
(h) the first and the fifth polypeptides are associated through an interaction between the N and the P domains and an interaction between the O and the Q domains to form the binding molecule;
(i) the first and the third polypeptides are associated through an interaction between the D and the J domains and an interaction between the E and the K domains to form the binding molecule;
(j) the amino acid sequences of domain N, domain A, and domain H are different,
(k) the second and the fifth polypeptide chains are identical and the fourth polypeptide chain is different, or the fourth and the fifth polypeptide chains are identical and the second polypeptide chain is different, and
(l) the interaction between the A domain and the F domain form a first antigen binding site specific for a first antigen,
the interaction between the H domain and the L domain form a second antigen binding site specific for a second antigen, and
the interaction between the N domain and the P domain form a third antigen binding site specific for a third antigen.

2. The binding molecule of claim 1, wherein
the second and the fifth polypeptide chains are identical and the fourth polypeptide chain is different from the second and the fifth polypeptide chains,
the amino acid sequences of domain O and domain B are identical, and
the amino acid sequences of domain I is different from domains O and B.

3. The binding molecule of claim 1, wherein
the fourth and the fifth polypeptide chains are identical and the second polypeptide chain is different from the second and the fifth polypeptide chains,
the amino acid sequences of domain O and domain I are identical, and
the amino acid sequences of domain B is different from domains O and I.

4. A trivalent trispecific binding molecule, comprising:
a first, a second, a third, a fourth, and a sixth polypeptide chain, wherein:
(a) the first polypeptide chain comprises a domain A, a domain B, a domain D, and a domain E,
wherein the domains are arranged, from N-terminus to C-terminus, in an A-B-D-E orientation, and
domain A has a variable region domain amino acid sequence, domain B has a constant region domain amino acid sequence, domain D has a CH2 amino acid sequence, and domain E has a constant region domain amino acid sequence;
(b) the second polypeptide chain comprises a domain F and a domain G,
wherein the domains are arranged, from N-terminus to C-terminus, in a F-G orientation, and
wherein domain F has a variable region domain amino acid sequence and domain G has a constant region domain amino acid sequence amino acid sequence;
(c) the third polypeptide chain comprises a domain H, a domain I, a domain J, a domain K, a domain R, and a domain S
wherein the domains are arranged, from N-terminus to C-terminus, in a R-S-H-I-J-K orientation, and
wherein domain H has a variable region domain amino acid sequence, domain I has a constant region domain amino acid sequence, domain J has a CH2 amino acid sequence, domain K has a constant region domain amino acid sequence, domain R has a variable region domain amino acid sequence, and domain S has a constant region domain amino acid sequence;
(d) the fourth polypeptide chain comprises a domain L and a domain M,
wherein the domains are arranged, from N-terminus to C-terminus, in a L-M orientation, and
wherein domain L has a variable region domain amino acid sequence and domain M has a constant region domain amino acid sequence;
(e) the sixth polypeptide chain comprises a domain T and a domain U,
wherein the domains are arranged, from N-terminus to C-terminus, in a T-U orientation, and
wherein domain T has a variable region domain amino acid sequence and domain U has a constant region domain amino acid sequence,
(f) the first and the second polypeptides are associated through an interaction between the A and the F domains and an interaction between the B and the G domains;
(g) the third and the fourth polypeptides are associated through an interaction between the H and the L domains and an interaction between the I and the M domains;
(h) the first and the sixth polypeptides are associated through an interaction between the R and the T domains and an interaction between the S and the U domains to form the binding molecule;
(i) the first and the third polypeptides are associated through an interaction between the D and the J domains and an interaction between the E and the K domains to form the binding molecule;
(j) the amino acid sequences of domain R, domain A, and domain H are different,
(k) the second and the sixth polypeptide chains are identical and the fourth polypeptide chain is different, or the fourth and the sixth polypeptide chains are identical and the second polypeptide chain is different,
(l) the interaction between the A domain and the F domain form a first antigen binding site specific for a first antigen,
the interaction between the H domain and the L domain form a second antigen binding site specific for a second antigen, and
the interaction between the R domain R and the T domain form a third antigen binding site specific for a third antigen.

5. The binding molecule of claim 4, wherein
the fourth and the sixth polypeptide chains are identical and the fourth polypeptide chain is different from the second and the sixth polypeptide chains,
the amino acid sequences of domain S and domain I are identical, and
the amino acid sequences of domain B is different from domains S and I.

6. The binding molecule of claim 4, wherein
the second and the sixth polypeptide chains are identical and the fourth polypeptide chain is different from the second and the sixth polypeptide chains,
the amino acid sequences of domain S and domain B are identical, and
the amino acid sequences of domain I is different from domains S and B.

7. The binding molecule of any of claims 1-6, wherein the amino acid sequences of the B domain and the G domain are an endogenous CH3 sequence.

8. The binding molecule of any of claims 1-6, wherein the amino acid sequences of the B and the G domains are different and separately comprise respectively orthogonal modifications in an endogenous CH3 sequence, wherein the B domain interacts with the G domain, wherein neither the B domain nor the G domain significantly interacts with a CH3 domain lacking the orthogonal modification.

9. The binding molecule of claim 8, wherein the orthogonal modifications of the B and the G domains comprise mutations that generate engineered disulfide bridges between domain B and G.

10. The binding molecule of claim 9, wherein the mutations of the B and the G domains that generate engineered disulfide bridges are a S354C mutation in one of the B domain and G domains, and a 349C in the other domain.

11. The binding molecule of any one of claims 8-10, wherein the orthogonal modifications of the B and the G domains comprise knob-in-hole mutations.

12. The binding molecule of claim 11, wherein the knob-in hole mutations of the B and the G domains are a T366W mutation in one of the B domain and G domain, and a T366S, L368A, and aY407V mutation in the other domain.

13. The binding molecule of any one of claims 8-12, wherein the orthogonal modifications of the B and the G domains comprise charge-pair mutations.

14. The binding molecule of claim 13, wherein the charge-pair mutations of the B and the G domains are a T366K mutation in one of the B domain and G domain, and a L351D mutation in the other domain.

15. The binding molecule of any one of claims 1-14, wherein domain I has a CL sequence and domain M has a CH1 sequence.

16. The binding molecule of any one of claims 1-14, wherein domain I has a CH1 sequence and domain M has a CL sequence.

17. The binding molecule of claim 15 or 16, wherein the CH1 sequence and the CL sequence each comprise one or more orthogonal modifications, wherein a domain having the CH1 sequence does not significantly interact with a domain having a CL sequence lacking the orthogonal modification.

18. The binding molecule of claim 17, wherein the orthogonal modifications comprise mutations that generate engineered disulfide bridges between the at least one CH1 domain and a CL domain, the mutations selected from the group consisting of: an engineered cysteine at position 138 of the CH1 sequence and position 116 of the CL sequence; an engineered cysteine at position 128 of the CH1 sequence and position 119 of the CL sequence, and an engineered cysteine at position 129 of the CH1 sequence and position 210 of the CL sequence.

19. The binding molecule of claim 17, wherein the orthogonal modifications comprise mutations that generate engineered disulfide bridges between the at least one CH1 domain and a CL domain, wherein the mutations comprise and engineered cysteines at position 128 of the CH1 sequence and position 118 of a CL Kappa sequence.

20. The binding molecule of claim 17, wherein the orthogonal modifications comprise mutations that generate engineered disulfide bridges between the at least one CH1 domain and a CL domain, the mutations selected from the group consisting of: a F118C mutation in the CL sequence with a corresponding A141C in the CH1 sequence; a F118C mutation in the CL sequence with a corresponding L128C in the CH1 sequence; and a S162C mutations in the CL sequence with a corresponding P171C mutation in the CH1 sequence.

21. The binding molecule of any of claims 17-20, wherein the orthogonal modifications comprise charge-pair mutations between the at least one CH1 domain and a CL domain, the charge-pair mutations selected from the group consisting of: a F118S mutation in the CL sequence with a corresponding A141L in the CH1 sequence; a F118A mutation in the CL sequence with a corresponding A141L in the CH1 sequence; a F118V mutation in the CL sequence with a corresponding A141L in the CH1 sequence; and a T129R mutation in the CL sequence with a corresponding K147D in the CH1 sequence.

22. The binding molecule of any of claims 17-20, wherein the orthogonal modifications comprise charge-pair mutations between the at least one CH1 domain and a CL domain, the charge-pair mutations selected from the group consisting of: a N138K mutation in the CL sequence with a corresponding G166D in the CH1 sequence, and a N138D mutation in the CL sequence with a corresponding G166K in the CH1 sequence.

23. The binding molecule of any one of claims 1-22, wherein the E domain has a CH3 amino acid sequence.

24. The binding molecule of any one of claims 1-23, wherein the amino acid sequences of the E domain and the K domain are identical, wherein the sequence is an endogenous CH3 sequence.

25. The binding molecule of any one of claims 1-23, wherein the amino acid sequences of the E domain and the K domain are different.

26. The binding molecule of claim 25, wherein the different sequences separately comprise respectively orthogonal modifications in an endogenous CH3 sequence, wherein the E domain interacts with the K domain, and wherein neither the E domain nor the K domain significantly interacts with a CH3 domain lacking the orthogonal modification.

27. The binding molecule of claim 26, wherein the orthogonal modifications comprise mutations that generate engineered disulfide bridges between the E domain and the K domain.

28. The binding molecule of claim 27, wherein the mutations that generate engineered disulfide bridges are a S354C mutation in one of the E domain and the K domain, and a 349C in the other domain.

29. The binding molecule of any one of claims 26-28, wherein the orthogonal modifications in the E domain and the K domain comprise knob-in-hole mutations.

30. The binding molecule of claim 29, wherein the knob-in hole mutations are a T366W mutation in one of the E domain or the K domain and a T366S, L368A, and aY407V mutation in the other domain.

31. The binding molecule of any one of claims 26-30, wherein the orthogonal modifications in the E domain and the K domain comprise charge-pair mutations.

32. The binding molecule of claim 31, wherein the charge-pair mutations are a T366K mutation in one of the E domain or the K domain and a corresponding L351D mutation in the other domain.

33. The binding molecule of claim 25, wherein the amino acid sequences of the E domain and the K domain are endogenous sequences of two different antibody domains, the domains selected to have a specific interaction that promotes the specific association between the first and the third polypeptides.

34. The binding molecule of claim 33, wherein the two different amino acid sequences are a CH1 sequence and a CL sequence.

35. The binding molecule of any of claims 1-34, wherein domain A has a VL amino acid sequence and domain F has a VH amino acid sequence.

36. The binding molecule of any of claims 1-34, wherein domain A has a VH amino acid sequence and domain F has a VL amino acid sequence.

37. The binding molecule of any of claims 1-36, wherein domain H has a VL amino acid sequence and domain L has a VH amino acid sequence.

38. The binding molecule of any of claims 1-36, wherein domain H has a VH amino acid sequence and domain L has a VL amino acid sequence.

39. The binding molecule of any of the above claims, wherein the sequence that forms the junction between the A domain and the B domain is IKRTPREP or IKRTVREP.

40. The binding molecule of any of the above claims, wherein the sequence that forms the junction between the F domain and the G domain is SSASPREP.

41. The binding molecule of any of the above claims, wherein at least one CH3 amino acid sequence has a C-terminal tripeptide insertion connecting the CH3 amino acid sequence to a hinge amino acid sequence, wherein the tripeptide insertion is selected from the group consisting of PGK, KSC, and GEC.

42. The binding molecule of any of the above claims, wherein the sequences are human sequences.

43. The binding molecule of any of the above claims, wherein at least one CH3 amino acid sequence is an IgG sequence.

44. The binding molecule of claim 43, wherein the IgG sequences are IgG1 sequences.

45. The binding molecule of any of the above claims, wherein at least one CH3 amino acid sequence has one or more isoallotype mutations.

46. The binding molecule of claim 45, wherein the isoallotype mutations are D356E and L358M.

47. The binding molecule of any of the above claims, wherein the CL amino acid sequence is a Ckappa sequence.

48. The binding molecule of any of the above claims, wherein the CH2 sequences have one or more engineered mutations that reduce Fc effector function.

49. The binding molecule of claim 48, wherein the one or more engineered mutations are at position L234, L235, and P329.

50. The binding molecule of claim 49, wherein the one or more engineered mutations are L234A, L235A, and P329G.

51. The binding molecule of claim 49, wherein the one or more engineered mutations are L234A, L235A, and P329K.

52. A purified binding molecule, the purified binding molecule comprising the binding molecule of any one of claims 1-51 purified by a purification method comprising a CH1 affinity purification step.

53. The purified binding molecule of claim 52, wherein the purification method is a single-step purification method.

54. A pharmaceutical composition comprising the binding molecule of any one of claims 1-53 and a pharmaceutically acceptable diluent.

55. A method for treating a subject with cancer, the method comprising administering a therapeutically effective amount of the pharmaceutical composition of claim 54 to the subject.
